(19) United States
(12) Patent Application Publication
(10) Pub. No.: US 2013/0165343 A1
(43) Pub. Date: Jun. 27, 2013

(54) IDENTIFICATION OF MULTIGENE BIOMARKERS

(71) Applicant: AVEO Pharmaceuticals, Inc., Cambridge, MA (US)

(72) Inventors: Murray Robinson, Boston, MA (US); Bin Feng, Newton, MA (US); Richard Nicoletti, Southborough, MA (US); Joshua P. Frederick, Boston, MA (US); Lejla Pilipovic, Somerville, MA (US)

(73) Assignee: AVEO Pharmaceuticals, Inc., Cambridge, MA (US)

(21) Appl. No.: 13/775,928

(22) Filed: Feb. 25, 2013

Related U.S. Application Data
(62) Division of application No. 13/669,275, filed on Nov. 5, 2012.
(60) Provisional application No. 61/579,530, filed on Dec. 22, 2011.

(51) Int. Cl. C12Q 1/68

(52) U.S. Cl. CPC C12Q 1/6837; C12Q 1/686; G06F 19/34
USPC 506/9; 435/6.12; 702/19

(57) ABSTRACT

Methods for identifying multigene biomarkers for predicting sensitivity or resistance to an anti-cancer drug of interest, or multigene cancer prognostic biomarkers are disclosed. The disclosed methods are based on the classification of the mammalian genome into 51 transcription clusters, i.e., non-overlapping, functionally relevant groups of genes whose intra-group transcript levels are highly correlated. Also disclosed are specific multigene biomarkers for predicting sensitivity or resistance to tivozanib, or rapamycin, and a specific multigene biomarker for determining breast cancer prognosis, all of which were identified using the methods disclosed herein.

### CROSS-REFERENCE TO RELATED APPLICATIONS

This application is a divisional of U.S. patent application Ser. No. 13/669,275, filed Nov. 5, 2012, which claims the benefit of and priority to U.S. provisional application Ser. No. 61/579,530, filed Dec. 22, 2011; the entire contents of each of which are incorporated herein by reference.

### FIELD OF THE INVENTION

The field of the invention is molecular biology, genetics, oncology, bioinformatics and diagnostic testing.

### BACKGROUND

Most cancer drugs are effective in some patients, but not others. This results from genetic variation among tumors, and can be observed even among tumors within the same patient. Variable patient response is particularly pronounced with respect to targeted therapeutics. Therefore, the full potential of targeted therapies cannot be realized without suitable tests for determining which patients will benefit from which drugs. According to the National Institutes of Health (NIH), the term “biomarker” is defined as “a characteristic that is objectively measured and evaluated as an indicator of normal biologic or pathogenic processes or pharmacological response to a therapeutic intervention.”

The development of improved diagnostics based on the discovery of biomarkers has the potential to accelerate new drug development by identifying, in advance, those patients most likely to show a clinical response to a given drug. This would significantly reduce the size, length and cost of clinical trials. Technologies such as genomics, proteomics and molecular imaging currently enable rapid, sensitive and reliable detection of specific gene mutations, expression levels of particular genes, and other molecular biomarkers. In spite of the availability of various technologies for molecular characterization of tumors, the clinical utilization of cancer biomarkers remains largely unrealized because few cancer biomarkers have been discovered. For example, a recent review article states:
There is a critical need for expedited development of biomarkers and their use to improve diagnosis and treatment of cancer. (Cho, 2007, Molecular Cancer 6:25)

Another recent review article on cancer biomarkers contains the following comments:
The challenge is discovering cancer biomarkers. Although there have been clinical successes in targeting molecularly defined subsets of several tumor types—such as chronic myeloid leukemia, gastrointestinal stromal tumor, lung cancer and glioblastoma multiforme—using molecularly targeted agents, the ability to apply such successes in a broader context is severely limited by the lack of an efficient strategy to evaluate targeted agents in patients. The problem mainly lies in the inability to select patients with molecularly defined cancers for clinical trials to evaluate these exciting new drugs. The solution requires biomarkers that reliably identify those patients who are most likely to benefit from a particular agent. (Sawyers, 2008, Nature 452:548-552, at 548)
   Comments such as the foregoing illustrate the recognition of a need for the discovery of clinically useful predictive biomarkers, particularly in the field of oncology.

There is a well-recognized need for methods of identifying multigene biomarkers for identifying which patients are suitable candidates for treatment with a given drug or therapy. This is particularly true with regard to targeted cancer therapeutics.

### SUMMARY

Using gene expression profiling technologies, proprietary bioinformatics tools, and applied statistics, we have discovered that the mammalian genome can be usefully represented by 51 non-overlapping, functionally relevant groups of genes whose intra-group transcript level is coordinately regulated, i.e., strongly correlated, or “coherent,” across various microarray datasets. We have designated these groups of genes Transcription Clusters 1-51 (TC1-TC51). Based on this discovery, we have discovered a broadly applicable method for rapidly identifying: (a) a multigene predictive biomarker for sensitivity or resistance to an anti-cancer drug of interest; or (b) a multigene cancer prognostic biomarker. We call such a multigene biomarker a Predictive Gene Set, or PGS.

A PGS can be based on one transcription cluster or a multiplicity of transcription clusters. In some embodiments, a PGS is based on one or more transcription clusters in their entirety. In other embodiments, the PGS is based on a subset of genes in a single transcription cluster or subsets of a multiplicity of transcription clusters. A subset of genes from any given transcription cluster is representative of the entire transcription cluster from which it is taken, because expression of the genes within that transcription cluster is coherent. Thus, when a subset of genes in a transcription cluster is used, the subset is a representative subset of genes from the transcription cluster.

Provided herein is a method for identifying a predictive gene set (“PGS”) for classifying a cancerous tissue as sensitive or resistant to a particular anticancer drug or class of drug. The method comprises the steps of (a) measuring expression levels of a representative number of genes (such as 10, 15, 20 or more genes) from a transcription cluster in Table 1, in (i) a set of tissue samples from a population of cancerous tissues identified as sensitive to the anticancer drug, and (ii) a set of a tissue samples from a population of cancerous tissues identified as resistant to the anticancer drug; and (b) determining whether there is a statistically significant difference between the expression levels of the representative number of genes in the set of tissue samples from the sensitive population, and the set of tissue samples from the resistant population. A representative number of genes whose gene expression levels in the sensitive population are significantly different from its gene expression levels in the resistant population is a PGS for classifying a sample as sensitive or resistant to the anticancer drug. A Student's t test or Gene Set Enrichment Analysis (GSEA) can be used for determining whether there is a statistically significant difference between the expression levels of the representative number of genes in the set of tissue samples from the sensitive population and the set of tissue samples from the resistant population. In some embodiments, steps (a) and (b) are performed for each of the 51 transcription clusters disclosed herein. The tissue sample may be a tumor sample or a blood sample.

Provided herein is another method for identifying a PGS for classifying a cancerous tissue as sensitive or resistant to a particular anticancer drug or class of drug. The method comprises (a) measuring the expression levels of the ten genes in FIG. 6 representing each of the 51 transcription clusters in: (i) a set of tissue samples from a population of cancerous tissues identified as sensitive to the anticancer drug, and (ii) a set of tissue samples from a population of cancerous tissues identified as resistant to the anticancer drug; and (b) determining for each of the 51 transcription clusters whether there is a statistically significant difference between the expression levels of the ten genes in FIG. 6 that represent that cluster in the set of tissue samples from the sensitive population, and the set of tissue samples from the resistant population. In some embodiments, a transcription cluster, as represented by the ten genes from that cluster in FIG. 6 and exhibiting gene expression levels in the sensitive population which are significantly different from gene expression levels in the resistant population, is a PGS for classifying a sample as sensitive or resistant to the anticancer drug. In other embodiments, the PGS is based on a multiplicity of transcription clusters. The tissue sample may be a tumor sample or a blood sample.

Provided herein is a method for identifying a PGS for classifying a cancer patient as having a good prognosis or a poor prognosis. The method comprises (a) measuring the expression levels of a representative number of genes (such as 10, 15, 20 or more genes) from a transcription cluster in Table 1 in: (i) a set of tissue samples from a population of cancer patients identified as having a good prognosis, and (ii) a set of tissue samples from a population of cancer patients identified as having a poor prognosis; and (b) determining whether there is a statistically significant difference between the expression levels of the representative number of genes in the set of tissue samples from the good prognosis population, and the set of tissue samples from the poor prognosis population. A representative number of genes whose gene expression levels in the good prognosis population are significantly different from its gene expression levels in the poor prognosis population is a PGS for classifying a patient as having a good prognosis or poor prognosis. A Student's t test or Gene Set Enrichment Analysis (GSEA) can be used for determining whether there is a statistically significant difference between the expression levels of the representative number of genes in the set of tissue samples from the good prognosis population and the set of tissue samples from the poor prognosis population. In some embodiments, steps (a) and (b) are performed for each of the 51 transcription clusters disclosed herein. The tissue sample may be a tumor sample or a blood sample.

Provided herein is another method for identifying a PGS for classifying a cancer patient as having a good prognosis or a poor prognosis. The method comprises (a) measuring the expression levels of the ten genes in FIG. 6 representing each of the 51 transcription clusters in: (i) a set of tissue samples from a population of cancer patients identified as having a good prognosis, and (ii) a set of tissue samples from a population of cancer patients identified as having a poor prognosis; and (b) determining for each of the 51 transcription clusters whether there is a statistically significant difference between the expression levels of the ten genes in FIG. 6 that represent that cluster in the set of tissue samples from the good prognosis population, and the set of tissue samples from the poor prognosis population. In some embodiments, a transcription cluster, as represented by the ten genes from that cluster in FIG. 6, whose gene expression levels in the good prognosis population are significantly different from its gene expression levels in the poor prognosis population is a PGS for classifying a patient as having a good prognosis or poor prognosis. In other embodiments, the PGS is based on a multiplicity of transcription clusters. The tissue sample may be a tumor sample or a blood sample.

Provided herein is a method of identifying a human tumor as likely to be sensitive or resistant to treatment with the anti-cancer drug tivozanib. The method comprises (a) measuring, in a sample from the tumor, the relative expression level of each gene in a PGS that comprises at least 10 of the genes from TC50; and (b) calculating a PGS score according to the algorithm

$$P G S . score = \frac{1}{n} * {\sum }_{i = 1}^{n} Ei$$

wherein E1, E2, . . . En are the expression values of the n of genes in the PGS, wherein n is the number of genes in the PGS, and wherein a PGS score below a defined threshold indicates that the tumor is likely to be sensitive to tivozanib, and a PGS score above the defined threshold indicates that the tumor is likely to be resistant to tivozanib. In one embodiment, the PGS comprises a 10-gene subset of TC50. An exemplary 10-gene subset from TC50 is MRC1, ALOX5AP, TM6SF1, CTSB, FCGR2B, TBXAS1, MS4A4A, MSR1, NCKAP1L, and FLI1. Another exemplary 10-gene subset from TC50 is LAPTM5, FCER1G, CD48, BIN2, C1QB, NCF2, CD14, TLR2, CCL5, and CD163.

In some embodiments, the method of identifying a human tumor as likely to be sensitive or resistant to treatment with tivozanib includes performing a threshold determination analysis, thereby generating a defined threshold. The threshold determination analysis can include a receiver operator characteristic curve analysis. The relative gene expression level for each gene in the PGS can be determined (e.g., measured) by DNA microarray analysis, qRT-PCR analysis, qNPA analysis, a molecular barcode-based assay, or a multiplex bead-based assay.

Provided herein is a method of identifying a human tumor as likely to be sensitive or resistant to treatment with rapamycin. The method comprises (a) measuring, in a sample from the tumor, the relative expression level of each gene in a PGS that comprises (i) at least 10 genes from TC33; and (ii) at least 10 genes from TC26; and (b) calculating a PGS score according to the algorithm:

$$P G S . score = ( \frac{1}{m ,} * {\sum }_{i = 1}^{m} Ei - \frac{1}{n} * {\sum }_{j = 1}^{n} Fj ) / 2$$

wherein E1, E2, . . . Em are the expression values of the m genes from TC33 (for example, wherein m is at least 10 genes), which are up-regulated in sensitive tumors; and F1, F2, Fn are the expression values of n genes from TC26 (for example, wherein n is at least 10 genes), which are up-regulated in resistant tumors. A PGS score above the defined threshold indicates that the tumor is likely to be sensitive to rapamycin, and a PGS score below the defined threshold indicates that the tumor is likely to be resistant to rapamycin. An exemplary PGS comprises the following genes: FRY, HLF, HMBS, RCAN2, HMGA1, ITPR1, ENPP2, SLC16A4, ANK2, PIK3R1, DTL, CTPS, GINS2, GMNN, MCM5, PRIM1, SNRPA, TK1, UCK2, and PCNA.

In some embodiments, the method of identifying a human tumor as likely to be sensitive or resistant to treatment with rapamycin includes performing a threshold determination analysis, thereby generating a defined threshold. The threshold determination analysis can include a receiver operator characteristic curve analysis. The relative gene expression level for each gene in the PGS can be determined (e.g., measured) by DNA microarray analysis, qRT-PCR analysis, qNPA analysis, a molecular barcode-based assay, or a multiplex bead-based assay.

Provided herein is a method of classifying a human breast cancer patient as having a good prognosis or a poor prognosis. The method comprises (a) measuring, in a sample from a tumor obtained from the patient, the relative expression level of each gene in a PGS that comprises (i) at least 10 genes from TC35; and (ii) at least 10 genes from TC26; and (b) calculating a PGS score according to the algorithm:

$$P G S . score = ( \frac{1}{m ,} * {\sum }_{i = 1}^{m} Ei - \frac{1}{n} * {\sum }_{j = 1}^{n} Fj ) / 2$$

wherein E1, E2, . . . Em are the expression values of the m genes from TC35 (for example, wherein m is at least 10 genes), which are up-regulated in good prognosis patients; and F1, F2, . . . Fn are the expression values of the n genes from TC26 (for example, wherein n is at least 10 genes), which are up-regulated in poor prognosis patients. A PGS score above the defined threshold indicates that the patient has a good prognosis, and a PGS score below the defined threshold indicates that the patient is likely to have a poor prognosis. An exemplary PGS comprises the following genes: RPL29, RPL36A, RPS8, RPS9, EEF1B2, RPS10P5, RPL13A, RPL36, RPL18, RPL14, DTL, CTPS, GINS2, GMNN, MCM5, PRIM1, SNRPA, TK1, UCK2, and PCNA.

In some embodiments, the method of classifying a human breast cancer patient as having a good prognosis or a poor prognosis include performing a threshold determination analysis, thereby generating a defined threshold. The threshold determination analysis can include a receiver operator characteristic curve analysis. The relative gene expression level for each gene in the PGS can be determined (e.g., measured) by DNA microarray analysis, qRT-PCR analysis, qNPA analysis, a molecular barcode-based assay, or a multiplex bead-based assay.

Provided herein is a probe set comprising probes for at least 10 genes from each transcription cluster in Table 1, provided that the probe set is not a whole-genome microarray chip. Examples of suitable probe sets include a microarray probe set, a set of PCR primers, a qNPA probe set, a probe set comprising molecular bar codes (e.g., NanoString® Technology) or a probe set wherein probes are affixed to beads (e.g., QuantiGene® Plex assay system). In one embodiment, the probe set comprises probes for each of the 510 genes listed in FIG. 6. In another embodiment, the probe set consists of probes for each of the 510 genes listed in FIG. 6, and a control probe. In another embodiment, the probe set comprises probes for 10 genes from each transcription cluster in Table 1, wherein the probe set comprises probes for at least five genes from each transcription cluster as shown in FIG. 6, and up to five genes from each corresponding transcription cluster randomly selected from each transcription cluster in Table 1, and, optionally, a control probe. In certain embodiments, a probe set comprises between about 510-1,020 probes, 510-1,530 probes, 510-2,040 probes, 510-2,550 probes, or 510-5,100 probes.

These and other aspects and advantages of the invention will become apparent upon consideration of the following figures, detailed description, and claims.

### BRIEF DESCRIPTION OF THE DRAWINGS

FIG. 1 is a waterfall plot that summarizes data from Example 3, which is an experiment demonstrating the predictive power of the tivozanib PGS identified in Example 2. Each bar represents one tumor in the population of 25 tumors. The tumors are arranged by PGS Score (low to high). The PGS Score of each tumor is represented by the height of the bar. Actual responders (tivozanib sensitive) are indicated by black bars; actual non-responders (tivozanib resistant) are identified by gray bars. Predicted responders are those below the PGS Score optimum threshold value, which was calculated to be 1.62 (represented by the horizontal dotted line). Predicted non-responders are those above the threshold value.

FIG. 2 is a receiver operator characteristic (ROC) curve based on the data in FIG. 1. In general, a ROC curve is used to determine the optimum threshold. The ROC curve in FIG. 2 indicated that the optimum threshold PGS Score in this experiment is 1.62. When this threshold is applied, the test correctly classified 22 out of the 25 tumors, with a false positive rate of 25% and a false negative rate of 0%.

FIG. 3 is a waterfall plot that summarizes data from Example 5, which is an experiment demonstrating the predictive power of the rapamycin PGS identified in Example 4. Each bar represents one tumor in the population of 66 tumors. The tumors are arranged by PGS Score (low to high). The PGS Score of each tumor is represented by the height of the bar. Actual responders are indicated by black bars; actual non-responders are identified by gray bars. Predicted responders are those below the PGS Score optimum threshold value, which was calculated to be 0.011 (represented by the horizontal dotted line). Predicted non-responders are those above the threshold value.

FIG. 4 is a receiver operator characteristic (ROC) curve based on the data in FIG. 3. The ROC curve in FIG. 4 indicated that the optimum threshold PGS Score in this experiment is −0.011. When this threshold is applied, the test correctly classified 45 out of the 66 tumors, with a false positive rate of 16% and a false negative rate of 41%.

FIG. 5 is a comparison of Kaplan-Meier survivor curves generated by using the PGS in Example 6 to classify a population of 286 breast cancer patients represented in the Wang breast cancer dataset, as described in Example 7. This plot shows the percentage of patients surviving versus time (in months). The upper curve represents patients with high PGS scores (scores above the threshold), which patients achieved relatively longer actual survival. The lower curve, represents patients with low PGS scores (scores below the threshold), which patients achieved relatively shorter actual survival. Cox proportional hazards regression model analysis showed that the PGS generated from TC35 and TC26 is an effective prognostic biomarker, with a p-value of 4.5e-4, and a hazard ratio of 0.505. Hashmarks denote censored patients.

FIG. 6 is a table that lists 510 human genes, wherein each of the 51 transcription clusters in Table 1 is represented by a subset of 10 genes.

### DETAILED DESCRIPTION

### Definitions

As used herein, “coherence” means, when applied to a set of genes, that expression levels of the members of the set display a statistically significant tendency to increase or decrease in concert, within a given type of tissue, e.g., tumor tissue. Without intending to be bound by theory, the inventors note that coherence is likely to indicate that the coherent genes share a common involvement in one or more biological functions.

As used herein, “optimum threshold PGS score” means the threshold PGS score at which the classifier gives the most desirable balance between the cost of false negative calls and false positive calls.

As used herein, “Predictive Gene Set” or “PGS” means, with respect to a given phenotype, e.g., sensitivity or resistance to a particular cancer drug, a set of ten or more genes whose PGS score in a given type of tissue sample significantly correlates with the given phenotype in the given type of tissue.

As used herein, “good prognosis” means that a patient is expected to have no distant metastases of a tumor within five years of initial diagnosis of cancer.

As used herein, “poor prognosis” means that a patient is expected to have distant metastases of a tumor within five years of initial diagnosis of cancer.

As used herein, “probe” means a molecule that can be used for measuring the expression of a particular gene. Exemplary probes include PCR primers, as well as gene-specific DNA oligonucleotide probes such as microarray probes affixed to a microarray substrate, quantitative nuclease protection assay probes, probes linked to molecular barcodes, and probes affixed to beads.

As used herein, “receiver operating characteristic” (ROC) curve means a graphical plot of false positive rate (sensitivity) versus true positive rate (specificity) for a binary classifier system. In construction of an ROC curve, the following definitions apply:

False negative rate: FNR=1−TPR

True positive rate: TPR=true positive/(true positive+false negative)

False positive rate: FPR=false positive/(false positive+true negative)

As used herein, “response” or “responding” to treatment means, with regard to a treated tumor, that the tumor displays: (a) slowing of growth, (b) cessation of growth, or (c) regression. A tumor that responds to therapy is a “responder” and is “sensitive” to treatment. A tumor that does not respond to therapy is a “non-responder” and is “resistant” to treatment.

As used herein, “threshold determination analysis” means analysis of a dataset representing a given tumor type, e.g., human renal cell carcinoma, to determine a threshold PGS score, e.g., an optimum threshold PGS score, for that particular tumor type. In the context of a threshold determination analysis, the dataset representing a given tumor type includes (a) actual response data (response or non-response), and (b) a PGS score for each tumor from a group of tumor-bearing mice or humans.

### Transcription Clusters

Current thinking among many biologists is that the approximately 25,000 genes expressed in mammals are subject to complex regulation in order to carry out the development and function of the organism. Groups of genes function together in coordinated systems such as DNA replication, protein synthesis, neural development, etc. Currently, there is no comprehensive methodology for studying and characterizing coordinated expression of genes across the entire genome, at the transcriptional level.

We set out to group, or “bin,” genes into different functional groups or pathways, based on expression microarray data. We developed a stepwise statistical methodology to identify sets of coordinately regulated genes. The first step was to calculate a correlation coefficient for the expression level of every gene with respect to every other gene, in each of eight human datasets. This resulted in a 13,000 by 13,000 matrix of correlation scores based on data from commercial microarray chips (Affymetrix U133A). K-means clustering then was carried out across the 13,000 by 13,000 matrix of correlation scores. Because the 13,000 genes on the microarray chips are scattered across the entire human genome, and because these 13,000 genes are generally considered to include the most important human genes, the 13,000-gene chips are considered “whole genome” microarrays.

Historically, many investigators have found correlations between expression levels of certain genes and a biological condition or phenotype of interest. Such correlations, however, have had very limited usefulness. This is because the correlations typically do not hold up across datasets, e.g., human breast tumors vs. mouse breast tumors; human breast tumors vs. human lung tumors; or one gene expression technology platform (Affymetrix) vs. another gene expression technology platform (Agilent).

We have avoided this pitfall by identifying gene expression correlations that are observed across multiple, diverse datasets. By applying K-means cluster analysis (Lloyd et al., 1982, IEEE Transactions on Information Theory 28:129-137) to measured RNA expression values for all 13,000 human genes, across multiple independent data sets, we sorted the universe of transcribed human genes, the “transcriptome,” into 100 unique, non-overlapping sets of genes whose expression levels, in terms of transcriptional flux, move (increase or decrease) together. The coordinated variation in gene transcript level observed across multiple data sets is an empirical phenomenon that we call “coherence.”

After identifying the 100 non-overlapping gene groups through K-means cluster analysis, we performed an optimization process that included the following steps: (a) application of a coherency threshold, which eliminated outliers (individual genes) within each of the 100 groups; (b) identification and removal of individual genes whose expression value varied excessively, when tested in an Affymetrix system versus an Agilent system; and (c) application of threshold for minimum number of genes in any cluster, after steps (a) and (b). The end result of this optimization process was a set of 51 defined, highly coherent, non-overlapping, gene lists which we call “transcription clusters.” By mathematically reducing the complexity of a biological system containing tens of thousands of genes down to 51 groups of genes that can be represented by as few as ten genes per group, this set of 51 transcription clusters has proven to be a powerful tool for interpreting and utilizing gene expression data. The genes in each transcription cluster are listed in Table 1 (below) and identified by both Human Genome Organization (HUGO) symbol and Entrez Identifier.

TABLE 1
Transcription Clusters

| | |
|---|---|
| HUGO | Entrez |
| symbol | Identifier |

TC 1

| | |
|---|---|
| APOBEC3A | 200315 |
| CYB5R2 | 51700 |
| DSC3 | 1825 |
| DSG3 | 1830 |
| GPR87 | 53836 |
| KRT13 | 3860 |
| KRT14 | 3861 |
| KRT15 | 3866 |
| KRT5 | 3852 |
| KRT6A | 3853 |
| LY6D | 8581 |
| MMP10 | 4319 |
| NIACR2 | 8843 |
| NTS | 4922 |
| S100A7 | 6278 |
| SERPINB4 | 6318 |
| SPRR1A | 6698 |
| SPRR1B | 6699 |
| SPRR3 | 6707 |
| ZNF750 | 79755 |

TC 2

| | |
|---|---|
| AFM | 173 |
| AKR1C4 | 1109 |
| ALDH1L1 | 10840 |
| ALDH7A1 | 501 |
| APOA2 | 336 |
| APOB | 338 |
| APOH | 350 |
| C8G | 733 |
| CLDN15 | 24146 |
| CPB2 | 1361 |
| CYP2B6 | 1555 |
| CYP3A7 | 1551 |
| FBXO7 | 25793 |
| FGA | 2243 |
| GC | 2638 |
| GLUD2 | 2747 |
| GPR88 | 54112 |
| HABP2 | 3026 |
| HAL | 3034 |
| MBNL3 | 55796 |
| MTTP | 4547 |
| NR1H4 | 9971 |
| NR5A2 | 2494 |
| PECR | 55825 |
| PEPD | 5184 |
| PON3 | 5446 |
| PRG4 | 10216 |
| RELN | 5649 |
| SEPW1 | 6415 |
| SLC2A2 | 6514 |
| SLC6A1 | 6529 |
| TF | 7018 |
| UGT2B15 | 7366 |

TC 3

| | |
|---|---|
| ACOT11 | 26027 |
| AIM1L | 55057 |
| APOBEC1 | 339 |
| C17ORF73 | 55018 |
| CAPN9 | 10753 |
| CEACAM7 | 1087 |
| CFTR | 1080 |
| CLCA1 | 1179 |
| CST2 | 1470 |
| CYP2C18 | 1562 |
| DEFA6 | 1671 |
| DMBT1 | 1755 |
| EPHB2 | 2048 |
| EPS8L3 | 79574 |
| FAM127B | 26071 |
| FOXA2 | 3170 |
| FUT6 | 2528 |
| GUCY2C | 2984 |
| IHH | 3549 |
| ITPKA | 3706 |
| KLK10 | 5655 |
| MUC2 | 4583 |
| MUPCDH | 53841 |
| MYO1A | 4640 |
| PCDH24 | 54825 |
| PLEKHG6 | 55200 |
| PPP1R14D | 54866 |
| PRSS1 | 5644 |
| PRSS2 | 5645 |
| PTPRH | 5794 |
| REG3A | 5068 |
| RNF186 | 54546 |
| RNF43 | 54894 |
| SGK2 | 10110 |
| SLC26A3 | 1811 |
| SLC35D1 | 23169 |
| SLC6A20 | 54716 |
| SPINK4 | 27290 |
| SULT1B1 | 27284 |
| TFF2 | 7032 |
| TM4SF20 | 79853 |
| TM4SF5 | 9032 |
| TRIM31 | 11074 |

TC 4

| | |
|---|---|
| ABHD11 | 83451 |
| ABP1 | 26 |
| AKAP1 | 8165 |
| ARHGEF5 | 7984 |
| ARL14 | 80117 |
| ARL4A | 10124 |
| ASS1 | 445 |
| ATP10B | 23120 |
| BAK1 | 578 |
| BNIP3 | 664 |
| BSPRY | 54836 |
| C16ORF5 | 29965 |
| C1ORF116 | 79098 |
| C6ORF105 | 84830 |
| CALML4 | 91860 |
| CAP2 | 10486 |
| CAPN1 | 823 |
| CCND2 | 894 |
| CDH1 | 999 |
| CEACAM1 | 634 |
| CEACAM5 | 1048 |
| CLDN3 | 1365 |
| CNKSR1 | 10256 |
| CORO2A | 7464 |
| CTSE | 1510 |
| CXADR | 1525 |
| DDC | 1644 |
| DNMBP | 23268 |
| DTX4 | 23220 |
| EHF | 26298 |
| ELL3 | 80237 |
| ENTPD6 | 955 |
| EPB41L4B | 54566 |
| EVI1 | 2122 |
| FAR2 | 55711 |
| FUT4 | 2526 |
| FXYD3 | 5349 |
| GIPC2 | 54810 |
| GNB5 | 10681 |
| GPR35 | 2859 |
| HNF4G | 3174 |
| HSD11B2 | 3291 |
| IL1R2 | 7850 |
| LDOC1 | 23641 |
| LLGL2 | 3993 |
| LPCAT4 | 254531 |
| MAP7 | 9053 |
| MICALL2 | 79778 |
| MMP12 | 4321 |
| MST1R | 4486 |
| OAZ2 | 4947 |
| OBSL1 | 23363 |
| OLFM4 | 10562 |
| PDZK1 | 5174 |
| PIP5K1B | 8395 |
| PKP2 | 5318 |
| PLA2G10 | 8399 |
| PLP2 | 5355 |
| PTK6 | 5753 |
| RAPGEFL1 | 51195 |
| RICS | 9743 |
| RNF128 | 79589 |
| SELENBP1 | 8991 |
| SH2D3A | 10045 |
| SLC37A1 | 54020 |
| SLC39A4 | 55630 |
| SLCO4A1 | 28231 |
| SLPI | 6590 |
| SPINK1 | 6690 |
| SPINT1 | 6692 |
| STAP2 | 55620 |
| STYK1 | 55359 |
| SULT1A3 | 6818 |
| TFCP2L1 | 29842 |
| TIMM22 | 29928 |
| TMEM62 | 80021 |
| TNFRSF11A | 8792 |
| TRIM2 | 23321 |
| TSPAN15 | 23555 |
| USH1C | 10083 |
| VIL1 | 7429 |
| VILL | 50853 |
| WDR91 | 29062 |
| XDH | 7498 |
| XK | 7504 |

TC 5

| | |
|---|---|
| ABCC3 | 8714 |
| AGR2 | 10551 |
| ANXA3 | 306 |
| AP1M2 | 10053 |
| ARHGAP8 | 23779 |
| ATAD4 | 79170 |
| B3GNT1 | 11041 |
| B3GNT3 | 10331 |
| BACE2 | 25825 |
| BIK | 638 |
| C1ORF106 | 55765 |
| CCL20 | 6364 |
| CDCP1 | 64866 |
| CEACAM6 | 4680 |
| CIB1 | 10519 |
| CKMT1B | 1159 |
| CLDN4 | 1364 |
| CLDN7 | 1366 |
| CXCL3 | 2921 |
| EFHD2 | 79180 |
| ELF3 | 1999 |
| ELF4 | 2000 |
| ELMO3 | 79767 |
| EPCAM | 4072 |
| EPHA2 | 1969 |
| EPS8L1 | 54869 |
| ERBB3 | 2065 |
| F2RL1 | 2150 |
| FA2H | 79152 |
| FAM110B | 90362 |
| FERMT1 | 55612 |
| FUT2 | 2524 |
| GALE | 2582 |
| GALNT12 | 79695 |
| GCNT3 | 9245 |
| GJB3 | 2707 |
| GMDS | 2762 |
| GPRC5A | 9052 |
| GPX2 | 2877 |
| GSTP1 | 2950 |
| HK2 | 3099 |
| ITGB4 | 3691 |
| ITPR3 | 3710 |
| JUP | 3728 |
| KCNK1 | 3775 |
| KCNN4 | 3783 |
| KLF5 | 688 |
| KRT18 | 3875 |
| KRT8 | 3856 |
| LAD1 | 3898 |
| LAMB3 | 3914 |
| LAMC2 | 3918 |
| LCN2 | 3934 |
| LGALS4 | 3960 |
| LSR | 51599 |
| MALL | 7851 |
| MAP2K3 | 5606 |
| MAPK13 | 5603 |
| MYH14 | 79784 |
| MYO1E | 4643 |
| NANS | 54187 |
| NQO1 | 1728 |
| PIGR | 5284 |
| PKP3 | 11187 |
| PLEK2 | 26499 |
| PLS1 | 5357 |
| PMM2 | 5373 |
| POF1B | 79983 |
| PPAP2C | 8612 |
| PPARG | 5468 |
| PRSS8 | 5652 |
| QSOX1 | 5768 |
| RAB11FIP1 | 80223 |
| RAB25 | 57111 |
| S100A14 | 57402 |
| S100P | 6286 |
| SDC1 | 6382 |
| SERPINB5 | 5268 |
| SFN | 2810 |
| SLC44A4 | 80736 |
| SMAGP | 57228 |
| SOX9 | 6662 |
| ST14 | 6768 |
| TBC1D13 | 54662 |
| TCEA2 | 6919 |
| TFF1 | 7031 |
| TJP3 | 27134 |
| TMC5 | 79838 |
| TMPRSS2 | 7113 |
| TMPRSS4 | 56649 |
| TRAK1 | 22906 |
| TRPM4 | 54795 |
| TSPAN1 | 10103 |
| TSPAN8 | 7103 |
| TST | 7263 |
| TSTA3 | 7264 |
| VPS37B | 79720 |
| ZC3H12A | 80149 |

TC 6

| | |
|---|---|
| ABCC1 | 4363 |
| ABL2 | 27 |
| ACTB | 60 |
| ACTBL3 | 440915 |
| ADAM17 | 6868 |
| ADH6 | 130 |
| AMIGO2 | 347902 |
| C14ORF105 | 55195 |
| C5 | 727 |
| CFL1 | 1072 |
| CKAP4 | 10970 |
| CRAT | 1384 |
| DPY19L1 | 23333 |
| EPB49 | 2039 |
| EPHX2 | 2053 |
| GAL3ST1 | 9514 |
| HK1 | 3098 |
| MAST3 | 23031 |
| MICB | 4277 |
| PABPC1 | 26986 |
| PAIP2B | 400961 |
| PANX1 | 24145 |
| PPRC1 | 23082 |
| R3HCC1 | 203069 |
| SERPINA6 | 866 |
| SLC20A1 | 6574 |
| TRAM2 | 9697 |
| VTN | 7448 |

TC 7

| | |
|---|---|
| ACCN3 | 9311 |
| AP3B2 | 8120 |
| ATP8A2 | 51761 |
| ATRNL1 | 26033 |
| B3GAT1 | 27087 |
| BAG3 | 9531 |
| BCAM | 4059 |
| BZRAP1 | 9256 |
| C20ORF46 | 55321 |
| CALY | 50632 |
| CAPZB | 832 |
| CLCN4 | 1183 |
| CRMP1 | 1400 |
| CYP46A1 | 10858 |
| DBC1 | 1620 |
| DCX | 1641 |
| DDX25 | 29118 |
| DKFZP434H1419 | 150967 |
| DOCK3 | 1795 |
| DPP6 | 1804 |
| EFNB3 | 1949 |
| ERP44 | 23071 |
| FAM155B | 27112 |
| FAM164C | 79696 |
| FEV | 54738 |
| GNAZ | 2781 |
| GNG4 | 2786 |
| HMP19 | 51617 |
| IQSEC3 | 440073 |
| KCNB1 | 3745 |
| KIAA0408 | 9729 |
| LRP2BP | 55805 |
| LRRTM2 | 26045 |
| MYT1L | 23040 |
| NACAD | 23148 |
| NECAB2 | 54550 |
| NECAP2 | 55707 |
| NPAS3 | 64067 |
| NRXN1 | 9378 |
| NXF2 | 56001 |
| OGDHL | 55753 |
| PAK3 | 5063 |
| PART1 | 25859 |
| PCSK2 | 5126 |
| PPP1R1A | 5502 |
| PTPRT | 11122 |
| RAB26 | 25837 |
| RER1 | 11079 |
| REXO2 | 25996 |
| RUNDC3A | 10900 |
| SCN3B | 55800 |
| SLC8A2 | 6543 |
| SPOCK3 | 50859 |
| STXBP5L | 9515 |
| SYN1 | 6853 |
| TAGLN3 | 29114 |
| TPM4 | 7171 |
| TXNDC5 | 81567 |
| ZNF510 | 22869 |
| ZNF839 | 55778 |

TC 8

| | |
|---|---|
| ABHD8 | 79575 |
| ACTL6B | 51412 |
| ACTR3 | 10096 |
| ADAMTSL2 | 9719 |
| ADCY1 | 107 |
| AGPS | 8540 |
| APBB1 | 322 |
| ATP1A3 | 478 |
| BAIAP3 | 8938 |
| BAZ1A | 11177 |
| BCL10 | 8915 |
| BSN | 8927 |
| C1QL1 | 10882 |
| C3ORF18 | 51161 |
| CACNA1H | 8912 |
| CAMK2B | 816 |
| CCDC6 | 8030 |
| CDK5R2 | 8941 |
| CDR2 | 1039 |
| CHD5 | 26038 |
| COLQ | 8292 |
| CPLX2 | 10814 |
| CRLF3 | 51379 |
| CYFIP1 | 23191 |
| DLG4 | 1742 |
| DTX3 | 196403 |
| EPOR | 2057 |
| EXTL3 | 2137 |
| F10 | 2159 |
| GRIA3 | 2892 |
| GRIK5 | 2901 |
| HIF1A | 3091 |
| HIF3A | 64344 |
| IER5 | 51278 |
| IGF2AS | 51214 |
| KCTD9 | 54793 |
| KLKB1 | 3818 |
| LOC728448 | 728448 |
| LPPR2 | 64748 |
| LRRC23 | 10233 |
| MTDH | 92140 |
| NEURL | 9148 |
| PKD1 | 5310 |
| RAB3A | 5864 |
| RALA | 5898 |
| REEP2 | 51308 |
| REM1 | 28954 |
| RGS12 | 6002 |
| SLC25A24 | 29957 |
| SLK | 9748 |
| SNPH | 9751 |
| SNTA1 | 6640 |
| SNX6 | 58533 |
| SSTR2 | 6752 |
| SYP | 6855 |
| SYT5 | 6861 |
| TMEM123 | 114908 |
| UBE2D1 | 7321 |
| UNC13A | 23025 |
| USP15 | 9958 |
| ZNF217 | 7764 |
| ZNF267 | 10308 |
| ZNF428 | 126299 |
| ZNF446 | 55663 |
| ZNF671 | 79891 |

TC 9

| | |
|---|---|
| ANKMY1 | 51281 |
| AP3S1 | 1176 |
| ARID3B | 10620 |
| ASPH | 444 |
| C14ORF79 | 122616 |
| CAPN10 | 11132 |
| CATSPER2 | 117155 |
| CCDC106 | 29903 |
| CCNJL | 79616 |
| CDC42BPA | 8476 |
| CLINT1 | 9685 |
| CLSTN3 | 9746 |
| CXORF21 | 80231 |
| DKFZP547G183 | 55525 |
| DVL2 | 1856 |
| FLJ13769 | 80079 |
| FLJ14031 | 80089 |
| FXR2 | 9513 |
| GFOD2 | 81577 |
| GLUD1 | 2746 |
| GRIK2 | 2898 |
| KIAA0319 | 9856 |
| KIAA0494 | 9813 |
| KLHL25 | 64410 |
| LTB4R | 1241 |
| MAST2 | 23139 |
| MBD3 | 53615 |
| MED16 | 10025 |
| MED9 | 55090 |
| MGC13053 | 84796 |
| MYO9A | 4649 |
| NARFL | 64428 |
| NRIP2 | 83714 |
| NRXN2 | 9379 |
| NT5DC3 | 51559 |
| NUP188 | 23511 |
| PODXL2 | 50512 |
| POMT2 | 29954 |
| PPFIA3 | 8541 |
| PPP2R5B | 5526 |
| PRKAR1B | 5575 |
| PTDSS2 | 81490 |
| RNF25 | 64320 |
| SEMA3F | 6405 |
| SFI1 | 9814 |
| SGTA | 6449 |
| SOAT1 | 6646 |
| SULT4A1 | 25830 |
| TMEM104 | 54868 |
| TNPO2 | 30000 |
| TRAPPC9 | 83696 |
| TRPC4 | 7223 |
| UEVLD | 55293 |
| WBSCR23 | 80112 |
| WSCD1 | 23302 |
| ZBTB22 | 9278 |
| ZDHHC8P | 150244 |
| ZNF574 | 64763 |
| ZNF76 | 7629 |

TC 10

| | |
|---|---|
| A4GALT | 53947 |
| ABCB11 | 8647 |
| ABCB6 | 10058 |
| ABCB8 | 11194 |
| ABCB9 | 23457 |
| ABCG4 | 64137 |
| ABI1 | 10006 |
| ACADS | 35 |
| ACAP1 | 9744 |
| ACCN1 | 40 |
| ACCN4 | 55515 |
| ACR | 49 |
| ACRV1 | 56 |
| ACSBG1 | 23205 |
| ACSBG2 | 81616 |
| ACTL7A | 10881 |
| ACTL7B | 10880 |
| ACTL8 | 81569 |
| ACTN3 | 89 |
| ACVR1B | 91 |
| ADAM11 | 4185 |
| ADAM18 | 8749 |
| ADAM20 | 8748 |
| ADAM22 | 53616 |
| ADAM29 | 11086 |
| ADAM30 | 11085 |
| ADAM5P | 255926 |
| ADAM7 | 8756 |
| ADAMTS7 | 11173 |
| ADARB2 | 105 |
| ADCK4 | 79934 |
| ADCY10 | 55811 |
| ADCY8 | 114 |
| ADM2 | 79924 |
| ADRA1A | 148 |
| ADRA1B | 147 |
| ADRA1D | 146 |
| ADRA2B | 151 |
| ADRA2C | 152 |
| ADRB3 | 155 |
| ADRBK1 | 156 |
| AEN | 64782 |
| AFF1 | 4299 |
| AFF2 | 2334 |
| AGAP2 | 116986 |
| AGFG2 | 3268 |
| AGRP | 181 |
| AIDA | 64853 |
| AIPL1 | 23746 |
| AIRE | 326 |
| AKAP3 | 10566 |
| AKAP4 | 8852 |
| ALKBH4 | 54784 |
| ALLC | 55821 |
| ALOX12B | 242 |
| ALOX12P2 | 245 |
| ALOX15 | 246 |
| ALOXE3 | 59344 |
| ALPP | 250 |
| ALPPL2 | 251 |
| ALX3 | 257 |
| ALX4 | 60529 |
| AMBN | 258 |
| AMELY | 266 |
| AMHR2 | 269 |
| AMN | 81693 |
| ANGPT4 | 51378 |
| ANK1 | 286 |
| ANKRD2 | 26287 |
| ANKRD53 | 79998 |
| ANP32C | 23520 |
| APBA1 | 320 |
| APC2 | 10297 |
| APOA4 | 337 |
| APOBEC2 | 10930 |
| APOBEC3F | 200316 |
| APOC4 | 346 |
| APOL2 | 23780 |
| APOL5 | 80831 |
| AQP6 | 363 |
| ARAP1 | 116985 |
| ARFRP1 | 10139 |
| ARG1 | 383 |
| ARHGDIG | 398 |
| ARHGEF1 | 9138 |
| ARID5A | 10865 |
| ARL4D | 379 |
| ARMC6 | 93436 |
| ARR3 | 407 |
| ARSF | 416 |
| ART1 | 417 |
| ARVCF | 421 |
| ASB7 | 140460 |
| ASCL3 | 56676 |
| ASIP | 434 |
| ATF5 | 22809 |
| ATF6B | 1388 |
| ATP2A1 | 487 |
| ATP2B2 | 491 |
| ATP2B3 | 492 |
| ATXN2L | 11273 |
| ATXN3L | 92552 |
| ATXN8OS | 6315 |
| AURKC | 6795 |
| AVP | 551 |
| AVPR1A | 552 |
| AVPR1B | 553 |
| B3GALT1 | 8708 |
| B3GNT4 | 79369 |
| B9D2 | 80776 |
| BAI1 | 575 |
| BAZ2A | 11176 |
| BBC3 | 27113 |
| BCL2 | 596 |
| BCL2L10 | 10017 |
| BEGAIN | 57596 |
| BEST1 | 7439 |
| BIRC2 | 329 |
| BMP10 | 27302 |
| BMP15 | 9210 |
| BMP3 | 651 |
| BMP6 | 654 |
| BPY2 | 9083 |
| BRD7P3 | 23629 |
| BRF1 | 2972 |
| BRSK2 | 9024 |
| BTG4 | 54766 |
| BTN2A3 | 54718 |
| BTNL2 | 56244 |
| BZRPL1 | 222642 |
| C10ORF68 | 79741 |
| C10ORF95 | 79946 |
| C11ORF16 | 56673 |
| C11ORF20 | 25858 |
| C11ORF21 | 29125 |
| C14ORF113 | 54792 |
| C14ORF115 | 55237 |
| C14ORF162 | 56936 |
| C14ORF56 | 89919 |
| C15ORF31 | 9593 |
| C15ORF34 | 80072 |
| C15ORF49 | 63969 |
| C16ORF71 | 146562 |
| C17ORF53 | 78995 |
| C17ORF59 | 54785 |
| C17ORF88 | 23591 |
| C19ORF36 | 113177 |
| C19ORF40 | 91442 |
| C19ORF57 | 79173 |
| C19ORF73 | 55150 |
| C1ORF105 | 92346 |
| C1ORF113 | 79729 |
| C1ORF129 | 80133 |
| C1ORF14 | 81626 |
| C1ORF159 | 54991 |
| C1ORF175 | 374977 |
| C1ORF20 | 116492 |
| C1ORF222 | 339457 |
| C1ORF61 | 10485 |
| C1ORF68 | 100129271 |
| C1ORF89 | 79363 |
| C21ORF2 | 755 |
| C21ORF77 | 55264 |
| C22ORF24 | 25775 |
| C22ORF26 | 55267 |
| C22ORF28 | 51493 |
| C22ORF31 | 25770 |
| C22ORF36 | 388886 |
| C2ORF27A | 29798 |
| C2ORF83 | 56918 |
| C3ORF27 | 23434 |
| C3ORF36 | 80111 |
| C6ORF15 | 29113 |
| C6ORF208 | 80069 |
| C6ORF25 | 80739 |
| C6ORF27 | 80737 |
| C6ORF47 | 57827 |
| C6ORF54 | 26236 |
| C7ORF69 | 80099 |
| C8ORF17 | 56988 |
| C8ORF39 | 55472 |
| C8ORF44 | 56260 |
| C9ORF31 | 57000 |
| C9ORF38 | 29044 |
| C9ORF53 | 51198 |
| C9ORF68 | 55064 |
| CA5A | 763 |
| CA5B | 11238 |
| CA6 | 765 |
| CA7 | 766 |
| CABP1 | 9478 |
| CABP2 | 51475 |
| CABP5 | 56344 |
| CACNA1F | 778 |
| CACNA1G | 8913 |
| CACNA1I | 8911 |
| CACNA1S | 779 |
| CACNA2D1 | 781 |
| CACNB1 | 782 |
| CACNB4 | 785 |
| CACNG1 | 786 |
| CACNG2 | 10369 |
| CACNG3 | 10368 |
| CACNG4 | 27092 |
| CACNG5 | 27091 |
| CADM3 | 57863 |
| CADM4 | 199731 |
| CAMK1G | 57172 |
| CAMK2A | 815 |
| CAMKV | 79012 |
| CAMP | 820 |
| CAPN11 | 11131 |
| CARD14 | 79092 |
| CASP10 | 843 |
| CASP2 | 835 |
| CASR | 846 |
| CAV3 | 859 |
| CCBP2 | 1238 |
| CCDC134 | 79879 |
| CCDC19 | 25790 |
| CCDC28B | 79140 |
| CCDC33 | 80125 |
| CCDC40 | 55036 |
| CCDC70 | 83446 |
| CCDC71 | 64925 |
| CCDC85B | 11007 |
| CCDC87 | 55231 |
| CCDC9 | 26093 |
| CCIN | 881 |
| CCKAR | 886 |
| CCL1 | 6346 |
| CCL25 | 6370 |
| CCL27 | 10850 |
| CCR3 | 1232 |
| CCR4 | 1233 |
| CCRN4L | 25819 |
| CCT8L2 | 150160 |
| CD244 | 51744 |
| CD40LG | 959 |
| CD6 | 923 |
| CDC37P1 | 390688 |
| CDH15 | 1013 |
| CDH18 | 1016 |
| CDH22 | 64405 |
| CDH7 | 1005 |
| CDH8 | 1006 |
| CDKL5 | 6792 |
| CDKN2D | 1032 |
| CDRT1 | 374286 |
| CDSN | 1041 |
| CDX4 | 1046 |
| CDY1 | 9085 |
| CEACAM21 | 90273 |
| CEACAM3 | 1084 |
| CEACAM4 | 1089 |
| CEBPE | 1053 |
| CELSR1 | 9620 |
| CEMP1 | 752014 |
| CEND1 | 51286 |
| CER1 | 9350 |
| CES4 | 51716 |
| CETN1 | 1068 |
| CETP | 1071 |
| CHAT | 1103 |
| CHIC2 | 26511 |
| CHRM2 | 1129 |
| CHRM5 | 1133 |
| CHRNA10 | 57053 |
| CHRNA2 | 1135 |
| CHRNA4 | 1137 |
| CHRNA6 | 8973 |
| CHRNB2 | 1141 |
| CHRNB3 | 1142 |
| CHRND | 1144 |
| CHRNE | 1145 |
| CHRNG | 1146 |
| CHST8 | 64377 |
| CIC | 23152 |
| CIITA | 4261 |
| CLCN1 | 1180 |
| CLCN7 | 1186 |
| CLCNKB | 1188 |
| CLDN17 | 26285 |
| CLDN6 | 9074 |
| CLDN9 | 9080 |
| CLEC1B | 51266 |
| CLEC4M | 10332 |
| CLSPN | 63967 |
| CNGB1 | 1258 |
| CNGB3 | 54714 |
| CNPY4 | 245812 |
| CNR1 | 1268 |
| CNR2 | 1269 |
| CNTD2 | 79935 |
| CNTF | 1270 |
| CNTN2 | 6900 |
| COL11A2 | 1302 |
| COL19A1 | 1310 |
| CORO7 | 79585 |
| CPNE6 | 9362 |
| CPNE7 | 27132 |
| CRHR1 | 1394 |
| CRHR2 | 1395 |
| CRISP1 | 167 |
| CRLF2 | 64109 |
| CRNN | 49860 |
| CROCCL2 | 114819 |
| CRTC1 | 23373 |
| CRX | 1406 |
| CRYAA | 1409 |
| CRYBA1 | 1411 |
| CRYBA4 | 1413 |
| CRYBB1 | 1414 |
| CRYBB2P1 | 1416 |
| CRYBB3 | 1417 |
| CRYGA | 1418 |
| CRYGB | 1419 |
| CRYGC | 1420 |
| CSDC2 | 27254 |
| CSF1 | 1435 |
| CSF2 | 1437 |
| CSF3 | 1440 |
| CSH1 | 1442 |
| CSH2 | 1443 |
| CSHL1 | 1444 |
| CSNK1G1 | 53944 |
| CSPG4LYP2 | 84664 |
| CSRP3 | 8048 |
| CST8 | 10047 |
| CTA- | 79640 |
| 216E10.6 | |
| CTDP1 | 9150 |
| CTNNA3 | 29119 |
| CXCR3 | 2833 |
| CXCR5 | 643 |
| CXORF27 | 25763 |
| CYHR1 | 50626 |
| CYLC2 | 1539 |
| CYP11A1 | 1583 |
| CYP11B1 | 1584 |
| CYP11B2 | 1585 |
| CYP2A13 | 1553 |
| CYP2A7P1 | 1550 |
| CYP2D6 | 1565 |
| CYP2F1 | 1572 |
| CYP2W1 | 54905 |
| DAGLA | 747 |
| DAO | 1610 |
| DBH | 1621 |
| DCAKD | 79877 |
| DCC | 1630 |
| DCHS2 | 54798 |
| DDN | 23109 |
| DDX49 | 54555 |
| DDX54 | 79039 |
| DEC1 | 50514 |
| DEFA4 | 1669 |
| DGCR11 | 25786 |
| DGCR14 | 8220 |
| DGCR6L | 85359 |
| DGCR9 | 25787 |
| DHRS12 | 79758 |
| DISC1 | 27185 |
| DKFZP434B2016 | 642780 |
| DKFZP564C196 | 284649 |
| DKFZP566H0824 | 54744 |
| DKKL1 | 27120 |
| DLEC1 | 9940 |
| DLGAP2 | 9228 |
| DLX4 | 1748 |
| DMC1 | 11144 |
| DMWD | 1762 |
| DNAH2 | 146754 |
| DNAH3 | 55567 |
| DNAH6 | 1768 |
| DNAH9 | 1770 |
| DNAI2 | 64446 |
| DNASE1L2 | 1775 |
| DNMT3L | 29947 |
| DNTT | 1791 |
| DOC2A | 8448 |
| DOC2B | 8447 |
| DOHH | 83475 |
| DOK1 | 1796 |
| DPF1 | 8193 |
| DPYSL4 | 10570 |
| DRD2 | 1813 |
| DRD3 | 1814 |
| DRD5 | 1816 |
| DRP2 | 1821 |
| DSC1 | 1823 |
| DSCR4 | 10281 |
| DTNB | 1838 |
| DUS2L | 54920 |
| DUSP13 | 51207 |
| DUSP21 | 63904 |
| DUSP9 | 1852 |
| DUX1 | 26584 |
| DUX4 | 22947 |
| DUX5 | 26581 |
| DYRK1B | 9149 |
| E2F2 | 1870 |
| E2F4 | 1874 |
| EDA2R | 60401 |
| EFNA2 | 1943 |
| EFR3B | 22979 |
| ELAVL3 | 1995 |
| ELSPBP1 | 64100 |
| EML2 | 24139 |
| EMR3 | 84658 |
| EMX1 | 2016 |
| ENTPD2 | 954 |
| EPAG | 10824 |
| EPB41 | 2035 |
| EPB42 | 2038 |
| EPHB4 | 2050 |
| EPN1 | 29924 |
| EPO | 2056 |
| EPX | 8288 |
| ERAF | 51327 |
| ERICH1 | 157697 |
| ESR2 | 2100 |
| ESRRB | 2103 |
| ETV2 | 2116 |
| ETV3 | 2117 |
| ETV7 | 51513 |
| EVX1 | 2128 |
| EXD3 | 54932 |
| EXOC1 | 55763 |
| EXOG | 9941 |
| EXTL1 | 2134 |
| F11 | 2160 |
| FABP2 | 2169 |
| FAM111A | 63901 |
| FAM153A | 285596 |
| FAM182A | 284800 |
| FAM3A | 60343 |
| FAM66D | 100132923 |
| FAM75A7 | 26165 |
| FANCC | 2176 |
| FASLG | 356 |
| FBRS | 64319 |
| FBXL18 | 80028 |
| FBXO24 | 26261 |
| FBXO28 | 23219 |
| FCAR | 2204 |
| FCER2 | 2208 |
| FCN2 | 2220 |
| FETUB | 26998 |
| FEZF2 | 55079 |
| FFAR3 | 2865 |
| FGF16 | 8823 |
| FGF17 | 8822 |
| FGF21 | 26291 |
| FGF23 | 8074 |
| FGF3 | 2248 |
| FGF6 | 2251 |
| FKBP6 | 8468 |
| FLJ00049 | 645372 |
| FLJ10232 | 55099 |
| FLJ11710 | 79904 |
| FLJ11827 | 80163 |
| FLJ12547 | 80058 |
| FLJ12616 | 196707 |
| FLJ13310 | 80188 |
| FLJ14100 | 80093 |
| FLJ20712 | 55025 |
| FLJ22596 | 80156 |
| FLJ23185 | 80126 |
| FLRT1 | 23769 |
| FN3K | 64122 |
| FNDC8 | 54752 |
| FOLR3 | 2352 |
| FOXB1 | 27023 |
| FOXC2 | 2303 |
| FOXD4 | 2298 |
| FOXE3 | 2301 |
| FOXH1 | 8928 |
| FOXJ1 | 2302 |
| FOXL1 | 2300 |
| FOXN1 | 8456 |
| FOXO4 | 4303 |
| FOXP3 | 50943 |
| FRMD1 | 79981 |
| FRMPD1 | 22844 |
| FRMPD4 | 9758 |
| FRS3 | 10817 |
| FSCN3 | 29999 |
| FSHB | 2488 |
| FSHR | 2492 |
| FSTL4 | 23105 |
| FUT7 | 2529 |
| FUZ | 80199 |
| FXYD7 | 53822 |
| FZD9 | 8326 |
| FZR1 | 51343 |
| G6PC2 | 57818 |
| GABARAPL3 | 23766 |
| GABRA3 | 2556 |
| GABRA6 | 2559 |
| GABRQ | 55879 |
| GABRR2 | 2570 |
| GALNT8 | 26290 |
| GATA1 | 2623 |
| GBX1 | 2636 |
| GBX2 | 2637 |
| GCGR | 2642 |
| GCK | 2645 |
| GCM1 | 8521 |
| GCNT4 | 51301 |
| GDAP1L1 | 78997 |
| GDF11 | 10220 |
| GDF2 | 2658 |
| GDF3 | 9573 |
| GDF5 | 8200 |
| GFI1 | 2672 |
| GFRA2 | 2675 |
| GFRA4 | 64096 |
| GGTLC2 | 91227 |
| GH2 | 2689 |
| GHRHR | 2692 |
| GHSR | 2693 |
| GIPR | 2696 |
| GIT1 | 28964 |
| GJA3 | 2700 |
| GJA8 | 2703 |
| GJB4 | 127534 |
| GJC2 | 57165 |
| GJD2 | 57369 |
| GLI1 | 2735 |
| GLP1R | 2740 |
| GLP2R | 9340 |
| GLRA1 | 2741 |
| GLRA2 | 2742 |
| GLRA3 | 8001 |
| GML | 2765 |
| GNAO1 | 2775 |
| GNAT1 | 2779 |
| GNB3 | 2784 |
| GNG13 | 51764 |
| GNG3 | 2785 |
| GNG7 | 2788 |
| GNL3LP | 80060 |
| GNMT | 27232 |
| GNRH2 | 2797 |
| GNRHR | 2798 |
| GP1BA | 2811 |
| GP1BB | 2812 |
| GP5 | 2814 |
| GP9 | 2815 |
| GPR12 | 2835 |
| GPR132 | 29933 |
| GPR135 | 64582 |
| GPR144 | 347088 |
| GPR162 | 27239 |
| GPR17 | 2840 |
| GPR182 | 11318 |
| GPR21 | 2844 |
| GPR22 | 2845 |
| GPR25 | 2848 |
| GPR3 | 2827 |
| GPR31 | 2853 |
| GPR32 | 2854 |
| GPR44 | 11251 |
| GPR45 | 11250 |
| GPR50 | 9248 |
| GPR52 | 9293 |
| GPR63 | 81491 |
| GPR75 | 10936 |
| GPR77 | 27202 |
| GPR97 | 222487 |
| GPRC5D | 55507 |
| GPX5 | 2880 |
| GRAP | 10750 |
| GRAP2 | 9402 |
| GREB1 | 9687 |
| GRIA1 | 2890 |
| GRID2 | 2895 |
| GRIK1 | 2897 |
| GRIK3 | 2899 |
| GRIN1 | 2902 |
| GRIN2B | 2904 |
| GRIN2C | 2905 |
| GRIP1 | 23426 |
| GRIP2 | 80852 |
| GRK1 | 6011 |
| GRM1 | 2911 |
| GRM2 | 2912 |
| GRM4 | 2914 |
| GRM5 | 2915 |
| GRPR | 2925 |
| GRRP1 | 79927 |
| GRWD1 | 83743 |
| GSG1 | 83445 |
| GSK3A | 2931 |
| GSTA3 | 2940 |
| GSTTP1 | 25774 |
| GTPBP1 | 9567 |
| GUCA1A | 2978 |
| GUCA1B | 2979 |
| GUCA2A | 2980 |
| GUCY2D | 3000 |
| GUCY2F | 2986 |
| GYPA | 2993 |
| GYPB | 2994 |
| GZMM | 3004 |
| H2AFB3 | 83740 |
| HAB1 | 55547 |
| HAND2 | 9464 |
| HAP1 | 9001 |
| HAPLN2 | 60484 |
| HBBP1 | 3044 |
| HBE1 | 3046 |
| HBQ1 | 3049 |
| HCFC1 | 3054 |
| HCG2P7 | 80867 |
| HCG9 | 10255 |
| HCG_1732469 | 729164 |
| HCN2 | 610 |
| HCRT | 3060 |
| HCRTR1 | 3061 |
| HCRTR2 | 3062 |
| HDAC11 | 79885 |
| HDAC6 | 10013 |
| HDAC7 | 51564 |
| HECW1 | 23072 |
| HES2 | 54626 |
| HGC6.3 | 100128124 |
| HGFAC | 3083 |
| HHLA1 | 10086 |
| HIST1H1A | 3024 |
| HIST1H1B | 3009 |
| HIST1H1D | 3007 |
| HIST1H1E | 3008 |
| HIST1H1T | 3010 |
| HIST1H2AK | 8330 |
| HIST1H2BL | 8340 |
| HIST1H3I | 8354 |
| HIST1H3J | 8356 |
| HIST1H4G | 8369 |
| HIST1H4I | 8294 |
| HMGN4 | 10473 |
| HMX1 | 3166 |
| HNRNPUL2 | 221092 |
| HOXA6 | 3203 |
| HOXB1 | 3211 |
| HOXB8 | 3218 |
| HOXC8 | 3224 |
| HOXD12 | 3238 |
| HOXD3 | 3232 |
| HPCA | 3208 |
| HPCAL4 | 51440 |
| HPSE2 | 60495 |
| HRASLS2 | 54979 |
| HRC | 3270 |
| HRH2 | 3274 |
| HRH3 | 11255 |
| HRK | 8739 |
| HS1BP3 | 64342 |
| HS6ST1 | 9394 |
| HSD17B14 | 51171 |
| HSF4 | 3299 |
| HSPA1L | 3305 |
| HSPC072 | 29075 |
| HTR1A | 3350 |
| HTR1B | 3351 |
| HTR1D | 3352 |
| HTR1E | 3354 |
| HTR3A | 3359 |
| HTR3B | 9177 |
| HTR4 | 3360 |
| HTR5A | 3361 |
| HTR6 | 3362 |
| HTR7 | 3363 |
| HTR7P | 93164 |
| HUMBINDC | 29892 |
| HUNK | 30811 |
| HUWE1 | 10075 |
| HYDIN | 54768 |
| ICAM5 | 7087 |
| IFNA1 | 3439 |
| IFNA16 | 3449 |
| IFNA17 | 3451 |
| IFNA21 | 3452 |
| IFNA4 | 3441 |
| IFNA5 | 3442 |
| IFNA7 | 3444 |
| IFNB1 | 3456 |
| IFNW1 | 3467 |
| IGFALS | 3483 |
| IGSF9B | 22997 |
| IL12RB1 | 3594 |
| IL13 | 3596 |
| IL17A | 3605 |
| IL17B | 27190 |
| IL19 | 29949 |
| IL1F6 | 27179 |
| IL1RAPL1 | 11141 |
| IL1RAPL2 | 26280 |
| IL1RL2 | 8808 |
| IL21 | 59067 |
| IL25 | 64806 |
| IL3 | 3562 |
| IL4 | 3565 |
| IL5 | 3567 |
| IL5RA | 3568 |
| IL9R | 3581 |
| IMPG2 | 50939 |
| INE1 | 8552 |
| INSL3 | 3640 |
| INSL6 | 11172 |
| INSRR | 3645 |
| IQCC | 55721 |
| IQSEC2 | 23096 |
| IRGC | 56269 |
| IRS4 | 8471 |
| ITGA2B | 3674 |
| ITGB1BP3 | 27231 |
| ITGB3 | 3690 |
| JAK3 | 3718 |
| JPH3 | 57338 |
| KANK1 | 23189 |
| KCNA10 | 3744 |
| KCNA2 | 3737 |
| KCNA3 | 3738 |
| KCNA6 | 3742 |
| KCNAB3 | 9196 |
| KCNB2 | 9312 |
| KCNC1 | 3746 |
| KCNC2 | 3747 |
| KCNE1 | 3753 |
| KCNE1L | 23630 |
| KCNG1 | 3755 |
| KCNH1 | 3756 |
| KCNH4 | 23415 |
| KCNH6 | 81033 |
| KCNIP2 | 30819 |
| KCNJ10 | 3766 |
| KCNJ12 | 3768 |
| KCNJ14 | 3770 |
| KCNJ4 | 3761 |
| KCNJ5 | 3762 |
| KCNJ9 | 3765 |
| KCNK10 | 54207 |
| KCNK7 | 10089 |
| KCNN1 | 3780 |
| KCNQ1DN | 55539 |
| KCNQ2 | 3785 |
| KCNQ3 | 3786 |
| KCNQ4 | 9132 |
| KCNS1 | 3787 |
| KCNV2 | 169522 |
| KCTD17 | 79734 |
| KEL | 3792 |
| KHDRBS2 | 202559 |
| KIAA0509 | 57242 |
| KIAA1045 | 23349 |
| KIAA1614 | 57710 |
| KIAA1654 | 85368 |
| KIAA1655 | 85370 |
| KIAA1661 | 85375 |
| KIAA1751 | 85452 |
| KIF24 | 347240 |
| KIF25 | 3834 |
| KIR2DL1 | 3802 |
| KIR2DL2 | 3803 |
| KIR2DL3 | 3804 |
| KIR2DL4 | 3805 |
| KIR2DL5A | 57292 |
| KIR2DS1 | 3806 |
| KIR2DS3 | 3808 |
| KIR2DS4 | 3809 |
| KIR2DS5 | 3810 |
| KIR3DL1 | 3811 |
| KIR3DL3 | 115653 |
| KIR3DX1 | 90011 |
| KIRREL | 55243 |
| KISS1 | 3814 |
| KLF1 | 10661 |
| KLF15 | 28999 |
| KLHL1 | 57626 |
| KLHL35 | 283212 |
| KLK13 | 26085 |
| KLK14 | 43847 |
| KLK15 | 55554 |
| KREMEN2 | 79412 |
| KRT1 | 3848 |
| KRT18P50 | 442236 |
| KRT19P2 | 160313 |
| KRT2 | 3849 |
| KRT3 | 3850 |
| KRT31 | 3881 |
| KRT32 | 3882 |
| KRT33B | 3884 |
| KRT35 | 3886 |
| KRT75 | 9119 |
| KRT76 | 51350 |
| KRT83 | 3889 |
| KRT84 | 3890 |
| KRT85 | 3891 |
| KRT9 | 3857 |
| KRTAP1-1 | 81851 |
| KRTAP1-3 | 81850 |
| KRTAP2-4 | 85294 |
| KRTAP5-9 | 3846 |
| L3MBTL | 26013 |
| LAMB4 | 22798 |
| LARGE | 9215 |
| LCE2B | 26239 |
| LDB3 | 11155 |
| LECT1 | 11061 |
| LENEP | 55891 |
| LHB | 3972 |
| LHX3 | 8022 |
| LHX5 | 64211 |
| LILRA1 | 11024 |
| LILRA3 | 11026 |
| LILRA4 | 23547 |
| LILRA5 | 353514 |
| LILRP2 | 79166 |
| LIM2 | 3982 |
| LIMK1 | 3984 |
| LIPE | 3991 |
| LMAN1L | 79748 |
| LMTK2 | 22853 |
| LMX1B | 4010 |
| LOC100093698 | 100093698 |
| LOC100128008 | 100128008 |
| LOC100128570 | 100128570 |
| LOC100128640 | 100128640 |
| LOC100129015 | 100129015 |
| LOC100129500 | 100129500 |
| LOC100129502 | 100129502 |
| LOC100129503 | 100129503 |
| LOC100129624 | 100129624 |
| LOC100130134 | 100130134 |
| LOC100130354 | 100130354 |
| LOC100130955 | 100130955 |
| LOC100131298 | 100131298 |
| LOC100131509 | 100131509 |
| LOC100131532 | 100131532 |
| LOC100131825 | 100131825 |
| LOC100133724 | 100133724 |
| LOC100134128 | 100134128 |
| LOC100134498 | 100134498 |
| LOC145678 | 145678 |
| LOC145899 | 145899 |
| LOC147343 | 147343 |
| LOC157627 | 157627 |
| LOC1720 | 1720 |
| LOC196993 | 196993 |
| LOC220077 | 220077 |
| LOC26102 | 26102 |
| LOC29034 | 29034 |
| LOC390561 | 390561 |
| LOC399904 | 399904 |
| LOC440366 | 440366 |
| LOC440792 | 440792 |
| LOC441601 | 441601 |
| LOC442421 | 442421 |
| LOC442715 | 442715 |
| LOC51190 | 51190 |
| LOC541469 | 541469 |
| LOC57399 | 57399 |
| LOC642131 | 642131 |
| LOC644450 | 644450 |
| LOC646934 | 646934 |
| LOC649853 | 649853 |
| LOC652147 | 652147 |
| LOC727842 | 727842 |
| LOC728361 | 728361 |
| LOC728564 | 728564 |
| LOC729799 | 729799 |
| LOC729991- | 4207 |
| MEF2B | |
| LOC730227 | 730227 |
| LOC79999 | 79999 |
| LOC80054 | 80054 |
| LOC90586 | 90586 |
| LOC91316 | 91316 |
| LOR | 4014 |
| LPAL2 | 80350 |
| LPO | 4025 |
| LRCH4 | 4034 |
| LRIT1 | 26103 |
| LRRC3 | 81543 |
| LRRC50 | 123872 |
| LRRC68 | 284352 |
| LRTM1 | 57408 |
| LSM14B | 149986 |
| LTA | 4049 |
| LTB4R2 | 56413 |
| LTK | 4058 |
| LUZP4 | 51213 |
| LZTS1 | 11178 |
| MADCAM1 | 8174 |
| MAG | 4099 |
| MAGEB3 | 4114 |
| MAGEC2 | 51438 |
| MAGEC3 | 139081 |
| MAP2K7 | 5609 |
| MAP3K10 | 4294 |
| MAPK11 | 5600 |
| MAPK4 | 5596 |
| MAPK8IP1 | 9479 |
| MAPK8IP2 | 23542 |
| MAPK8IP3 | 23162 |
| MASP1 | 5648 |
| MASP2 | 10747 |
| MATK | 4145 |
| MATN1 | 4146 |
| MATN4 | 8785 |
| MBD2 | 8932 |
| MBD4 | 8930 |
| MBL1P1 | 8512 |
| MC1R | 4157 |
| MC5R | 4161 |
| MDFI | 4188 |
| MDS1 | 4197 |
| MEF2D | 4209 |
| MEGF8 | 1954 |
| MEPE | 56955 |
| MFSD7 | 84179 |
| MGAT3 | 4248 |
| MGAT5 | 4249 |
| MGC2889 | 84789 |
| MGC3771 | 81854 |
| MGC4294 | 79160 |
| MGC51338 | 388358 |
| MGC5566 | 79015 |
| MIIP | 60672 |
| MIP | 4284 |
| MKRN3 | 7681 |
| MLL4 | 9757 |
| MLN | 4295 |
| MLXIPL | 51085 |
| MMP17 | 4326 |
| MMP24 | 10893 |
| MMP25 | 64386 |
| MMP26 | 56547 |
| MOBP | 4336 |
| MORN1 | 79906 |
| MOS | 4342 |
| MPL | 4352 |
| MPP3 | 4356 |
| MPPED1 | 758 |
| MPZ | 4359 |
| MRM1 | 79922 |
| MS4A5 | 64232 |
| MSI1 | 4440 |
| MTHFS | 10588 |
| MTMR7 | 9108 |
| MTMR8 | 55613 |
| MTNR1B | 4544 |
| MTSS1L | 92154 |
| MUC8 | 4590 |
| MUSK | 4593 |
| MVD | 4597 |
| MVK | 4598 |
| MYBPC3 | 4607 |
| MYBPH | 4608 |
| MYCNOS | 10408 |
| MYF5 | 4617 |
| MYH13 | 8735 |
| MYH15 | 22989 |
| MYH6 | 4624 |
| MYL10 | 93408 |
| MYL3 | 4634 |
| MYL7 | 58498 |
| MYO15A | 51168 |
| MYO16 | 23026 |
| MYO3A | 53904 |
| MYO7A | 4647 |
| MYO7B | 4648 |
| MYOD1 | 4654 |
| MYOG | 4656 |
| MYOZ1 | 58529 |
| NBR2 | 10230 |
| NCAPH2 | 29781 |
| NCKIPSD | 51517 |
| NCOR2 | 9612 |
| NCR1 | 9437 |
| NCR2 | 9436 |
| NCR3 | 259197 |
| NCRNA00105 | 80161 |
| NDOR1 | 27158 |
| NDST3 | 9348 |
| NENF | 29937 |
| NEU2 | 4759 |
| NEU3 | 10825 |
| NEUROD2 | 4761 |
| NEUROD4 | 58158 |
| NEUROD6 | 63974 |
| NEUROG1 | 4762 |
| NEUROG2 | 63973 |
| NEUROG3 | 50674 |
| NFKBIL1 | 4795 |
| NFKBIL2 | 4796 |
| NGB | 58157 |
| NGF | 4803 |
| NHLH2 | 4808 |
| NKX2-5 | 1482 |
| NKX2-8 | 26257 |
| NKX3-1 | 4824 |
| NLGN3 | 54413 |
| NLRP3 | 114548 |
| NMUR1 | 10316 |
| NOS1 | 4842 |
| NOVA2 | 4858 |
| NOX5 | 79400 |
| NPAS1 | 4861 |
| NPBWR2 | 2832 |
| NPFFR1 | 64106 |
| NPHS1 | 4868 |
| NPPA | 4878 |
| NPVF | 64111 |
| NPY2R | 4887 |
| NR2E3 | 10002 |
| NR2F6 | 2063 |
| NR5A1 | 2516 |
| NR6A1 | 2649 |
| NRL | 4901 |
| NT5C | 30833 |
| NT5M | 56953 |
| NTN3 | 4917 |
| NTRK1 | 4914 |
| NTRK3 | 4916 |
| NTSR2 | 23620 |
| NUBP2 | 10101 |
| NXPH3 | 11248 |
| NYX | 60506 |
| OAZ3 | 51686 |
| OCLM | 10896 |
| OCM2 | 4951 |
| ODF1 | 4956 |
| OGFR | 11054 |
| OLIG2 | 10215 |
| OMP | 4975 |
| OPCML | 4978 |
| OPN1MW | 2652 |
| OPN1SW | 611 |
| OPRD1 | 4985 |
| OPRL1 | 4987 |
| OPRM1 | 4988 |
| OR10C1 | 442194 |
| OR10H1 | 26539 |
| OR10H2 | 26538 |
| OR10H3 | 26532 |
| OR10J1 | 26476 |
| OR11A1 | 26531 |
| OR12D2 | 26529 |
| OR1A1 | 8383 |
| OR1A2 | 26189 |
| OR1D2 | 4991 |
| OR1D4 | 8385 |
| OR1E1 | 8387 |
| OR1F1 | 4992 |
| OR1F2P | 26184 |
| OR1G1 | 8390 |
| OR2C1 | 4993 |
| OR2F1 | 26211 |
| OR2H1 | 26716 |
| OR2H2 | 7932 |
| OR2J2 | 26707 |
| OR2J3 | 442186 |
| OR3A1 | 4994 |
| OR3A2 | 4995 |
| OR3A3 | 8392 |
| OR52A1 | 23538 |
| OR7A10 | 390892 |
| OR7C1 | 26664 |
| OR7C2 | 26658 |
| OR7E19P | 26651 |
| OR7E87P | 8586 |
| OSBP2 | 23762 |
| OSBPL7 | 114881 |
| OSGIN1 | 29948 |
| OTOF | 9381 |
| OTOR | 56914 |
| OXCT2 | 64064 |
| P2RX2 | 22953 |
| P2RX6 | 9127 |
| P2RY4 | 5030 |
| PACSIN3 | 29763 |
| PADI4 | 23569 |
| PAGE1 | 8712 |
| PAK2 | 5062 |
| PAOX | 196743 |
| PAPPA2 | 60676 |
| PARD6A | 50855 |
| PARK2 | 5071 |
| PAX5 | 5079 |
| PAX7 | 5081 |
| PAX8 | 7849 |
| PBOV1 | 59351 |
| PBX2 | 5089 |
| PCDH1 | 5097 |
| PCDHA10 | 56139 |
| PCDHA2 | 56146 |
| PCDHA3 | 56145 |
| PCDHA5 | 56143 |
| PCDHB1 | 29930 |
| PCDHB17 | 54661 |
| PCDHGA1 | 56114 |
| PCDHGA3 | 56112 |
| PCDHGA9 | 56107 |
| PCDHGB5 | 56101 |
| PDCD1 | 5133 |
| PDE1B | 5153 |
| PDE4A | 5141 |
| PDE6A | 5145 |
| PDE6G | 5148 |
| PDE6H | 5149 |
| PDHA2 | 5161 |
| PDIA2 | 64714 |
| PDX1 | 3651 |
| PDYN | 5173 |
| PDZD7 | 79955 |
| PGK2 | 5232 |
| PGLYRP1 | 8993 |
| PHF7 | 51533 |
| PHKG1 | 5260 |
| PHLDB1 | 23187 |
| PHOX2A | 401 |
| PICK1 | 9463 |
| PIGQ | 9091 |
| PIK3R2 | 5296 |
| PIK3R4 | 30849 |
| PIN1L | 5301 |
| PITX3 | 5309 |
| PIWIL2 | 55124 |
| PKLR | 5313 |
| PLA2G2E | 30814 |
| PLA2G2F | 64600 |
| PLA2G3 | 50487 |
| PLAC4 | 191585 |
| PLCD1 | 5333 |
| PLCH2 | 9651 |
| PLEKHB1 | 58473 |
| PLEKHG3 | 26030 |
| PLEKHM1 | 9842 |
| PLSCR2 | 57047 |
| PMFBP1 | 83449 |
| PMS2L4 | 5382 |
| PNMA3 | 29944 |
| PNPLA2 | 57104 |
| POFUT2 | 23275 |
| POL3S | 339105 |
| POLR2A | 5430 |
| POM121L1P | 25812 |
| POM121L2 | 94026 |
| POMC | 5443 |
| POU2F2 | 5452 |
| POU3F1 | 5453 |
| POU3F3 | 5455 |
| POU3F4 | 5456 |
| POU6F1 | 5463 |
| POU6F2 | 11281 |
| PPAN | 56342 |
| PPBPL2 | 10895 |
| PPIL2 | 23759 |
| PPIL6 | 285755 |
| PPP1R2P9 | 80316 |
| PPP2CA | 5515 |
| PPP3CA | 5530 |
| PPY2 | 23614 |
| PPYR1 | 5540 |
| PQLC2 | 54896 |
| PRAMEF1 | 65121 |
| PRAMEF10 | 343071 |
| PRAMEF11 | 440560 |
| PRAMEF12 | 390999 |
| PRB1 | 5542 |
| PRDM11 | 56981 |
| PRDM12 | 59335 |
| PRDM14 | 63978 |
| PRDM5 | 11107 |
| PRDM8 | 56978 |
| PRDM9 | 56979 |
| PREX2 | 80243 |
| PRG3 | 10394 |
| PRKACG | 5568 |
| PRKCG | 5582 |
| PRL | 5617 |
| PRLH | 51052 |
| PRM1 | 5619 |
| PRM2 | 5620 |
| PRO1768 | 29018 |
| PRO1880 | 29023 |
| PRO2958 | 100128329 |
| PROP1 | 5626 |
| PRPH2 | 5961 |
| PRPS1L1 | 221823 |
| PRRG3 | 79057 |
| PRTN3 | 5657 |
| PRX | 57716 |
| PRY | 9081 |
| PSD | 5662 |
| PSG11 | 5680 |
| PSPN | 5623 |
| PTAFR | 5724 |
| PTCH2 | 8643 |
| PTCRA | 171558 |
| PTGER1 | 5731 |
| PTMS | 5763 |
| PTPN1 | 5770 |
| PTPRS | 5802 |
| PVRL1 | 5818 |
| PVT1 | 5820 |
| PYGO1 | 26108 |
| PYY2 | 23615 |
| PZP | 5858 |
| QPCTL | 54814 |
| RAB3IL1 | 5866 |
| RABEP2 | 79874 |
| RANBP3 | 8498 |
| RAP1B | 5908 |
| RARG | 5916 |
| RASGRF1 | 5923 |
| RASL10A | 10633 |
| RAX | 30062 |
| RB1 | 5925 |
| RBBP9 | 10741 |
| RBMXL2 | 27288 |
| RBMY1A1 | 5940 |
| RBMY2FP | 159162 |
| RBP3 | 5949 |
| RBPJL | 11317 |
| RCE1 | 9986 |
| RCVRN | 5957 |
| RDH16 | 8608 |
| RECQL4 | 9401 |
| RECQL5 | 9400 |
| REST | 5978 |
| RGR | 5995 |
| RGS11 | 8786 |
| RGS6 | 9628 |
| RGSL1 | 353299 |
| RHAG | 6005 |
| RHBDD3 | 25807 |
| RHCE | 6006 |
| RHD | 6007 |
| RHO | 6010 |
| RIBC2 | 26150 |
| RIMS1 | 22999 |
| RIN1 | 9610 |
| RIT2 | 6014 |
| RLBP1 | 6017 |
| RMND5B | 64777 |
| RNASE3 | 6037 |
| RNF121 | 55298 |
| RNF122 | 79845 |
| RNF167 | 26001 |
| RNF17 | 56163 |
| ROM1 | 6094 |
| RP11- | 647288 |
| 159J2.1 | |
| RPGRIP1 | 57096 |
| RPL23AP53 | 644128 |
| RPL3L | 6123 |
| RPS6KA6 | 27330 |
| RPS6KB2 | 6199 |
| RREB1 | 6239 |
| RRH | 10692 |
| RRP1 | 8568 |
| RS1 | 6247 |
| RSHL1 | 81492 |
| RTDR1 | 27156 |
| RTEL1 | 51750 |
| RXFP3 | 51289 |
| S100A5 | 6276 |
| S1PR2 | 9294 |
| SAA3P | 6290 |
| SAG | 6295 |
| SAGE1 | 55511 |
| SAMD14 | 201191 |
| SARDH | 1757 |
| SCAND2 | 54581 |
| SCN10A | 6336 |
| SCN4A | 6329 |
| SCN8A | 6334 |
| SCNN1A | 6337 |
| SCNN1D | 6339 |
| SCT | 6343 |
| SDK2 | 54549 |
| SEC14L3 | 266629 |
| SEMA3B | 7869 |
| SEMA4G | 57715 |
| SEMA6C | 10500 |
| SEMA7A | 8482 |
| SERGEF | 26297 |
| SERPINA2 | 390502 |
| SERPINB10 | 5273 |
| SERPINB13 | 5275 |
| SETD1A | 9739 |
| SH2B1 | 25970 |
| SH3BP1 | 23616 |
| SHANK1 | 50944 |
| SHARPIN | 81858 |
| SHBG | 6462 |
| SHH | 6469 |
| SHOC2 | 8036 |
| SHOX | 6473 |
| SIGLEC5 | 8778 |
| SIGLEC8 | 27181 |
| SIGLEC9 | 27180 |
| SIRPB1 | 10326 |
| SIRT2 | 22933 |
| SIRT5 | 23408 |
| SIX6 | 4990 |
| SLC12A3 | 6559 |
| SLC12A4 | 6560 |
| SLC12A5 | 57468 |
| SLC13A3 | 64849 |
| SLC13A4 | 26266 |
| SLC14A2 | 8170 |
| SLC16A8 | 23539 |
| SLC17A7 | 57030 |
| SLC18A3 | 6572 |
| SLC1A6 | 6511 |
| SLC1A7 | 6512 |
| SLC22A13 | 9390 |
| SLC22A14 | 9389 |
| SLC22A6 | 9356 |
| SLC22A8 | 9376 |
| SLC24A2 | 25769 |
| SLC26A1 | 10861 |
| SLC2A4 | 6517 |
| SLC30A3 | 7781 |
| SLC38A3 | 10991 |
| SLC39A9 | 55334 |
| SLC5A2 | 6524 |
| SLC5A5 | 6528 |
| SLC6A11 | 6538 |
| SLC6A2 | 6530 |
| SLC6A5 | 9152 |
| SLC7A10 | 56301 |
| SLC7A4 | 6545 |
| SLC9A3 | 6550 |
| SLC9A5 | 6553 |
| SLC9A7 | 84679 |
| SLCO5A1 | 81796 |
| SLIT1 | 6585 |
| SLMO1 | 10650 |
| SLURP1 | 57152 |
| SMAD5OS | 9597 |
| SMAD6 | 4091 |
| SMCP | 4184 |
| SMR3B | 10879 |
| SNAPC2 | 6618 |
| SNCB | 6620 |
| SNX26 | 115703 |
| SOX21 | 11166 |
| SOX5 | 6660 |
| SP3P | 160824 |
| SPAG11A | 653423 |
| SPAG11B | 10407 |
| SPAG8 | 26206 |
| SPAM1 | 6677 |
| SPANXA1 | 30014 |
| SPANXC | 64663 |
| SPEF1 | 25876 |
| SPINT3 | 10816 |
| SPN | 6693 |
| SPTB | 6710 |
| SPTBN4 | 57731 |
| SPTBN5 | 51332 |
| SRC | 6714 |
| SRD5A2 | 6716 |
| SRPK3 | 26576 |
| SRY | 6736 |
| SSTR3 | 6753 |
| SSTR4 | 6754 |
| SSX1 | 6756 |
| SSX3 | 10214 |
| SSX5 | 6758 |
| ST3GAL2 | 6483 |
| ST3GAL4 | 6484 |
| STAB2 | 55576 |
| STARD3 | 10948 |
| STK11 | 6794 |
| STMN4 | 81551 |
| STXBP3 | 6814 |
| SYCP1 | 6847 |
| SYMPK | 8189 |
| SYN3 | 8224 |
| SYT12 | 91683 |
| SYT2 | 127833 |
| TAAR5 | 9038 |
| TACR1 | 6869 |
| TACR3 | 6870 |
| TACSTD2 | 4070 |
| TADA3L | 10474 |
| TAF1 | 6872 |
| TAS2R13 | 50838 |
| TAS2R7 | 50837 |
| TAS2R9 | 50835 |
| TBC1D29 | 26083 |
| TBKBP1 | 9755 |
| TBL1Y | 90665 |
| TBR1 | 10716 |
| TBX10 | 347853 |
| TBX4 | 9496 |
| TBX6 | 6911 |
| TBXA2R | 6915 |
| TCAP | 8557 |
| TCEB1P3 | 644540 |
| TCEB3B | 51224 |
| TCF15 | 6939 |
| TCL6 | 27004 |
| TCP10 | 6953 |
| TCTN2 | 79867 |
| TECTA | 7007 |
| TERT | 7015 |
| TEX13A | 56157 |
| TEX13B | 56156 |
| TEX28 | 1527 |
| TFAP4 | 7023 |
| TFDP3 | 51270 |
| TG | 7038 |
| TGM3 | 7053 |
| TGM4 | 7047 |
| TGM5 | 9333 |
| THAP3 | 90326 |
| THEG | 51298 |
| THRA | 7067 |
| TLE6 | 79816 |
| TLL2 | 7093 |
| TLR6 | 10333 |
| TLX2 | 3196 |
| TLX3 | 30012 |
| TM7SF4 | 81501 |
| TMEM121 | 80757 |
| TMEM59L | 25789 |
| TMPRSS5 | 80975 |
| TMSB4Y | 9087 |
| TNFRSF10C | 8794 |
| TNFRSF13B | 23495 |
| TNFRSF4 | 7293 |
| TNK2 | 10188 |
| TNNI1 | 7135 |
| TNP1 | 7141 |
| TNP2 | 7142 |
| TNR | 7143 |
| TNRC4 | 11189 |
| TNXB | 7148 |
| TP53AIP1 | 63970 |
| TP53TG5 | 27296 |
| TP73 | 7161 |
| TPSD1 | 23430 |
| TRAF2 | 7186 |
| TRBV10-2 | 28584 |
| TRBV7-8 | 28590 |
| TREML2 | 79865 |
| TRGV3 | 6976 |
| TRIM10 | 10107 |
| TRIM17 | 51127 |
| TRIM3 | 10612 |
| TRIM62 | 55223 |
| TRMT2A | 27037 |
| TRMT61A | 115708 |
| TRMU | 55687 |
| TRPC7 | 57113 |
| TRPM1 | 4308 |
| TRPV1 | 7442 |
| TRPV5 | 56302 |
| TRPV6 | 55503 |
| TSC22D2 | 9819 |
| TSC22D4 | 81628 |
| TSKS | 60385 |
| TSNAXIP1 | 55815 |
| TSP50 | 29122 |
| TSPY1 | 7258 |
| TSSK1A | 23752 |
| TSSK2 | 23617 |
| TTC22 | 55001 |
| TTC38 | 55020 |
| TTTY1 | 50858 |
| TTTY2 | 60439 |
| TTTY9A | 83864 |
| TUBA8 | 51807 |
| TUBB4Q | 56604 |
| TULP1 | 7287 |
| TULP2 | 7288 |
| TUT1 | 64852 |
| TWF2 | 11344 |
| TXNRD2 | 10587 |
| UBQLN3 | 50613 |
| UBTF | 7343 |
| UCP1 | 7350 |
| UCP3 | 7352 |
| UNC119 | 9094 |
| USP2 | 9099 |
| USP22 | 23326 |
| USP27X | 389856 |
| USP29 | 57663 |
| USP5 | 8078 |
| UTF1 | 8433 |
| VCX2 | 51480 |
| VCY | 9084 |
| VENTX | 27287 |
| VENTXP1 | 139538 |
| VIPR2 | 7434 |
| VN1R1 | 57191 |
| VNN3 | 55350 |
| VPS33A | 65082 |
| WAPAL | 23063 |
| WDR25 | 79446 |
| WDR62 | 284403 |
| WNT1 | 7471 |
| WNT10B | 7480 |
| WNT6 | 7475 |
| WNT7B | 7477 |
| WNT8B | 7479 |
| WSCD2 | 9671 |
| XCR1 | 2829 |
| XKRY | 9082 |
| XPNPEP2 | 7512 |
| YSK4 | 80122 |
| YY2 | 404281 |
| ZBTB32 | 27033 |
| ZBTB7B | 51043 |
| ZCWPW1 | 55063 |
| ZFPL1 | 7542 |
| ZKSCAN3 | 80317 |
| ZMIZ2 | 83637 |
| ZMYND10 | 51364 |
| ZNF154 | 7710 |
| ZNF205 | 7755 |
| ZNF221 | 7638 |
| ZNF259P | 442240 |
| ZNF280A | 129025 |
| ZNF287 | 57336 |
| ZNF335 | 63925 |
| ZNF358 | 140467 |
| ZNF407 | 55628 |
| ZNF409 | 22830 |
| ZNF444 | 55311 |
| ZNF467 | 168544 |
| ZNF471 | 57573 |
| ZNF556 | 80032 |
| ZNF592 | 9640 |
| ZNF609 | 23060 |
| ZNF646 | 9726 |
| ZNF688 | 146542 |
| ZNF696 | 79943 |
| ZNF717 | 100131827 |
| ZNF771 | 51333 |
| ZNF787 | 126208 |
| ZNF79 | 7633 |
| ZNF8 | 7554 |
| ZNF835 | 90485 |
| ZNRF4 | 148066 |
| ZRSR1 | 7310 |
| ZSWIM1 | 90204 |
| ZZEF1 | 23140 |

TC 11

| | |
|---|---|
| ACTN2 | 88 |
| AKAP6 | 9472 |
| C21ORF62 | 56245 |
| C3ORF51 | 711 |
| CCDC48 | 79825 |
| CCL16 | 6360 |
| CD84 | 8832 |
| CHRNA3 | 1136 |
| CLCNKA | 1187 |
| CPN1 | 1369 |
| CTNNA1 | 1495 |
| DLGAP1 | 9229 |
| DLX2 | 1746 |
| DNAI1 | 27019 |
| DTNA | 1837 |
| EDA | 1896 |
| FLJ11292 | 55338 |
| FLJ12986 | 197319 |
| FLJ14126 | 79907 |
| GABRA5 | 2558 |
| GAS8 | 2622 |
| GPLD1 | 2822 |
| HYAL4 | 23553 |
| JRK | 8629 |
| KIF1A | 547 |
| LHX2 | 9355 |
| LOC92973 | 92973 |
| MAP1A | 4130 |
| MCF2 | 4168 |
| MIER2 | 54531 |
| MPP2 | 4355 |
| MYT1 | 4661 |
| NHLH1 | 4807 |
| NOS1AP | 9722 |
| NPFF | 8620 |
| PAK7 | 57144 |
| PCDH11X | 27328 |
| PKNOX2 | 63876 |
| PLA2G6 | 8398 |
| PRINS | 100169750 |
| RIMS2 | 9699 |
| RPRM | 56475 |
| SBNO1 | 55206 |
| SEZ6L | 23544 |
| SIRT4 | 23409 |
| SLC4A3 | 6508 |
| STK38 | 11329 |
| TMEM151B | 441151 |
| TMEM50A | 23585 |
| TRA@ | 6955 |
| TTLL5 | 23093 |
| UBOX5 | 22888 |
| ZFR2 | 23217 |
| ZNF669 | 79862 |
| ZNF821 | 55565 |

TC 12

| | |
|---|---|
| ABTB2 | 25841 |
| AHDC1 | 27245 |
| BCL2L14 | 79370 |
| BRWD2 | 55717 |
| C18ORF25 | 147339 |
| C2ORF55 | 343990 |
| CHD2 | 1106 |
| CLN6 | 54982 |
| CYTH3 | 9265 |
| DLL3 | 10683 |
| DNAJC4 | 3338 |
| EGLN2 | 112398 |
| FBXO3 | 26273 |
| FOXD3 | 27022 |
| FRMD8 | 83786 |
| GATAD2A | 54815 |
| HECA | 51696 |
| HP1BP3 | 50809 |
| ISYNA1 | 51477 |
| JMJD1C | 221037 |
| KDSR | 2531 |
| KIAA0907 | 22889 |
| LRIG2 | 9860 |
| LRP3 | 4037 |
| LTBR | 4055 |
| MAPK8 | 5599 |
| MLL2 | 8085 |
| MSL1 | 339287 |
| NPC1L1 | 29881 |
| NSL1 | 25936 |
| NTN1 | 9423 |
| OBP2B | 29989 |
| PAPOLG | 64895 |
| PBRM1 | 55193 |
| PHF20L1 | 51105 |
| PIGG | 54872 |
| RBM26 | 64062 |
| RNF126P1 | 376412 |
| SAPS3 | 55291 |
| SDCCAG3 | 10807 |
| SEMA6B | 10501 |
| SLC12A9 | 56996 |
| SLC38A10 | 124565 |
| TMEM132A | 54972 |
| TMEM30B | 161291 |
| TMF1 | 7110 |
| TRAPPC2L | 51693 |
| UBIAD1 | 29914 |
| UBR4 | 23352 |
| USP32 | 84669 |
| VWA1 | 64856 |
| WDR33 | 55339 |
| ZBTB44 | 29068 |
| ZNF654 | 55279 |
| ZNHIT2 | 741 |

TC 13

| | |
|---|---|
| ABI2 | 10152 |
| ALDH3B1 | 221 |
| AP3M2 | 10947 |
| APRT | 353 |
| ARMCX1 | 51309 |
| ARMCX2 | 9823 |
| BEX4 | 56271 |
| C5ORF13 | 9315 |
| C5ORF54 | 63920 |
| CCRL2 | 9034 |
| CEP290 | 80184 |
| CHN1 | 1123 |
| CIRBP | 1153 |
| CSRNP2 | 81566 |
| DPY19L2P2 | 349152 |
| DYNC2LI1 | 51626 |
| DZIP1 | 22873 |
| GDI1 | 2664 |
| GPRASP1 | 9737 |
| GSTA4 | 2941 |
| HDGFRP3 | 50810 |
| HSF2 | 3298 |
| IFT81 | 28981 |
| IFT88 | 8100 |
| IPW | 3653 |
| KIF3A | 11127 |
| LOC65998 | 65998 |
| LRRC37A2 | 474170 |
| LRRC49 | 54839 |
| MAGED2 | 10916 |
| MAGEH1 | 28986 |
| MAGI2 | 9863 |
| MAP9 | 79884 |
| MECP2 | 4204 |
| MEIS2 | 4212 |
| MPST | 4357 |
| MTMR9 | 66036 |
| MYEF2 | 50804 |
| MYH10 | 4628 |
| MYST4 | 23522 |
| MZF1 | 7593 |
| NAP1L3 | 4675 |
| NBEA | 26960 |
| NCRNA00094 | 266655 |
| NCRNA00153 | 55857 |
| NISCH | 11188 |
| PBX1 | 5087 |
| PHC1 | 1911 |
| PHF21A | 51317 |
| POLD4 | 57804 |
| RBM4B | 83759 |
| RHOF | 54509 |
| RUFY3 | 22902 |
| SCAPER | 49855 |
| SDR39U1 | 56948 |
| SETBP1 | 26040 |
| SLC25A12 | 8604 |
| SMARCA1 | 6594 |
| SNRPN | 6638 |
| SSBP2 | 23635 |
| STXBP1 | 6812 |
| SYT11 | 23208 |
| TBC1D19 | 55296 |
| TCF7L1 | 83439 |
| TECPR2 | 9895 |
| TMEFF1 | 8577 |
| TMX4 | 56255 |
| TNFRSF12A | 51330 |
| TRPC1 | 7220 |
| TSC1 | 7248 |
| TUSC3 | 7991 |
| ULK2 | 9706 |
| UNC119B | 84747 |
| USP11 | 8237 |
| WASF1 | 8936 |
| WASF3 | 10810 |
| WDR19 | 57728 |
| WDR7 | 23335 |
| ZCCHC11 | 23318 |
| ZNF10 | 7556 |
| ZNF177 | 7730 |
| ZNF187 | 7741 |
| ZNF271 | 10778 |
| ZNF329 | 79673 |
| ZNF512B | 57473 |
| ZNF516 | 9658 |
| ZNF711 | 7552 |

TC 14

| | |
|---|---|
| ABCA3 | 21 |
| ABHD14A | 25864 |
| ABLIM3 | 22885 |
| ATP6V0A1 | 535 |
| BBS4 | 585 |
| C11ORF60 | 56912 |
| C1ORF114 | 57821 |
| CNDP2 | 55748 |
| CTSF | 8722 |
| DZIP3 | 9666 |
| FAM117A | 81558 |
| FBXL2 | 25827 |
| FLJ22167 | 79583 |
| GABARAP | 11337 |
| GLRB | 2743 |
| HABP4 | 22927 |
| HDAC5 | 10014 |
| HHAT | 55733 |
| IGF2BP2 | 10644 |
| IL8 | 3576 |
| KCTD2 | 23510 |
| LMAN2L | 81562 |
| LRPAP1 | 4043 |
| MARK4 | 57787 |
| NADK | 65220 |
| NAP1L2 | 4674 |
| NFE2L1 | 4779 |
| NGFRAP1 | 27018 |
| NLGN1 | 22871 |
| NME3 | 4832 |
| NME5 | 8382 |
| ORAI3 | 93129 |
| PBXIP1 | 57326 |
| PCDHA9 | 9752 |
| PHF17 | 79960 |
| PIP5K1C | 23396 |
| PLD3 | 23646 |
| PRAF2 | 11230 |
| PSME2 | 5721 |
| RAB11FIP5 | 26056 |
| RAB36 | 9609 |
| RIC8B | 55188 |
| ROGDI | 79641 |
| SAP18 | 10284 |
| SERPINI1 | 5274 |
| SGSH | 6448 |
| SIL1 | 64374 |
| SUOX | 6821 |
| TBC1D17 | 79735 |
| TBC1D9B | 23061 |
| TCTN1 | 79600 |
| TPCN1 | 53373 |
| TUBG2 | 27175 |
| UBXN6 | 80700 |
| VPS11 | 55823 |
| VPS39 | 23339 |

TC 15

| | |
|---|---|
| ALPK1 | 80216 |
| ATF7IP | 55729 |
| ATP8B1 | 5205 |
| C20ORF117 | 140710 |
| C7ORF28B | 221960 |
| C7ORF54 | 27099 |
| DDEF1IT1 | 29065 |
| DIP2A | 23181 |
| FBXW12 | 285231 |
| FKSG49 | 400949 |
| FLJ12151 | 80047 |
| FLJ21272 | 80100 |
| GPR1 | 2825 |
| GTF2H3 | 2967 |
| HCG_1730474 | 643376 |
| KIAA0754 | 643314 |
| KIAA0894 | 22833 |
| LOC152719 | 152719 |
| LOC441258 | 441258 |
| LOC647070 | 647070 |
| LOC653188 | 653188 |
| LOC791120 | 791120 |
| MFSD11 | 79157 |
| NPIPL3 | 23117 |
| NSUN6 | 221078 |
| PCDHGA8 | 9708 |
| PDCD6 | 10016 |
| PODNL1 | 79883 |
| PRR11 | 55771 |
| RP5- | 27308 |
| 886K2.1 | |
| SFRS8 | 6433 |
| SH2B2 | 10603 |
| SPG21 | 51324 |
| SUZ12P | 440423 |
| TAOK1 | 57551 |
| TIGD1L | 414771 |
| TRA2A | 29896 |
| UBQLN4 | 56893 |
| XRCC2 | 7516 |
| ZNF611 | 81856 |
| ZNF701 | 55762 |

TC 16

| | |
|---|---|
| ALMS1 | 7840 |
| AQR | 9716 |
| ASXL1 | 171023 |
| BCL9 | 607 |
| C19ORF10 | 56005 |
| C2CD3 | 26005 |
| C5ORF42 | 65250 |
| CBFA2T2 | 9139 |
| CG012 | 116829 |
| CYB561D2 | 11068 |
| DGCR8 | 54487 |
| DKFZP586I1420 | 222161 |
| FBXO42 | 54455 |
| FLJ10404 | 54540 |
| FLJ13197 | 79667 |
| GLMN | 11146 |
| GON4L | 54856 |
| GTF3C1 | 2975 |
| HMOX2 | 3163 |
| HYMAI | 57061 |
| INPP5E | 56623 |
| INPPL1 | 3636 |
| INTS3 | 65123 |
| KIAA0753 | 9851 |
| KIAA1009 | 22832 |
| LMBR1L | 55716 |
| LOC100134401 | 100134401 |
| LOC100170939 | 100170939 |
| LOC339047 | 339047 |
| LOC399491 | 399491 |
| LRRC37A | 9884 |
| LUC7L | 55692 |
| MADD | 8567 |
| MSH3 | 4437 |
| MTMR15 | 22909 |
| MUM1 | 84939 |
| NAT11 | 79829 |
| NINL | 22981 |
| NOTCH2NL | 388677 |
| NPIP | 9284 |
| PAN2 | 9924 |
| PARP6 | 56965 |
| PILRB | 29990 |
| PLCG1 | 5335 |
| POGZ | 23126 |
| RAB11FIP3 | 9727 |
| RGL2 | 5863 |
| SETD1B | 23067 |
| SFRS14 | 10147 |
| SIN3B | 23309 |
| SLC35E2 | 9906 |
| SMA4 | 11039 |
| SMARCC2 | 6601 |
| SNRNP70 | 6625 |
| TAF9B | 51616 |
| TBC1D3F | 84218 |
| USP20 | 10868 |
| WDR6 | 11180 |
| ZMYM3 | 9203 |
| ZNF133 | 7692 |
| ZNF136 | 7695 |
| ZNF14 | 7561 |
| ZNF211 | 10520 |
| ZNF236 | 7776 |
| ZNF26 | 7574 |
| ZNF273 | 10793 |
| ZNF324 | 25799 |
| ZNF337 | 26152 |
| ZNF43 | 7594 |
| ZNF573 | 126231 |
| ZNF665 | 79788 |
| ZNF692 | 55657 |
| ZNF767 | 79970 |
| ZNF862 | 643641 |
| ZRSR2 | 8233 |

TC 17

| | |
|---|---|
| ARGLU1 | 55082 |
| ARID1A | 8289 |
| ATAD2B | 54454 |
| C11ORF61 | 79684 |
| C21ORF66 | 94104 |
| C2ORF68 | 388969 |
| C4ORF8 | 8603 |
| C9ORF97 | 158427 |
| CDC2L5 | 8621 |
| CHD9 | 80205 |
| CLK4 | 57396 |
| CPSF7 | 79869 |
| CROCCL1 | 84809 |
| CROP | 51747 |
| CSAD | 51380 |
| DDX42 | 11325 |
| DMTF1 | 9988 |
| EFHC1 | 114327 |
| EPM2AIP1 | 9852 |
| FAM48A | 55578 |
| FLJ40113 | 374650 |
| FLJBP1 | 8880 |
| HELZ | 9931 |
| KIAA0240 | 23506 |
| KIAA1704 | 55425 |
| KLHDC10 | 23008 |
| KPNA5 | 3841 |
| LOC220594 | 220594 |
| MAP3K4 | 4216 |
| MON2 | 23041 |
| MYST3 | 7994 |
| N4BP2L2 | 10443 |
| NARG1L | 79612 |
| NBPF10 | 100132406 |
| NBPF14 | 25832 |
| NHLRC2 | 374354 |
| PCM1 | 5108 |
| PDS5B | 23047 |
| PIAS1 | 8554 |
| PMS1 | 5378 |
| PSPC1 | 55269 |
| PTBP2 | 58155 |
| RBM5 | 10181 |
| RBM6 | 10180 |
| REV3L | 5980 |
| RGPD5 | 84220 |
| RSBN1 | 54665 |
| RSRC2 | 65117 |
| S100PBP | 64766 |
| SENP7 | 57337 |
| SFRS11 | 9295 |
| SFRS18 | 25957 |
| SMCHD1 | 23347 |
| SUV420H1 | 51111 |
| TCF12 | 6938 |
| TRIM52 | 84851 |
| TUG1 | 55000 |
| UNC93B1 | 81622 |
| UPF3A | 65110 |
| USP34 | 9736 |
| USP7 | 7874 |
| ZMYM2 | 7750 |
| ZNF207 | 7756 |
| ZNF302 | 55900 |
| ZNF432 | 9668 |
| ZNF451 | 26036 |
| ZNF518A | 9849 |
| ZNF532 | 55205 |
| ZNF638 | 27332 |
| ZNF673 | 55634 |
| ZNF84 | 7637 |

TC 18

| | |
|---|---|
| BAT1 | 7919 |
| BRD3 | 8019 |
| C1ORF63 | 57035 |
| C4ORF29 | 80167 |
| CAPRIN2 | 65981 |
| CCNL2 | 81669 |
| CHD8 | 57680 |
| CLK2 | 1196 |
| CP110 | 9738 |
| DENND4B | 9909 |
| ENOSF1 | 55556 |
| FAM53C | 51307 |
| FTSJD2 | 23070 |
| GOLGA8G | 283768 |
| JARID2 | 3720 |
| LOC440434 | 440434 |
| LRCH3 | 84859 |
| MARK3 | 4140 |
| METTL3 | 56339 |
| MSL2 | 55167 |
| MTA1 | 9112 |
| NFATC2IP | 84901 |
| NPIPL1 | 440350 |
| OFD1 | 8481 |
| PABPN1 | 8106 |
| PCNT | 5116 |
| PHIP | 55023 |
| PI4KA | 5297 |
| POLS | 11044 |
| POU2F1 | 5451 |
| R3HDM2 | 22864 |
| RABGAP1 | 23637 |
| RABL2B | 11158 |
| RBM10 | 8241 |
| TARBP1 | 6894 |
| TAS2R14 | 50840 |
| THOC1 | 9984 |
| TRAPPC10 | 7109 |
| TRIM33 | 51592 |
| USP24 | 23358 |
| ZC3H11A | 9877 |
| ZFYVE26 | 23503 |
| ZNF137 | 7696 |
| ZNF23 | 7571 |
| ZNF266 | 10781 |
| ZNF292 | 23036 |
| ZNF587 | 84914 |
| ZNF652 | 22834 |

TC 19

| | |
|---|---|
| ACIN1 | 22985 |
| ANKZF1 | 55139 |
| ARFGAP1 | 55738 |
| ATG4B | 23192 |
| C1ORF66 | 51093 |
| CDK5RAP3 | 80279 |
| CPSF1 | 29894 |
| E4F1 | 1877 |
| EDC4 | 23644 |
| ENGASE | 64772 |
| FLJ10213 | 55096 |
| GGA1 | 26088 |
| GMEB2 | 26205 |
| KAT2A | 2648 |
| KCTD13 | 253980 |
| KIAA0182 | 23199 |
| KIAA0556 | 23247 |
| MSH5 | 4439 |
| NSUN5 | 55695 |
| NSUN5B | 155400 |
| NSUN5C | 260294 |
| PDXDC2 | 283970 |
| PMS2L2 | 5380 |
| PRR14 | 78994 |
| RAD9A | 5883 |
| RHOT2 | 89941 |
| SFRS16 | 11129 |
| STAG3L1 | 54441 |
| TAF1C | 9013 |
| URG4 | 55665 |
| VPS33B | 26276 |

TC 20

| | |
|---|---|
| ABHD10 | 55347 |
| AKTIP | 64400 |
| ANAPC13 | 25847 |
| ARL3 | 403 |
| ATP5A1 | 498 |
| ATP6V1D | 51382 |
| ATP6V1H | 51606 |
| AUH | 549 |
| BET1 | 10282 |
| C15ORF24 | 56851 |
| C18ORF10 | 25941 |
| C19ORF42 | 79086 |
| C21ORF96 | 80215 |
| CCDC53 | 51019 |
| CGRRF1 | 10668 |
| COPS7A | 50813 |
| COX11 | 1353 |
| COX16 | 51241 |
| DCTN6 | 10671 |
| EBAG9 | 9166 |
| FBXW11 | 23291 |
| FXC1 | 26515 |
| GABARAPL2 | 11345 |
| GIN1 | 54826 |
| GYG1 | 2992 |
| HADHB | 3032 |
| HDDC2 | 51020 |
| HIBCH | 26275 |
| HIGD1A | 25994 |
| IDH3A | 3419 |
| KBTBD4 | 55709 |
| LIPT1 | 51601 |
| LOC100129361 | 100129361 |
| MED7 | 9443 |
| MOCS2 | 4338 |
| MRPL35 | 51318 |
| NDUFAF1 | 51103 |
| NDUFB1 | 4707 |
| NUDT6 | 11162 |
| PDHB | 5162 |
| PGRMC2 | 10424 |
| PIGB | 9488 |
| PIGP | 51227 |
| PPID | 5481 |
| RAD50 | 10111 |
| RWDD1 | 51389 |
| SEC22B | 9554 |
| SEC23B | 10483 |
| SEMA4A | 64218 |
| SERF1A | 8293 |
| SNAPC5 | 10302 |
| SRI | 6717 |
| SRP14 | 6727 |
| TBCA | 6902 |
| THAP1 | 55145 |
| THYN1 | 29087 |
| TRAPPC4 | 51399 |
| TTC19 | 54902 |
| UFSP2 | 55325 |
| UHRF1BP1L | 23074 |

TC 21

| | |
|---|---|
| ACE | 1636 |
| ACTR3B | 57180 |
| AGPAT5 | 55326 |
| AGTPBP1 | 23287 |
| ALKBH1 | 8846 |
| APOOL | 139322 |
| ATP5S | 27109 |
| ATP5SL | 55101 |
| ATXN10 | 25814 |
| C10ORF88 | 80007 |
| C14ORF169 | 79697 |
| CCDC72 | 51372 |
| CPZ | 8532 |
| CUL2 | 8453 |
| DLEU1 | 10301 |
| EIF2AK1 | 27102 |
| ELP4 | 26610 |
| EML3 | 256364 |
| ERCC8 | 1161 |
| EXD2 | 55218 |
| FANCF | 2188 |
| FN3KRP | 79672 |
| FSTL3 | 10272 |
| GPR125 | 166647 |
| GSDMD | 79792 |
| GUF1 | 60558 |
| IKBKAP | 8518 |
| MAK10 | 60560 |
| MYST2 | 11143 |
| NCOR1 | 9611 |
| NFS1 | 9054 |
| NR1H2 | 7376 |
| NSBP1 | 79366 |
| NUPL2 | 11097 |
| OCRL | 4952 |
| PEX1 | 5189 |
| PHF14 | 9678 |
| PHLPPL | 23035 |
| PLK3 | 1263 |
| POLR3F | 10621 |
| PSMD11 | 5717 |
| SBNO2 | 22904 |
| SFXN1 | 94081 |
| SLC24A6 | 80024 |
| SLC39A8 | 64116 |
| SMUG1 | 23583 |
| TBC1D22A | 25771 |
| TCN2 | 6948 |
| THAP10 | 56906 |
| TIMM9 | 26520 |
| TMEM184C | 55751 |
| TMEM5 | 10329 |
| TSGA14 | 95681 |
| TTC30A | 92104 |
| TYW1 | 55253 |
| UNC84B | 25777 |
| USP46 | 64854 |
| WIPI2 | 26100 |
| YEATS4 | 8089 |
| YIPF6 | 286451 |
| ZKSCAN5 | 23660 |
| ZNF180 | 7733 |
| ZNF571 | 51276 |

TC 22

| | |
|---|---|
| ACVR2A | 92 |
| ADAM8 | 101 |
| ADAP1 | 11033 |
| ALG9 | 79796 |
| AMZ2 | 51321 |
| ANAPC10 | 10393 |
| ANKMY2 | 57037 |
| APC | 324 |
| ARL1 | 400 |
| ARMCX3 | 51566 |
| BBS10 | 79738 |
| BBS7 | 55212 |
| BMPR1A | 657 |
| BTBD3 | 22903 |
| C10ORF97 | 80013 |
| C1ORF25 | 81627 |
| C2ORF56 | 55471 |
| C4ORF27 | 54969 |
| C5ORF44 | 80006 |
| CAPN7 | 23473 |
| CBR4 | 84869 |
| CCDC91 | 55297 |
| CDIPT | 10423 |
| CETN2 | 1069 |
| CRBN | 51185 |
| DDHD2 | 23259 |
| DDX24 | 57062 |
| DHX40 | 79665 |
| EID1 | 23741 |
| EXTL2 | 2135 |
| FAM134A | 79137 |
| FAM13B | 51306 |
| FAM172A | 83989 |
| FAM8A1 | 51439 |
| GLT8D1 | 55830 |
| GTF2I | 2969 |
| ISCU | 23479 |
| KCMF1 | 56888 |
| LZTFL1 | 54585 |
| MAP2K4 | 6416 |
| MLH1 | 4292 |
| MOAP1 | 64112 |
| NARG2 | 79664 |
| NDFIP1 | 80762 |
| PCYOX1 | 51449 |
| PNMA1 | 9240 |
| POLI | 11201 |
| PPWD1 | 23398 |
| PREPL | 9581 |
| PRMT2 | 3275 |
| PSIP1 | 11168 |
| PSMC2 | 5701 |
| RANBP6 | 26953 |
| RCBTB1 | 55213 |
| RIOK2 | 55781 |
| RNF146 | 81847 |
| SEC63 | 11231 |
| SECISBP2L | 9728 |
| SFRS12IP1 | 285672 |
| SHB | 6461 |
| SKP1 | 6500 |
| SLC39A6 | 25800 |
| SYNJ1 | 8867 |
| TCEAL1 | 9338 |
| TCEAL4 | 79921 |
| TERF2IP | 54386 |
| TM2D3 | 80213 |
| TMEM92 | 162461 |
| TSPYL1 | 7259 |
| TWSG1 | 57045 |
| USP47 | 55031 |
| WRB | 7485 |
| ZC3H14 | 79882 |
| ZC3H7A | 29066 |
| ZMYND11 | 10771 |
| ZNF226 | 7769 |
| ZNF280D | 54816 |
| ZNF45 | 7596 |

TC 23

| | |
|---|---|
| ABCD1 | 215 |
| ACVR1 | 90 |
| ANXA7 | 310 |
| ATP6AP2 | 10159 |
| BICD2 | 23299 |
| BNIP2 | 663 |
| BTNL3 | 10917 |
| CBFB | 865 |
| CCDC82 | 79780 |
| CDX2 | 1045 |
| CEP170 | 9859 |
| CGGBP1 | 8545 |
| CHSY1 | 22856 |
| CLDND1 | 56650 |
| CRYZL1 | 9946 |
| CSGALNACT2 | 55454 |
| CSNK1A1 | 1452 |
| DHX34 | 9704 |
| EFR3A | 23167 |
| ELOVL5 | 60481 |
| EPS15 | 2060 |
| GOLGA7 | 51125 |
| GPATCH4 | 54865 |
| HNF1A | 6927 |
| HNF4A | 3172 |
| HR | 55806 |
| INPP4A | 3631 |
| ITPK1 | 3705 |
| KAZALD1 | 81621 |
| KIAA0430 | 9665 |
| MAP3K7IP2 | 23118 |
| MAP4K5 | 11183 |
| MARK2 | 2011 |
| MFAP3 | 4238 |
| MTMR6 | 9107 |
| MTR | 4548 |
| MUC3A | 4584 |
| NCDN | 23154 |
| NEK7 | 140609 |
| NFYB | 4801 |
| NPTN | 27020 |
| OSBPL8 | 114882 |
| PAFAH1B1 | 5048 |
| PPP1R12A | 4659 |
| PRKD3 | 23683 |
| PRRG2 | 5639 |
| RAB21 | 23011 |
| RBPJ | 3516 |
| RECQL | 5965 |
| SEC23A | 10484 |
| SEPT10 | 151011 |
| SEPT7 | 989 |
| SLC19A1 | 6573 |
| SOCS5 | 9655 |
| SPAG9 | 9043 |
| SPG20 | 23111 |
| SPRED2 | 200734 |
| TBC1D2B | 23102 |
| TMED7 | 51014 |
| TNK1 | 8711 |
| TOR1AIP1 | 26092 |
| USP25 | 29761 |
| WAC | 51322 |
| WBP5 | 51186 |
| WDR26 | 80232 |
| WDR82 | 80335 |
| YPEL5 | 51646 |

TC 24

| | |
|---|---|
| ABCD3 | 5825 |
| ACAN | 176 |
| ACAP2 | 23527 |
| ACSL3 | 2181 |
| ADO | 84890 |
| ADSS | 159 |
| AGGF1 | 55109 |
| AGL | 178 |
| AKAP11 | 11215 |
| ALG13 | 79868 |
| ALG6 | 29929 |
| ANGEL2 | 90806 |
| ANKRA2 | 57763 |
| ANKRD17 | 26057 |
| ANKRD27 | 84079 |
| ARHGAP5 | 394 |
| ARID4A | 5926 |
| ARL5A | 26225 |
| ARMC1 | 55156 |
| ARMCX5 | 64860 |
| ARPP19 | 10776 |
| ATMIN | 23300 |
| ATP11B | 23200 |
| ATP2C1 | 27032 |
| ATR | 545 |
| ATRX | 546 |
| BAZ1B | 9031 |
| BAZ2B | 29994 |
| BMI1 | 648 |
| BTAF1 | 9044 |
| BTBD1 | 53339 |
| C10ORF18 | 54906 |
| C12ORF29 | 91298 |
| C14ORF104 | 55172 |
| C1ORF109 | 54955 |
| C1ORF149 | 64769 |
| C1ORF174 | 339448 |
| C4ORF30 | 54876 |
| C5ORF22 | 55322 |
| C9ORF82 | 79886 |
| CCDC90B | 60492 |
| CCL22 | 6367 |
| CCNT2 | 905 |
| CD22 | 933 |
| CD300C | 10871 |
| CD5 | 921 |
| CDC23 | 8697 |
| CDC27 | 996 |
| CDC73 | 79577 |
| CDKN1B | 1027 |
| CDKN2AIP | 55602 |
| CETN3 | 1070 |
| CHD1 | 1105 |
| CHERP | 10523 |
| CHRD | 8646 |
| CHUK | 1147 |
| CLPX | 10845 |
| CNOT4 | 4850 |
| CNOT6 | 57472 |
| COMMD8 | 54951 |
| COPB1 | 1315 |
| CRY1 | 1407 |
| CSNK1G3 | 1456 |
| CTR9 | 9646 |
| DCK | 1633 |
| DDX46 | 9879 |
| DDX5 | 1655 |
| DHX29 | 54505 |
| DNAJB5 | 25822 |
| DNAJC24 | 120526 |
| DPY19L4 | 286148 |
| DYRK1A | 1859 |
| EBI3 | 10148 |
| EFHA1 | 221154 |
| EGO | 100126791 |
| EIF1AX | 1964 |
| EIF3A | 8661 |
| EIF4G2 | 1982 |
| ELL | 8178 |
| ENOPH1 | 58478 |
| ERBB2IP | 55914 |
| ETNK1 | 55500 |
| FAM179B | 23116 |
| FAM18B | 51030 |
| FASTKD3 | 79072 |
| FBXO11 | 80204 |
| FBXO38 | 81545 |
| FKBP8 | 23770 |
| FMR1 | 2332 |
| FNBP1L | 54874 |
| FUBP3 | 8939 |
| GBAS | 2631 |
| GNG10 | 2790 |
| GOLPH3 | 64083 |
| GRSF1 | 2926 |
| GTF2H1 | 2965 |
| H2AFV | 94239 |
| HISPPD1 | 23262 |
| HLA-DOA | 3111 |
| HMG20A | 10363 |
| HNRNPA2B1 | 3181 |
| HNRNPA3 | 220988 |
| HNRPDL | 9987 |
| HS2ST1 | 9653 |
| HSPA13 | 6782 |
| HSPB11 | 51668 |
| IBTK | 25998 |
| ICOSLG | 23308 |
| IER3IP1 | 51124 |
| IL3RA | 3563 |
| IMPA1 | 3612 |
| IPO7 | 10527 |
| ISOC1 | 51015 |
| KCNAB2 | 8514 |
| KDM3B | 51780 |
| KIAA0232 | 9778 |
| KIAA0317 | 9870 |
| KIAA0368 | 23392 |
| KIAA0892 | 23383 |
| KIAA0947 | 23379 |
| KIAA1012 | 22878 |
| KIFC3 | 3801 |
| KRIT1 | 889 |
| KTN1 | 3895 |
| LARS | 51520 |
| LDB1 | 8861 |
| LEMD3 | 23592 |
| LILRA2 | 11027 |
| LILRB3 | 11025 |
| LRBA | 987 |
| LRRC47 | 57470 |
| LUC7L2 | 51631 |
| LYL1 | 4066 |
| MAEA | 10296 |
| MAML1 | 9794 |
| MAP4K3 | 8491 |
| MAPK1IP1L | 93487 |
| MAPKSP1 | 8649 |
| MARCH7 | 64844 |
| MATR3 | 9782 |
| MED23 | 9439 |
| MED4 | 29079 |
| MINPP1 | 9562 |
| MIS12 | 79003 |
| MORC3 | 23515 |
| MPRIP | 23164 |
| MRFAP1L1 | 114932 |
| MRS2 | 57380 |
| MTMR1 | 8776 |
| MTX2 | 10651 |
| MUDENG | 55745 |
| NARS | 4677 |
| NDUFA5 | 4698 |
| NECAP1 | 25977 |
| NEIL1 | 79661 |
| NEK4 | 6787 |
| NFIC | 4782 |
| NUP153 | 9972 |
| OPA1 | 4976 |
| PAQR3 | 152559 |
| PDCL3 | 79031 |
| PDE12 | 201626 |
| PDGFB | 5155 |
| PDHX | 8050 |
| PDS5A | 23244 |
| PIGK | 10026 |
| PIKFYVE | 200576 |
| PLD2 | 5338 |
| PLEKHA4 | 57664 |
| PLEKHH3 | 79990 |
| PMPCB | 9512 |
| POT1 | 25913 |
| POU5F1B | 5462 |
| PPM1B | 5495 |
| PPP1R8 | 5511 |
| PPP2R5C | 5527 |
| PPP3CB | 5532 |
| PPP4R2 | 151987 |
| PPP6C | 5537 |
| PRPF39 | 55015 |
| PRPF4B | 8899 |
| PRRX2 | 51450 |
| PTPLB | 201562 |
| PUM1 | 9698 |
| PUM2 | 23369 |
| QTRTD1 | 79691 |
| RAB28 | 9364 |
| RANBP2 | 5903 |
| RAP2C | 57826 |
| RASGRP2 | 10235 |
| RB1CC1 | 9821 |
| RBM16 | 22828 |
| RBM25 | 58517 |
| RCHY1 | 25898 |
| RDH14 | 57665 |
| RETN | 56729 |
| REV1 | 51455 |
| RHOT1 | 55288 |
| RNF11 | 26994 |
| RNF111 | 54778 |
| RNF139 | 11236 |
| RNF38 | 152006 |
| RNF4 | 6047 |
| RNF6 | 6049 |
| RNPEPL1 | 57140 |
| RPA2 | 6118 |
| RRN3 | 54700 |
| RUNX1 | 861 |
| RWDD3 | 25950 |
| S1PR4 | 8698 |
| SACM1L | 22908 |
| SCFD1 | 23256 |
| SCYL2 | 55681 |
| SDCCAG1 | 9147 |
| SEC16A | 9919 |
| SEC24B | 10427 |
| SETD2 | 29072 |
| SFRS12 | 140890 |
| SGCA | 6442 |
| SIGLEC7 | 27036 |
| SIRT1 | 23411 |
| SIT1 | 27240 |
| SLC11A1 | 6556 |
| SLC25A46 | 91137 |
| SLC2A3P1 | 100128062 |
| SLC30A9 | 10463 |
| SLC6A7 | 6534 |
| SLTM | 79811 |
| SMAD2 | 4087 |
| SMAD4 | 4089 |
| SMAD5 | 4090 |
| SMAP1 | 60682 |
| SMARCA5 | 8467 |
| SMNDC1 | 10285 |
| SON | 6651 |
| SQSTM1 | 8878 |
| SR140 | 23350 |
| STAM | 8027 |
| STAM2 | 10254 |
| STAU1 | 6780 |
| STRN3 | 29966 |
| SUCLA2 | 8803 |
| TAF7 | 6879 |
| TIA1 | 7072 |
| TM6SF2 | 53345 |
| TMEM131 | 23505 |
| TMEM165 | 55858 |
| TMEM33 | 55161 |
| TMEM41B | 440026 |
| TOP2B | 7155 |
| TRAPPC2 | 6399 |
| TRIM37 | 4591 |
| TRMT61B | 55006 |
| TSNAX | 7257 |
| TSPAN32 | 10077 |
| TSPYL4 | 23270 |
| TTC37 | 9652 |
| TXNL1 | 9352 |
| UBA3 | 9039 |
| UBE2I | 7329 |
| UBE2K | 3093 |
| UBE3C | 9690 |
| UBE4A | 9354 |
| UBP1 | 7342 |
| UBQLN2 | 29978 |
| UBR5 | 51366 |
| UBR7 | 55148 |
| USP14 | 9097 |
| USP33 | 23032 |
| USP48 | 84196 |
| USP8 | 9101 |
| VEZF1 | 7716 |
| VEZT | 55591 |
| VPS4B | 9525 |
| VPS54 | 51542 |
| WDR47 | 22911 |
| WSB2 | 55884 |
| YTHDC2 | 64848 |
| YTHDF3 | 253943 |
| YY1 | 7528 |
| ZBTB11 | 27107 |
| ZC3H13 | 23091 |
| ZC3H4 | 23211 |
| ZCCHC10 | 54819 |
| ZCCHC14 | 23174 |
| ZCCHC8 | 55596 |
| ZFYVE16 | 9765 |
| ZMIZ1 | 57178 |
| ZMYM4 | 9202 |
| ZNF362 | 149076 |
| ZNF410 | 57862 |
| ZNF529 | 57711 |
| ZNHIT6 | 54680 |
| ZZZ3 | 26009 |

TC 25

| | |
|---|---|
| AKAP13 | 11214 |
| ANKRD36B | 57730 |
| BAT2D1 | 23215 |
| BBX | 56987 |
| BRD2 | 6046 |
| CBX5 | 23468 |
| COIL | 8161 |
| COL4A3BP | 10087 |
| DNAJB14 | 79982 |
| DNAJC3 | 5611 |
| EIF5B | 9669 |
| EPRS | 2058 |
| ESF1 | 51575 |
| FAF2 | 23197 |
| FUS | 2521 |
| GLG1 | 2734 |
| HIPK1 | 204851 |
| IGF2R | 3482 |
| LEPROT | 54741 |
| MED1 | 5469 |
| MORF4L2 | 9643 |
| NFAT5 | 10725 |
| NKTR | 4820 |
| NUCKS1 | 64710 |
| PKN2 | 5586 |
| PPFIBP1 | 8496 |
| PPIG | 9360 |
| RASA2 | 5922 |
| RYBP | 23429 |
| SECISBP2 | 79048 |
| SF3B1 | 23451 |
| SNX27 | 81609 |
| SPEN | 23013 |
| SRRM1 | 10250 |
| TAF15 | 8148 |
| TNPO1 | 3842 |
| TNPO3 | 23534 |
| TNRC6B | 23112 |
| TTF1 | 7270 |
| TULP4 | 56995 |
| UBXN7 | 26043 |
| VGLL4 | 9686 |
| WNK1 | 65125 |
| ZBTB43 | 23099 |
| ZNF124 | 7678 |
| ZNF148 | 7707 |
| ZNF24 | 7572 |
| ZNF562 | 54811 |

TC 26

| | |
|---|---|
| ABCF1 | 23 |
| ACAT2 | 39 |
| ACN9 | 57001 |
| ALAS1 | 211 |
| ALG8 | 79053 |
| AMD1 | 262 |
| AMMECR1 | 9949 |
| ANAPC1 | 64682 |
| ANP32A | 8125 |
| ANP32B | 10541 |
| APEX1 | 328 |
| ARHGAP11A | 9824 |
| ARHGEF15 | 22899 |
| ARL6IP1 | 23204 |
| ARPC5L | 81873 |
| ASCC3 | 10973 |
| ASNS | 440 |
| ASNSD1 | 54529 |
| ATAD2 | 29028 |
| ATF1 | 466 |
| ATF7 | 11016 |
| ATG5 | 9474 |
| ATIC | 471 |
| AZIN1 | 51582 |
| BARD1 | 580 |
| BCAS2 | 10286 |
| BRCA1 | 672 |
| BRCA2 | 675 |
| BRCC3 | 79184 |
| BRD7 | 29117 |
| BTG3 | 10950 |
| BXDC2 | 55299 |
| BYSL | 705 |
| BZW2 | 28969 |
| C11ORF10 | 746 |
| C11ORF58 | 10944 |
| C11ORF73 | 51501 |
| C12ORF48 | 55010 |
| C12ORF5 | 57103 |
| C13ORF23 | 80209 |
| C13ORF27 | 93081 |
| C13ORF34 | 79866 |
| C14ORF109 | 26175 |
| C14ORF166 | 51637 |
| C16ORF61 | 56942 |
| C17ORF75 | 64149 |
| C18ORF24 | 220134 |
| C1D | 10438 |
| C1ORF112 | 55732 |
| C1ORF135 | 79000 |
| C1QBP | 708 |
| C20ORF11 | 54994 |
| C20ORF20 | 55257 |
| C20ORF43 | 51507 |
| C20ORF7 | 79133 |
| C21ORF45 | 54069 |
| C2ORF47 | 79568 |
| C7ORF28A | 51622 |
| CACYBP | 27101 |
| CAMTA1 | 23261 |
| CBWD1 | 55871 |
| CBX7 | 23492 |
| CCDC21 | 64793 |
| CCDC47 | 57003 |
| CCDC59 | 29080 |
| CCDC90A | 63933 |
| CCDC99 | 54908 |
| CCNC | 892 |
| CCNE1 | 898 |
| CCNH | 902 |
| CCT2 | 10576 |
| CCT6A | 908 |
| CCT8 | 10694 |
| CDC123 | 8872 |
| CDC5L | 988 |
| CDC6 | 990 |
| CDC7 | 8317 |
| CDCA4 | 55038 |
| CDT1 | 81620 |
| CEBPZ | 10153 |
| CECR5 | 27440 |
| CENPI | 2491 |
| CENPJ | 55835 |
| CENPM | 79019 |
| CEP55 | 55165 |
| CEP72 | 55722 |
| CHCHD3 | 54927 |
| CHEK2 | 11200 |
| CHMP5 | 51510 |
| CIAPIN1 | 57019 |
| CKAP5 | 9793 |
| CKS1B | 1163 |
| CLNS1A | 1207 |
| CLTA | 1211 |
| CLU | 1191 |
| CNBP | 7555 |
| CNIH | 10175 |
| CNIH4 | 29097 |
| CNOT1 | 23019 |
| COPS2 | 9318 |
| COPS4 | 51138 |
| COPS5 | 10987 |
| COPS8 | 10920 |
| COX4NB | 10328 |
| COX5A | 9377 |
| CRIPT | 9419 |
| CSE1L | 1434 |
| CSNK2A1 | 1457 |
| CSTF1 | 1477 |
| CTPS | 1503 |
| DAP3 | 7818 |
| DBF4 | 10926 |
| DDX1 | 1653 |
| DDX18 | 8886 |
| DDX21 | 9188 |
| DEPDC1 | 55635 |
| DGUOK | 1716 |
| DHFR | 1719 |
| DHX9 | 1660 |
| DIABLO | 56616 |
| DIAPH3 | 81624 |
| DIMT1L | 27292 |
| DKC1 | 1736 |
| DLAT | 1737 |
| DLD | 1738 |
| DLGAP5 | 9787 |
| DNA2 | 1763 |
| DNAJA1 | 3301 |
| DNAJA2 | 10294 |
| DNAJB6 | 10049 |
| DNAJC2 | 27000 |
| DNAJC9 | 23234 |
| DNMT1 | 1786 |
| DNMT3B | 1789 |
| DNTTIP2 | 30836 |
| DPM1 | 8813 |
| DR1 | 1810 |
| DTL | 51514 |
| DYNC1LI1 | 51143 |
| DYNLL1 | 8655 |
| E2F3 | 1871 |
| E2F5 | 1875 |
| E2F8 | 79733 |
| EBF2 | 64641 |
| EEF1E1 | 9521 |
| EIF2B1 | 1967 |
| EIF2S1 | 1965 |
| EIF2S3 | 1968 |
| EIF3J | 8669 |
| EIF3M | 10480 |
| EIF4E | 1977 |
| EIF5 | 1983 |
| EMG1 | 10436 |
| ERCC6L | 54821 |
| ETFA | 2108 |
| EXOC5 | 10640 |
| EXOSC2 | 23404 |
| EXOSC8 | 11340 |
| EZH2 | 2146 |
| FAM136A | 84908 |
| FAM45B | 55855 |
| FANCA | 2175 |
| FANCG | 2189 |
| FBXO22 | 26263 |
| FNTA | 2339 |
| FTSJ1 | 24140 |
| FTSJ2 | 29960 |
| G3BP2 | 9908 |
| GAR1 | 54433 |
| GCN1L1 | 10985 |
| GCSH | 2653 |
| GFM1 | 85476 |
| GGCT | 79017 |
| GGH | 8836 |
| GINS2 | 51659 |
| GINS3 | 64785 |
| GLO1 | 2739 |
| GLOD4 | 51031 |
| GLRX2 | 51022 |
| GLRX3 | 10539 |
| GMFB | 2764 |
| GMNN | 51053 |
| GNL2 | 29889 |
| GNL3 | 26354 |
| GOLT1B | 51026 |
| GORASP2 | 26003 |
| GPN1 | 11321 |
| GPN3 | 51184 |
| GPSM2 | 29899 |
| GTF2A2 | 2958 |
| GTF2E2 | 2961 |
| GTF2H5 | 404672 |
| GTPBP4 | 23560 |
| HAT1 | 8520 |
| HAUS2 | 55142 |
| HCCS | 3052 |
| HDAC1 | 3065 |
| HDAC2 | 3066 |
| HEATR1 | 55127 |
| HELLS | 3070 |
| HMGB1 | 3146 |
| HMGB3L1 | 128872 |
| HMGCR | 3156 |
| HMGN1 | 3150 |
| HN1 | 51155 |
| HNRNPAB | 3182 |
| HPRT1 | 3251 |
| HSP90AA1 | 3320 |
| HSPA14 | 51182 |
| HSPA4 | 3308 |
| HSPA9 | 3313 |
| HSPE1 | 3336 |
| HSPH1 | 10808 |
| IARS | 3376 |
| IARS2 | 55699 |
| IGF2BP3 | 10643 |
| ILF2 | 3608 |
| IMMT | 10989 |
| IMPAD1 | 54928 |
| INTS12 | 57117 |
| INTS8 | 55656 |
| ISCA1 | 81689 |
| ITGAE | 3682 |
| ITGB3BP | 23421 |
| ITIH4 | 3700 |
| KARS | 3735 |
| KDM1 | 23028 |
| KIAA0020 | 9933 |
| KIAA0391 | 9692 |
| KIF15 | 56992 |
| KIF18A | 81930 |
| KIF20B | 9585 |
| KIF23 | 9493 |
| KNTC1 | 9735 |
| KPNA4 | 3840 |
| KPNB1 | 3837 |
| LASS6 | 253782 |
| LBR | 3930 |
| LIG1 | 3978 |
| LIN7C | 55327 |
| LMF2 | 91289 |
| LMNB2 | 84823 |
| LSM1 | 27257 |
| LSM5 | 23658 |
| LSM6 | 11157 |
| LSM8 | 51691 |
| LYPLA1 | 10434 |
| MAGOH | 4116 |
| MAGOHB | 55110 |
| MAP2K1 | 5604 |
| MAPK6 | 5597 |
| MAPKAPK5 | 8550 |
| MARCH5 | 54708 |
| MCM5 | 4174 |
| MCTS1 | 28985 |
| MED21 | 9412 |
| MED28 | 80306 |
| MED6 | 10001 |
| METAP1 | 23173 |
| METAP2 | 10988 |
| METTL13 | 51603 |
| METTL2B | 55798 |
| MFAP1 | 4236 |
| MFF | 56947 |
| MFN1 | 55669 |
| MOBKL3 | 25843 |
| MPHOSPH10 | 10199 |
| MPP5 | 64398 |
| MRPL13 | 28998 |
| MRPL15 | 29088 |
| MRPL3 | 11222 |
| MRPL39 | 54148 |
| MRPL42 | 28977 |
| MRPL9 | 65005 |
| MRPS10 | 55173 |
| MRPS27 | 23107 |
| MRPS30 | 10884 |
| MSH2 | 4436 |
| MSH6 | 2956 |
| MTCH2 | 23788 |
| MTERFD1 | 51001 |
| MTFR1 | 9650 |
| MTHFD2 | 10797 |
| MTIF2 | 4528 |
| MYCBP | 26292 |
| NAT10 | 55226 |
| NCAPD2 | 9918 |
| NCAPD3 | 23310 |
| NCAPG | 64151 |
| NCBP2 | 22916 |
| NCL | 4691 |
| NDC80 | 10403 |
| NEIL3 | 55247 |
| NEK2 | 4751 |
| NFATC4 | 4776 |
| NFU1 | 27247 |
| NGDN | 25983 |
| NIF3L1 | 60491 |
| NIP7 | 51388 |
| NIPA2 | 81614 |
| NOL11 | 25926 |
| NOL7 | 51406 |
| NONO | 4841 |
| NPEPPS | 9520 |
| NPM3 | 10360 |
| NSMCE4A | 54780 |
| NT5DC2 | 64943 |
| NUDT15 | 55270 |
| NUDT21 | 11051 |
| NUP107 | 57122 |
| NUP155 | 9631 |
| NUP205 | 23165 |
| NUP37 | 79023 |
| NUP50 | 10762 |
| NUP62 | 23636 |
| NUP85 | 79902 |
| NUP93 | 9688 |
| NXT1 | 29107 |
| ODC1 | 4953 |
| OLA1 | 29789 |
| ORC2L | 4999 |
| ORC5L | 5001 |
| OXSR1 | 9943 |
| PAFAH1B3 | 5050 |
| PAICS | 10606 |
| PAK1IP1 | 55003 |
| PAPOLA | 10914 |
| PARP1 | 142 |
| PBK | 55872 |
| PCID2 | 55795 |
| PCMT1 | 5110 |
| PCNA | 5111 |
| PDCD10 | 11235 |
| PFDN2 | 5202 |
| PGK1 | 5230 |
| PIGF | 5281 |
| PINK1 | 65018 |
| PLCB2 | 5330 |
| PLK4 | 10733 |
| PNO1 | 56902 |
| POLA2 | 23649 |
| POLB | 5423 |
| POLD1 | 5424 |
| POLD3 | 10714 |
| POLE3 | 54107 |
| POLR1B | 84172 |
| POLR2B | 5431 |
| POLR2D | 5433 |
| POLR2G | 5436 |
| POLR2K | 5440 |
| POMP | 51371 |
| POP5 | 51367 |
| PPAT | 5471 |
| PPIA | 5478 |
| PPP2R3C | 55012 |
| PRICKLE4 | 29964 |
| PRIM1 | 5557 |
| PRIM2 | 5558 |
| PRKDC | 5591 |
| PRKRA | 8575 |
| PRMT1 | 3276 |
| PRMT3 | 10196 |
| PRPF19 | 27339 |
| PRPF4 | 9128 |
| PSAT1 | 29968 |
| PSMA2 | 5683 |
| PSMA4 | 5685 |
| PSMA6 | 5687 |
| PSMB1 | 5689 |
| PSMC3IP | 29893 |
| PSMC6 | 5706 |
| PSMD10 | 5716 |
| PSMD12 | 5718 |
| PSMD14 | 10213 |
| PSMD6 | 9861 |
| PSMG1 | 8624 |
| PSMG2 | 56984 |
| PSRC1 | 84722 |
| PTDSS1 | 9791 |
| PTGES3 | 10728 |
| PTPN11 | 5781 |
| PTS | 5805 |
| PTTG3 | 26255 |
| PUS7 | 54517 |
| RAB11A | 8766 |
| RAB22A | 57403 |
| RAD21 | 5885 |
| RAD23B | 5887 |
| RAD51 | 5888 |
| RAD51AP1 | 10635 |
| RAD51C | 5889 |
| RAD54B | 25788 |
| RAD54L | 8438 |
| RAE1 | 8480 |
| RAN | 5901 |
| RAP1GDS1 | 5910 |
| RAPGEF3 | 10411 |
| RARS2 | 57038 |
| RBL1 | 5933 |
| RFC2 | 5982 |
| RFC3 | 5983 |
| RFC5 | 5985 |
| RFWD3 | 55159 |
| RMI1 | 80010 |
| RNF114 | 55905 |
| RNF7 | 9616 |
| RPE | 6120 |
| RPIA | 22934 |
| RPL26L1 | 51121 |
| RPP30 | 10556 |
| RPP40 | 10799 |
| RRM1 | 6240 |
| RSL24D1 | 51187 |
| SAC3D1 | 29901 |
| SAE1 | 10055 |
| SC4MOL | 6307 |
| SCYE1 | 9255 |
| SEP15 | 9403 |
| SERBP1 | 26135 |
| SET | 6418 |
| SF3A1 | 10291 |
| SF3B3 | 23450 |
| SFRS9 | 8683 |
| SHCBP1 | 79801 |
| SIP1 | 8487 |
| SKIV2L2 | 23517 |
| SKP2 | 6502 |
| SLC25A32 | 81034 |
| SLC4A1AP | 22950 |
| SLMO2 | 51012 |
| SMC2 | 10592 |
| SMC4 | 10051 |
| SMS | 6611 |
| SNRNP27 | 11017 |
| SNRPA | 6626 |
| SNRPA1 | 6627 |
| SNRPB2 | 6629 |
| SNRPD1 | 6632 |
| SNRPE | 6635 |
| SNRPG | 6637 |
| SNW1 | 22938 |
| SPATA5L1 | 79029 |
| SPC25 | 57405 |
| SPTLC1 | 10558 |
| SQLE | 6713 |
| SRP19 | 6728 |
| SRP54 | 6729 |
| SRP72 | 6731 |
| SRP9 | 6726 |
| SRPK1 | 6732 |
| SS18L2 | 51188 |
| SSB | 6741 |
| SSBP1 | 6742 |
| SSRP1 | 6749 |
| STARD7 | 56910 |
| STIL | 6491 |
| STRAP | 11171 |
| SUB1 | 10923 |
| SUMO1 | 7341 |
| TACC3 | 10460 |
| TAF5 | 6877 |
| TARS | 6897 |
| TCEA1 | 6917 |
| TCEB1 | 6921 |
| TCP1 | 6950 |
| TFB2M | 64216 |
| TFEB | 7942 |
| TH1L | 51497 |
| THOC7 | 80145 |
| TIMM17A | 10440 |
| TIMM23 | 10431 |
| TIPIN | 54962 |
| TK1 | 7083 |
| TK2 | 7084 |
| TMCO1 | 54499 |
| TMEM126B | 55863 |
| TMEM14A | 28978 |
| TMEM14B | 81853 |
| TMEM194A | 23306 |
| TMEM48 | 55706 |
| TMEM97 | 27346 |
| TMX2 | 51075 |
| TNFSF12 | 8742 |
| TNXA | 7146 |
| TOMM70A | 9868 |
| TPRKB | 51002 |
| TRAIP | 10293 |
| TRIM28 | 10155 |
| TRIP12 | 9320 |
| TRMT5 | 57570 |
| TSEN34 | 79042 |
| TSN | 7247 |
| TSR1 | 55720 |
| TTC35 | 9694 |
| TTF2 | 8458 |
| TTRAP | 51567 |
| TUBA1B | 10376 |
| TUBA1C | 84790 |
| TUBB | 203068 |
| TUBG1 | 7283 |
| TXNDC9 | 10190 |
| TXNIP | 10628 |
| TYMS | 7298 |
| UBAP2L | 9898 |
| UBE2A | 7319 |
| UBE2D2 | 7322 |
| UBE2E1 | 7324 |
| UBE2E3 | 10477 |
| UBE2G1 | 7326 |
| UBFD1 | 56061 |
| UCHL5 | 51377 |
| UCK2 | 7371 |
| UMPS | 7372 |
| UNG | 7374 |
| USP1 | 7398 |
| USP39 | 10713 |
| UTP11L | 51118 |
| UTP3 | 57050 |
| UTP6 | 55813 |
| UXS1 | 80146 |
| VAMP7 | 6845 |
| VBP1 | 7411 |
| VDAC3 | 7419 |
| VPS26A | 9559 |
| VPS35 | 55737 |
| VPS72 | 6944 |
| VRK1 | 7443 |
| WDHD1 | 11169 |
| WDR3 | 10885 |
| WDR4 | 10785 |
| WDR43 | 23160 |
| WDR45L | 56270 |
| WDR67 | 93594 |
| WDSOF1 | 25879 |
| WDYHV1 | 55093 |
| WHSC1 | 7468 |
| XPOT | 11260 |
| XRCC5 | 7520 |
| YARS2 | 51067 |
| YEATS2 | 55689 |
| YES1 | 7525 |
| YME1L1 | 10730 |
| YRDC | 79693 |
| YTHDF1 | 54915 |
| ZC3H15 | 55854 |
| ZDHHC6 | 64429 |
| ZNF330 | 27309 |
| ZNHIT3 | 9326 |
| ZWILCH | 55055 |

TC 27

| | |
|---|---|
| AATF | 26574 |
| ABCA6 | 23460 |
| ABCF2 | 10061 |
| ABT1 | 29777 |
| ACOT7 | 11332 |
| ACP1 | 52 |
| ADRM1 | 11047 |
| ADSL | 158 |
| AHCY | 191 |
| AHSA1 | 10598 |
| APEX2 | 27301 |
| APOBEC3B | 9582 |
| ARMET | 7873 |
| ATP5J2 | 9551 |
| AUP1 | 550 |
| BANF1 | 8815 |
| BCCIP | 56647 |
| BCS1L | 617 |
| BRMS1 | 25855 |
| BTG2 | 7832 |
| BUD31 | 8896 |
| C11ORF48 | 79081 |
| C12ORF52 | 84934 |
| C14ORF156 | 81892 |
| C14ORF2 | 9556 |
| C9ORF40 | 55071 |
| CARS | 833 |
| CCDC86 | 79080 |
| CCT3 | 7203 |
| CCT4 | 10575 |
| CCT7 | 10574 |
| CDC25B | 994 |
| CDC34 | 997 |
| CDK4 | 1019 |
| CDK5RAP1 | 51654 |
| COPS3 | 8533 |
| COPS6 | 10980 |
| CSNK2B | 1460 |
| CSTF2 | 1478 |
| CYC1 | 1537 |
| DARS2 | 55157 |
| DCPS | 28960 |
| DCTPP1 | 79077 |
| DDX27 | 55661 |
| DDX56 | 54606 |
| DHCR7 | 1717 |
| DNAJA3 | 9093 |
| DSN1 | 79980 |
| DTYMK | 1841 |
| DUS1L | 64118 |
| DUS4L | 11062 |
| EBNA1BP2 | 10969 |
| EBP | 10682 |
| EIF4A1 | 1973 |
| EIF4A3 | 9775 |
| EIF4E2 | 9470 |
| EIF6 | 3692 |
| ELOVL6 | 79071 |
| ERAL1 | 26284 |
| EXOSC4 | 54512 |
| EXOSC5 | 56915 |
| EXOSC9 | 5393 |
| FAM107A | 11170 |
| FAM128A | 653784 |
| FAM158A | 51016 |
| FARSA | 2193 |
| FBL | 2091 |
| FDPS | 2224 |
| FKBP4 | 2288 |
| FLAD1 | 80308 |
| FZD4 | 8322 |
| GABARAPL1 | 23710 |
| GAPDH | 2597 |
| GARS | 2617 |
| GEMIN4 | 50628 |
| GEMIN6 | 79833 |
| GOT2 | 2806 |
| GRPEL1 | 80273 |
| GSS | 2937 |
| IMP4 | 92856 |
| IPO4 | 79711 |
| ITPA | 3704 |
| JTV1 | 7965 |
| LAGE3 | 8270 |
| LARS2 | 23395 |
| LAS1L | 81887 |
| LBA1 | 9881 |
| LOC388796 | 388796 |
| LOC728344 | 728344 |
| LONP1 | 9361 |
| LRP8 | 7804 |
| LSM12 | 124801 |
| LSM2 | 57819 |
| LSM4 | 25804 |
| LSM7 | 51690 |
| MAST4 | 375449 |
| MIF | 4282 |
| MLEC | 9761 |
| MLF2 | 8079 |
| MRPL11 | 65003 |
| MRPL12 | 6182 |
| MRPL17 | 63875 |
| MRPL18 | 29074 |
| MRPL2 | 51069 |
| MRPL23 | 6150 |
| MRPL48 | 51642 |
| MRPS15 | 64960 |
| MRPS16 | 51021 |
| MRPS17 | 51373 |
| MRPS18A | 55168 |
| MRPS2 | 51116 |
| MRPS22 | 56945 |
| MRPS35 | 60488 |
| MRTO4 | 51154 |
| MTHFD1 | 4522 |
| MTX1 | 4580 |
| NDUFS6 | 4726 |
| NETO2 | 81831 |
| NLRP1 | 22861 |
| NME1 | 4830 |
| NOC2L | 26155 |
| NOLC1 | 9221 |
| NOP14 | 8602 |
| NOP16 | 51491 |
| NOP2 | 4839 |
| NOSIP | 51070 |
| NPM1 | 4869 |
| NSDHL | 50814 |
| NUDT1 | 4521 |
| NUTF2 | 10204 |
| OR7E37P | 26636 |
| PA2G4 | 5036 |
| PAMR1 | 25891 |
| PCTK1 | 5127 |
| PDCD5 | 9141 |
| PDSS1 | 23590 |
| PES1 | 23481 |
| PGD | 5226 |
| PHB | 5245 |
| PKM2 | 5315 |
| POLD2 | 5425 |
| POLDIP2 | 26073 |
| POLR1C | 9533 |
| POLR1E | 64425 |
| POLR2F | 5435 |
| POLR2H | 5437 |
| POP7 | 10248 |
| PPIH | 10465 |
| PPM1G | 5496 |
| PPP1CA | 5499 |
| PPP4C | 5531 |
| PRDX1 | 5052 |
| PRMT5 | 10419 |
| PSMA5 | 5686 |
| PSMA7 | 5688 |
| PSMB3 | 5691 |
| PSMB4 | 5692 |
| PSMB5 | 5693 |
| PSMC1 | 5700 |
| PSMC3 | 5702 |
| PSMC4 | 5704 |
| PSMD1 | 5707 |
| PSMD2 | 5708 |
| PSMD3 | 5709 |
| PSMD4 | 5710 |
| PSMD8 | 5714 |
| PSME3 | 10197 |
| PTRH2 | 51651 |
| PUF60 | 22827 |
| PUS1 | 80324 |
| RAMP2 | 10266 |
| RANGAP1 | 5905 |
| RBMX2 | 51634 |
| RDBP | 7936 |
| RPL39L | 116832 |
| RPP21 | 79897 |
| RPP38 | 10557 |
| RPS21 | 6227 |
| RPSA | 3921 |
| RRS1 | 23212 |
| RUVBL1 | 8607 |
| RUVBL2 | 10856 |
| SCRIB | 23513 |
| SEMA3G | 56920 |
| SHFM1 | 7979 |
| SIVA1 | 10572 |
| SLC35F2 | 54733 |
| SLC5A6 | 8884 |
| SMARCD2 | 6603 |
| SNED1 | 25992 |
| SNRPB | 6628 |
| SNRPC | 6631 |
| SNRPD2 | 6633 |
| SNRPD3 | 6634 |
| SNRPF | 6636 |
| SRM | 6723 |
| STARD8 | 9754 |
| STIP1 | 10963 |
| STOML2 | 30968 |
| STRA13 | 201254 |
| STYXL1 | 51657 |
| SUPV3L1 | 6832 |
| TARBP2 | 6895 |
| TBCE | 6905 |
| TBRG4 | 9238 |
| TFDP1 | 7027 |
| TIMM10 | 26519 |
| TKT | 7086 |
| TMEM177 | 80775 |
| TOMM22 | 56993 |
| TOMM34 | 10953 |
| TPI1 | 7167 |
| TPT1 | 7178 |
| TRAP1 | 10131 |
| TREX2 | 11219 |
| TSSC1 | 7260 |
| TUBA3C | 7278 |
| TUBB2C | 10383 |
| TUFM | 7284 |
| UCHL3 | 7347 |
| UFD1L | 7353 |
| UQCRH | 7388 |
| VDAC2 | 7417 |
| WDR12 | 55759 |
| WDR18 | 57418 |
| WDR74 | 54663 |
| WDR77 | 79084 |
| XRCC6 | 2547 |
| YARS | 8565 |
| YBX1 | 4904 |
| ZBTB16 | 7704 |
| ZNF259 | 8882 |
| ZNF593 | 51042 |

TC 28

| | |
|---|---|
| ABCG1 | 9619 |
| ARHGAP19 | 84986 |
| BHLHE41 | 79365 |
| BLMH | 642 |
| BRIP1 | 83990 |
| C10ORF116 | 10974 |
| C1ORF2 | 10712 |
| C2ORF44 | 80304 |
| CAD | 790 |
| CCNJ | 54619 |
| CD63 | 967 |
| CIDEB | 27141 |
| COPS7B | 64708 |
| CRYL1 | 51084 |
| CST3 | 1471 |
| DBN1 | 1627 |
| DCLRE1A | 9937 |
| DDX11 | 1663 |
| DDX52 | 11056 |
| DHX35 | 60625 |
| EFNA4 | 1945 |
| FADS1 | 3992 |
| FZD2 | 2535 |
| GTF2IRD1 | 9569 |
| GTPBP8 | 29083 |
| H1FX | 8971 |
| HERPUD1 | 9709 |
| HMGA2 | 8091 |
| INTS7 | 25896 |
| KIAA0040 | 9674 |
| KLHDC3 | 116138 |
| LAPTM4B | 55353 |
| LOC80154 | 80154 |
| MAN2B2 | 23324 |
| MARCH2 | 51257 |
| MDC1 | 9656 |
| MNAT1 | 4331 |
| MORC2 | 22880 |
| NFRKB | 4798 |
| NMU | 10874 |
| NOL9 | 79707 |
| NUCB1 | 4924 |
| NUFIP1 | 26747 |
| NUPR1 | 26471 |
| PHGDH | 26227 |
| PIK3IP1 | 113791 |
| PLAGL2 | 5326 |
| POLG2 | 11232 |
| PPP2R5D | 5528 |
| RBM15B | 29890 |
| RNF8 | 9025 |
| SARS2 | 54938 |
| SH3TC1 | 54436 |
| SLC7A11 | 23657 |
| SMARCB1 | 6598 |
| SMARCD1 | 6602 |
| SMPDL3A | 10924 |
| SOX12 | 6666 |
| SPATS2 | 65244 |
| TAF1A | 9015 |
| TAPBPL | 55080 |
| TBP | 6908 |
| TCTA | 6988 |
| TGIF2 | 60436 |
| TLR5 | 7100 |
| TMEM176A | 55365 |
| TNFRSF14 | 8764 |
| TTLL4 | 9654 |
| UBE4B | 10277 |
| URB2 | 9816 |
| USP13 | 8975 |
| VWA5A | 4013 |
| WRN | 7486 |
| XPO7 | 23039 |
| ZNF232 | 7775 |

TC 29

| | |
|---|---|
| ABCE1 | 6059 |
| ACSM5 | 54988 |
| ACTL6A | 86 |
| ACTR6 | 64431 |
| ACYP1 | 97 |
| ADNP | 23394 |
| ANP32E | 81611 |
| APTX | 54840 |
| BCLAF1 | 9774 |
| BUB3 | 9184 |
| C12ORF11 | 55726 |
| C12ORF41 | 54934 |
| C16ORF80 | 29105 |
| C17ORF71 | 55181 |
| C1ORF77 | 26097 |
| C1ORF9 | 51430 |
| CAND1 | 55832 |
| CASP8AP2 | 9994 |
| CBX1 | 10951 |
| CBX3 | 11335 |
| CCDC41 | 51134 |
| CDK2AP1 | 8099 |
| CDK8 | 1024 |
| CENPQ | 55166 |
| CEP135 | 9662 |
| CEP192 | 55125 |
| CEP57 | 9702 |
| CEP76 | 79959 |
| CKAP2 | 26586 |
| CNOT7 | 29883 |
| CPNE1 | 8904 |
| CPSF6 | 11052 |
| CRNKL1 | 51340 |
| CSF2RA | 1438 |
| CSTF3 | 1479 |
| CTCF | 10664 |
| CUL3 | 8452 |
| DAZAP1 | 26528 |
| DCP1A | 55802 |
| DDX47 | 51202 |
| DDX50 | 79009 |
| DEK | 7913 |
| DENR | 8562 |
| DHX15 | 1665 |
| DNM1L | 10059 |
| DUSP12 | 11266 |
| DUT | 1854 |
| E2F6 | 1876 |
| EED | 8726 |
| EIF2C2 | 27161 |
| ELAVL1 | 1994 |
| ERH | 2079 |
| FANCL | 55120 |
| FBXO46 | 23403 |
| FOXK2 | 3607 |
| FUSIP1 | 10772 |
| FXR1 | 8087 |
| GABPB1 | 2553 |
| GTF2E1 | 2960 |
| GTF3C2 | 2976 |
| GTF3C3 | 9330 |
| HAUS6 | 54801 |
| HLTF | 6596 |
| HMGB2 | 3148 |
| HNRNPA3P1 | 10151 |
| HNRNPH3 | 3189 |
| HNRNPR | 10236 |
| HNRNPA1 | 3178 |
| HNRNPC | 3183 |
| HNRNPK | 3190 |
| HTATSF1 | 27336 |
| IFT52 | 51098 |
| ILF3 | 3609 |
| IPO5 | 3843 |
| ISG20L2 | 81875 |
| KDM3A | 55818 |
| KDM5B | 10765 |
| KHDRBS1 | 10657 |
| KIAA0406 | 9675 |
| KLHL7 | 55975 |
| KRR1 | 11103 |
| LRPPRC | 10128 |
| LSM14A | 26065 |
| LTC4S | 4056 |
| MDM1 | 56890 |
| MDN1 | 23195 |
| MEMO1 | 51072 |
| MPHOSPH9 | 10198 |
| MTF2 | 22823 |
| MTMR4 | 9110 |
| MTPAP | 55149 |
| NAE1 | 8883 |
| NAP1L1 | 4673 |
| NCOA6 | 23054 |
| NKRF | 55922 |
| NOC3L | 64318 |
| NUP160 | 23279 |
| NUP43 | 348995 |
| ORC4L | 5000 |
| PAIP1 | 10605 |
| PARG | 8505 |
| PARP2 | 10038 |
| PAXIP1 | 22976 |
| PFAS | 5198 |
| PGAP1 | 80055 |
| PHF16 | 9767 |
| PNN | 5411 |
| POLA1 | 5422 |
| POLR3B | 55703 |
| PPP1CC | 5501 |
| PRPF40A | 55660 |
| PRPSAP2 | 5636 |
| PTBP1 | 5725 |
| PWP1 | 11137 |
| R3HDM1 | 23518 |
| RAD1 | 5810 |
| RBBP4 | 5928 |
| RBBP7 | 5931 |
| RBM14 | 10432 |
| RBM15 | 64783 |
| RBM28 | 55131 |
| RBM8A | 9939 |
| RBMX | 27316 |
| RCN2 | 5955 |
| RFC1 | 5981 |
| RFX7 | 64864 |
| RIN3 | 79890 |
| RMND5A | 64795 |
| RNASEH1 | 246243 |
| RNASEN | 29102 |
| RNF138 | 51444 |
| RNGTT | 8732 |
| RNMT | 8731 |
| RNPS1 | 10921 |
| RPA1 | 6117 |
| RPAP3 | 79657 |
| RRP15 | 51018 |
| RTF1 | 23168 |
| SAP130 | 79595 |
| SART3 | 9733 |
| SEH1L | 81929 |
| SEPHS1 | 22929 |
| SFPQ | 6421 |
| SFRS1 | 6426 |
| SFRS2 | 6427 |
| SFRS3 | 6428 |
| SFRS7 | 6432 |
| SLBP | 7884 |
| SMARCA4 | 6597 |
| SMARCC1 | 6599 |
| SMARCE1 | 6605 |
| SMC3 | 9126 |
| SMC6 | 79677 |
| SMPD4 | 55627 |
| SPAST | 6683 |
| SS18L1 | 26039 |
| SUMO2 | 6613 |
| SUPT16H | 11198 |
| SUZ12 | 23512 |
| SYNCRIP | 10492 |
| TAF11 | 6882 |
| TAF2 | 6873 |
| TARDBP | 23435 |
| TBPL1 | 9519 |
| TCFL5 | 10732 |
| TDG | 6996 |
| TDP1 | 55775 |
| TERF1 | 7013 |
| TEX10 | 54881 |
| THOC2 | 57187 |
| TOPBP1 | 11073 |
| TRA2B | 6434 |
| TRIT1 | 54802 |
| TRMT11 | 60487 |
| TRRAP | 8295 |
| UBA2 | 10054 |
| UBAP2 | 55833 |
| UBE2V2 | 7336 |
| UPF3B | 65109 |
| USP3 | 9960 |
| UTP18 | 51096 |
| WBP11 | 51729 |
| XPO1 | 7514 |
| YTHDF2 | 51441 |
| YWHAQ | 10971 |
| ZBED4 | 9889 |
| ZNF146 | 7705 |
| ZNF184 | 7738 |
| ZNF227 | 7770 |
| ZW10 | 9183 |

TC 30

| | |
|---|---|
| ACD | 65057 |
| AGPAT1 | 10554 |
| ARF5 | 381 |
| ARHGDIA | 396 |
| ASPSCR1 | 79058 |
| ATP13A1 | 57130 |
| ATP13A2 | 23400 |
| BAX | 581 |
| BSG | 682 |
| BTBD2 | 55643 |
| C19ORF72 | 90379 |
| C9ORF86 | 55684 |
| CALR | 811 |
| CARM1 | 10498 |
| CDC2L1 | 984 |
| CENPB | 1059 |
| CIZ1 | 25792 |
| CLPTM1 | 1209 |
| CNOT3 | 4849 |
| COMMD4 | 54939 |
| DEDD | 9191 |
| DNAJC7 | 7266 |
| DOT1L | 84444 |
| DPM2 | 8818 |
| DRAP1 | 10589 |
| DULLARD | 23399 |
| EIF4G1 | 1981 |
| ERI3 | 79033 |
| FASN | 2194 |
| GANAB | 23193 |
| GBL | 64223 |
| GNB2 | 2783 |
| GPSN2 | 9524 |
| GRINA | 2907 |
| GTF2F1 | 2962 |
| GTF2H4 | 2968 |
| HGS | 9146 |
| HRAS | 3265 |
| KDELR1 | 10945 |
| MAP1S | 55201 |
| MCRS1 | 10445 |
| MED15 | 51586 |
| MMS19 | 64210 |
| MYBBP1A | 10514 |
| NCBP1 | 4686 |
| NELF | 26012 |
| NFYC | 4802 |
| OBFC2B | 79035 |
| PKN1 | 5585 |
| POM121 | 9883 |
| PRKCSH | 5589 |
| PSENEN | 55851 |
| PWP2 | 5822 |
| RAB35 | 11021 |
| RAB5C | 5878 |
| RAD23A | 5886 |
| RBM42 | 79171 |
| RNF220 | 55182 |
| SBF1 | 6305 |
| SCAMP4 | 113178 |
| SEC61A1 | 29927 |
| SENP3 | 26168 |
| SLC25A1 | 6576 |
| SLC4A2 | 6522 |
| STRN4 | 29888 |
| TAF6 | 6878 |
| TRAPPC3 | 27095 |
| UROS | 7390 |
| WBSCR16 | 81554 |
| WDR8 | 49856 |
| XAB2 | 56949 |

TC 31

| | |
|---|---|
| ACOT8 | 10005 |
| AGBL5 | 60509 |
| AP1S1 | 1174 |
| ARD1A | 8260 |
| ARHGEF3 | 50650 |
| ARL6IP4 | 51329 |
| ASCL2 | 430 |
| ATP5D | 513 |
| ATP6V1F | 9296 |
| AURKAIP1 | 54998 |
| AZI1 | 22994 |
| BCL7C | 9274 |
| BOP1 | 23246 |
| C10ORF2 | 56652 |
| C17ORF90 | 339229 |
| C19ORF60 | 55049 |
| C1ORF35 | 79169 |
| C20ORF27 | 54976 |
| CCDC51 | 79714 |
| CCDC94 | 55702 |
| CDK5 | 1020 |
| CHMP1A | 5119 |
| CLPP | 8192 |
| CTNNBL1 | 56259 |
| DIXDC1 | 85458 |
| DNAJB4 | 11080 |
| DOK5 | 55816 |
| DPH2 | 1802 |
| EML1 | 2009 |
| ENDOG | 2021 |
| EPB41L3 | 23136 |
| ERP29 | 10961 |
| FAT4 | 79633 |
| GIPC1 | 10755 |
| GLTPD1 | 80772 |
| GMPPA | 29926 |
| GPS1 | 2873 |
| HSPBP1 | 23640 |
| INO80B | 83444 |
| ISOC2 | 79763 |
| LMAN2 | 10960 |
| LYPLA2 | 11313 |
| MACROD1 | 28992 |
| MAGMAS | 51025 |
| MAP2K2 | 5605 |
| MAZ | 4150 |
| MBNL2 | 10150 |
| MECR | 51102 |
| MED20 | 9477 |
| MKNK1 | 8569 |
| MPG | 4350 |
| MRPL28 | 10573 |
| MRPS34 | 65993 |
| NFKBIB | 4793 |
| NTHL1 | 4913 |
| OTUB1 | 55611 |
| PDAP1 | 11333 |
| PDCD11 | 22984 |
| PET112L | 5188 |
| PEX10 | 5192 |
| PFDN6 | 10471 |
| PPP2R1A | 5518 |
| PPP2R4 | 5524 |
| PPP5C | 5536 |
| PQBP1 | 10084 |
| PRPF31 | 26121 |
| PSMD13 | 5719 |
| PTGES2 | 80142 |
| PYCRL | 65263 |
| RALY | 22913 |
| RNF126 | 55658 |
| RRP7A | 27341 |
| SAPS1 | 22870 |
| SETD8 | 387893 |
| SIGMAR1 | 10280 |
| SIPA1L1 | 26037 |
| SLC1A5 | 6510 |
| SLC8A1 | 6546 |
| SMG5 | 23381 |
| SNRNP35 | 11066 |
| STX10 | 8677 |
| TCEB2 | 6923 |
| TEX264 | 51368 |
| THOP1 | 7064 |
| TIMM17B | 10245 |
| TIMM44 | 10469 |
| TMEM160 | 54958 |
| TSR2 | 90121 |
| WDR46 | 9277 |
| ZNF576 | 79177 |

TC 32

| | |
|---|---|
| ACOT13 | 55856 |
| AIFM1 | 9131 |
| APEH | 327 |
| APOO | 79135 |
| ATP5B | 506 |
| ATP5C1 | 509 |
| ATP5G1 | 516 |
| ATP5G3 | 518 |
| ATP5H | 10476 |
| ATP5I | 521 |
| ATP5J | 522 |
| ATP5L | 10632 |
| ATP5O | 539 |
| ATP6V0B | 533 |
| C12ORF10 | 60314 |
| C14ORF1 | 11161 |
| C19ORF53 | 28974 |
| C19ORF56 | 51398 |
| C3ORF75 | 54859 |
| CCDC56 | 28958 |
| CHCHD2 | 51142 |
| CHCHD8 | 51287 |
| CMAS | 55907 |
| CNPY2 | 10330 |
| COPZ1 | 22818 |
| COQ3 | 51805 |
| COX17 | 10063 |
| COX4I1 | 1327 |
| COX5B | 1329 |
| COX6B1 | 1340 |
| COX6C | 1345 |
| COX7A2 | 1347 |
| COX7A2L | 9167 |
| COX7B | 1349 |
| COX7C | 1350 |
| COX8A | 1351 |
| CS | 1431 |
| DCTN3 | 11258 |
| DCXR | 51181 |
| DDT | 1652 |
| DPH5 | 51611 |
| DRG1 | 4733 |
| EIF2B2 | 8892 |
| EIF3K | 27335 |
| EXOSC7 | 23016 |
| FAM96B | 51647 |
| FH | 2271 |
| FIBP | 9158 |
| FXN | 2395 |
| HADH | 3033 |
| HBXIP | 10542 |
| HINT1 | 3094 |
| HSBP1 | 3281 |
| HSD17B10 | 3028 |
| HYPK | 25764 |
| ICT1 | 3396 |
| IDI1 | 3422 |
| JTB | 10899 |
| LSM3 | 27258 |
| LYRM4 | 57128 |
| MDH1 | 4190 |
| MDH2 | 4191 |
| MKKS | 8195 |
| MPHOSPH6 | 10200 |
| MRPL16 | 54948 |
| MRPL22 | 29093 |
| MRPL33 | 9553 |
| MRPL34 | 64981 |
| MRPL4 | 51073 |
| MRPL46 | 26589 |
| MRPL49 | 740 |
| MRPS14 | 63931 |
| MRPS28 | 28957 |
| MRPS33 | 51650 |
| MRPS7 | 51081 |
| NDUFA1 | 4694 |
| NDUFA10 | 4705 |
| NDUFA13 | 51079 |
| NDUFA3 | 4696 |
| NDUFA4 | 4697 |
| NDUFA6 | 4700 |
| NDUFA7 | 4701 |
| NDUFA8 | 4702 |
| NDUFA9 | 4704 |
| NDUFAB1 | 4706 |
| NDUFAF4 | 29078 |
| NDUFB11 | 54539 |
| NDUFB2 | 4708 |
| NDUFB3 | 4709 |
| NDUFB4 | 4710 |
| NDUFB6 | 4712 |
| NDUFB7 | 4713 |
| NDUFC1 | 4717 |
| NDUFC2 | 4718 |
| NDUFS1 | 4719 |
| NDUFS3 | 4722 |
| NDUFS4 | 4724 |
| NDUFS5 | 4725 |
| NDUFS8 | 4728 |
| NDUFV2 | 4729 |
| NEDD8 | 4738 |
| NHP2 | 55651 |
| NHP2L1 | 4809 |
| NIT2 | 56954 |
| NOD1 | 10392 |
| NOTCH4 | 4855 |
| OXSM | 54995 |
| PARK7 | 11315 |
| PCBD1 | 5092 |
| PCCB | 5096 |
| PDHA1 | 5160 |
| PHB2 | 11331 |
| POLR2I | 5438 |
| POLR2J | 5439 |
| POLR3K | 51728 |
| PPA2 | 27068 |
| PSMB6 | 5694 |
| PXMP2 | 5827 |
| ROBLD3 | 28956 |
| RPA3 | 6119 |
| SAMM50 | 25813 |
| SEC13 | 6396 |
| SF3B5 | 83443 |
| SLC25A11 | 8402 |
| SLC35B1 | 10237 |
| SNRNP25 | 79622 |
| SOD1 | 6647 |
| SUCLG1 | 8802 |
| TIMM13 | 26517 |
| TIMM8B | 26521 |
| TMEM106C | 79022 |
| TMEM147 | 10430 |
| TRIAP1 | 51499 |
| UBE2M | 9040 |
| UBL5 | 59286 |
| UCRC | 29796 |
| UQCR | 10975 |
| UQCRC1 | 7384 |
| UQCRFS1 | 7386 |
| UQCRQ | 27089 |
| UXT | 8409 |

TC 33

| | |
|---|---|
| ADAMTSL3 | 57188 |
| ALDH1A1 | 216 |
| ALG3 | 10195 |
| ANK2 | 287 |
| ARHGAP24 | 83478 |
| BACE1 | 23621 |
| BDH2 | 56898 |
| BHMT2 | 23743 |
| C16ORF45 | 89927 |
| C5ORF23 | 79614 |
| C5ORF4 | 10826 |
| C6ORF108 | 10591 |
| CALCOCO1 | 57658 |
| CCDC46 | 201134 |
| CDO1 | 1036 |
| CITED2 | 10370 |
| CPE | 1363 |
| CYB5R3 | 1727 |
| DAAM2 | 23500 |
| EDIL3 | 10085 |
| EIF4EBP1 | 1978 |
| ENPP2 | 5168 |
| F8 | 2157 |
| FAM127A | 8933 |
| FBXL7 | 23194 |
| FRY | 10129 |
| GHR | 2690 |
| GPR172A | 79581 |
| GPX3 | 2878 |
| HLF | 3131 |
| HMBS | 3145 |
| HMGA1 | 3159 |
| HSPA12A | 259217 |
| IFRD2 | 7866 |
| IL11RA | 3590 |
| IQSEC1 | 9922 |
| ITPR1 | 3708 |
| KCNJ8 | 3764 |
| LOC643287 | 643287 |
| LRFN4 | 78999 |
| MAN1C1 | 57134 |
| MEIS3P1 | 4213 |
| NDN | 4692 |
| OSBPL1A | 114876 |
| PCDH17 | 27253 |
| PDE2A | 5138 |
| PDIA4 | 9601 |
| PER1 | 5187 |
| PIK3R1 | 5295 |
| PKIG | 11142 |
| PLA2G4C | 8605 |
| PTMAP7 | 326626 |
| RAI2 | 10742 |
| RCAN2 | 10231 |
| RPS2 | 6187 |
| RUNX1T1 | 862 |
| SATB1 | 6304 |
| SDC2 | 6383 |
| SDF2L1 | 23753 |
| SEPP1 | 6414 |
| SGCD | 6444 |
| SLC16A4 | 9122 |
| SLC29A2 | 3177 |
| SLC7A5 | 8140 |
| SOCS2 | 8835 |
| TACC1 | 6867 |
| TEAD4 | 7004 |
| TGFBR3 | 7049 |
| TRAF4 | 9618 |
| TTLL12 | 23170 |
| UTRN | 7402 |
| WWC3 | 55841 |
| XPC | 7508 |
| YKT6 | 10652 |
| ZBTB20 | 26137 |

TC 34

| | |
|---|---|
| ACACB | 32 |
| ADK | 132 |
| APBB3 | 10307 |
| ARHGEF17 | 9828 |
| ARNTL2 | 56938 |
| ASL | 435 |
| BID | 637 |
| C20ORF24 | 55969 |
| CASP3 | 836 |
| CEBPG | 1054 |
| CHD3 | 1107 |
| COQ2 | 27235 |
| CRY2 | 1408 |
| CSTB | 1476 |
| DBI | 1622 |
| DPP3 | 10072 |
| DYNC2H1 | 79659 |
| ENO1 | 2023 |
| ERO1L | 30001 |
| ESRP1 | 54845 |
| ETHE1 | 23474 |
| EXOC7 | 23265 |
| F11R | 50848 |
| FABP5 | 2171 |
| FAM60A | 58516 |
| FAM65A | 79567 |
| FBXO17 | 115290 |
| FGFR1 | 2260 |
| FRAT2 | 23401 |
| GLRX5 | 51218 |
| GSK3B | 2932 |
| HDGF | 3068 |
| HTATIP2 | 10553 |
| IRAK1 | 3654 |
| KCNK3 | 3777 |
| KCTD5 | 54442 |
| LDHA | 3939 |
| LOC201229 | 201229 |
| LRRC16A | 55604 |
| LRRC59 | 55379 |
| MAP3K12 | 7786 |
| METTL7A | 25840 |
| MGAT4B | 11282 |
| MLX | 6945 |
| NFASC | 23114 |
| NP | 4860 |
| ORMDL2 | 29095 |
| PABPC3 | 5042 |
| PERP | 64065 |
| PHF1 | 5252 |
| PPA1 | 5464 |
| PPCS | 79717 |
| PPIF | 10105 |
| PPPDE2 | 27351 |
| PRDX4 | 10549 |
| PREP | 5550 |
| PRR13 | 54458 |
| PTMA | 5757 |
| RP6- | 51765 |
| 213H19.1 | |
| SGSM2 | 9905 |
| SLC25A5 | 292 |
| SPCS3 | 60559 |
| STRADA | 92335 |
| TALDO1 | 6888 |
| TENC1 | 23371 |
| TFRC | 7037 |
| TPD52 | 7163 |
| TSPYL2 | 64061 |
| TXN | 7295 |

TC 35

| | |
|---|---|
| EEF1B2 | 1933 |
| EEF1D | 1936 |
| EEF1G | 1937 |
| EIF3E | 3646 |
| EIF3G | 8666 |
| EIF3H | 8667 |
| EIF3L | 51386 |
| EIF3F | 8665 |
| EIF3D | 8664 |
| FAU | 2197 |
| GNB2L1 | 10399 |
| IGBP1 | 3476 |
| IMPDH2 | 3615 |
| LOC391132 | 391132 |
| LOC399804 | 399804 |
| NACA | 4666 |
| QARS | 5859 |
| RPL10L | 140801 |
| RPL11 | 6135 |
| RPL12 | 6136 |
| RPL13 | 6137 |
| RPL13A | 23521 |
| RPL14 | 9045 |
| RPL15P22 | 100130624 |
| RPL17 | 6139 |
| RPL18 | 6141 |
| RPL18A | 6142 |
| RPL18P11 | 390612 |
| RPL19 | 6143 |
| RPL21 | 6144 |
| RPL22 | 6146 |
| RPL23 | 9349 |
| RPL23A | 6147 |
| RPL24 | 6152 |
| RPL26P37 | 441533 |
| RPL27 | 6155 |
| RPL28 | 6158 |
| RPL29 | 6159 |
| RPL3 | 6122 |
| RPL30 | 6156 |
| RPL31 | 6160 |
| RPL32 | 6161 |
| RPL34 | 6164 |
| RPL35 | 11224 |
| RPL36 | 25873 |
| RPL36A | 6173 |
| RPL3P7 | 642741 |
| RPL4 | 6124 |
| RPL5 | 6125 |
| RPL6 | 6128 |
| RPL7 | 6129 |
| RPL7A | 6130 |
| RPL8 | 6132 |
| RPLP0 | 6175 |
| RPLP1 | 6176 |
| RPS10 | 6204 |
| RPS10P5 | 93144 |
| RPS12 | 6206 |
| RPS13 | 6207 |
| RPS14 | 6208 |
| RPS15 | 6209 |
| RPS16 | 6217 |
| RPS17 | 6218 |
| RPS17P5 | 442216 |
| RPS18 | 6222 |
| RPS19 | 6223 |
| RPS20 | 6224 |
| RPS24 | 6229 |
| RPS25 | 6230 |
| RPS28P6 | 728453 |
| RPS29 | 6235 |
| RPS3 | 6188 |
| RPS3A | 6189 |
| RPS4X | 6191 |
| RPS5 | 6193 |
| RPS6 | 6194 |
| RPS7 | 6201 |
| RPS8 | 6202 |
| RPS9 | 6203 |
| SSR2 | 6746 |
| TINP1 | 10412 |
| UBA52 | 7311 |

TC 36

| | |
|---|---|
| ARPC1A | 10552 |
| ATP5F1 | 515 |
| BTF3 | 689 |
| C20ORF30 | 29058 |
| C9ORF46 | 55848 |
| CDK7 | 1022 |
| CDV3 | 55573 |
| COPB2 | 9276 |
| CYB5R4 | 51167 |
| DAD1 | 1603 |
| DCTD | 1635 |
| DSCR3 | 10311 |
| ECHDC1 | 55862 |
| FAM106A | 80039 |
| FLJ23172 | 389177 |
| GDE1 | 51573 |
| GDI2 | 2665 |
| GHITM | 27069 |
| GNG5 | 2787 |
| HEBP2 | 23593 |
| HNRNPF | 3185 |
| HSP90AB1 | 3326 |
| HSPA8 | 3312 |
| M6PR | 4074 |
| MAP1LC3B | 81631 |
| MAPKBP1 | 23005 |
| MAPRE1 | 22919 |
| MGC1 | 84786 |
| MRPL44 | 65080 |
| NDUFB5 | 4711 |
| NOP10 | 55505 |
| NRBF2 | 29982 |
| OAZ1 | 4946 |
| PCBP1 | 5093 |
| PCNXL2 | 80003 |
| PDIA6 | 10130 |
| PGRMC1 | 10857 |
| PNRC2 | 55629 |
| POP4 | 10775 |
| PRDX3 | 10935 |
| PSMA1 | 5682 |
| PSMD9 | 5715 |
| RAB5A | 5868 |
| RAB9A | 9367 |
| RARS | 5917 |
| RBX1 | 9978 |
| RPL10A | 4736 |
| SAR1A | 56681 |
| SDHB | 6390 |
| SDHC | 6391 |
| SDHD | 6392 |
| SEC11A | 23478 |
| SELT | 51714 |
| SLC25A3 | 5250 |
| SNX5 | 27131 |
| SNX7 | 51375 |
| SPCS1 | 28972 |
| SPCS2 | 9789 |
| SUMO3 | 6612 |
| TAF9 | 6880 |
| TM9SF2 | 9375 |
| TMEM111 | 55831 |
| TMEM70 | 54968 |
| TOMM20 | 9804 |
| UBE2D3 | 7323 |
| UQCRC2 | 7385 |
| VDAC1 | 7416 |

TC 37

| | |
|---|---|
| ACTR2 | 10097 |
| ADAM9 | 8754 |
| ARF4 | 378 |
| ARF6 | 382 |
| ARL8B | 55207 |
| ARPC3 | 10094 |
| ARPC5 | 10092 |
| ATP1B2 | 482 |
| BZW1 | 9689 |
| CAB39 | 51719 |
| CAPZA2 | 830 |
| CD164 | 8763 |
| CHMP2B | 25978 |
| CMPK1 | 51727 |
| CMTM6 | 54918 |
| CROCC | 9696 |
| DAZAP2 | 9802 |
| DDX3X | 1654 |
| DERL1 | 79139 |
| ETF1 | 2107 |
| FAM49B | 51571 |
| G3BP1 | 10146 |
| GCA | 25801 |
| GNAI3 | 2773 |
| GTF2B | 2959 |
| LRDD | 55367 |
| MAT2B | 27430 |
| MMADHC | 27249 |
| MOBKL1B | 55233 |
| NAT13 | 80218 |
| NCK1 | 4690 |
| NCOA4 | 8031 |
| NFE2L2 | 4780 |
| NRAS | 4893 |
| PDCD6IP | 10015 |
| PSEN1 | 5663 |
| PTP4A2 | 8073 |
| RAB1A | 5861 |
| RHOA | 387 |
| SCP2 | 6342 |
| SEPT2 | 4735 |
| SH3GLB1 | 51100 |
| SNX2 | 6643 |
| SNX3 | 8724 |
| SSR1 | 6745 |
| SUCLG2 | 8801 |
| SYPL1 | 6856 |
| TAZ | 6901 |
| TBL1XR1 | 79718 |
| TMED5 | 50999 |
| TMEM30A | 55754 |
| TMEM50B | 757 |
| TMEM9B | 56674 |
| TMOD3 | 29766 |
| TMX1 | 81542 |
| VAMP3 | 9341 |
| VPS24 | 51652 |
| WDTC1 | 23038 |
| WTAP | 9589 |
| YIPF5 | 81555 |
| YWHAZ | 7534 |

TC 38

| | |
|---|---|
| ACOT9 | 23597 |
| AHR | 196 |
| AK2 | 204 |
| APLP1 | 333 |
| ARPC2 | 10109 |
| BCL7A | 605 |
| C7ORF23 | 79161 |
| CALU | 813 |
| CAP1 | 10487 |
| CAST | 831 |
| CCDC109B | 55013 |
| CD55 | 1604 |
| CD58 | 965 |
| CHST10 | 9486 |
| CKLF | 51192 |
| COPG2IT1 | 53844 |
| COTL1 | 23406 |
| DUSP26 | 78986 |
| FAM125B | 89853 |
| FHL2 | 2274 |
| FLJ22184 | 80164 |
| HIP1R | 9026 |
| IFNGR1 | 3459 |
| IFNGR2 | 3460 |
| IL10RB | 3588 |
| IQGAP1 | 8826 |
| JAKMIP2 | 9832 |
| JOSD1 | 9929 |
| LY75 | 4065 |
| MICAL2 | 9645 |
| MYD88 | 4615 |
| MYL12A | 10627 |
| MYOF | 26509 |
| NCAM1 | 4684 |
| NMI | 9111 |
| PACRG | 135138 |
| PLSCR1 | 5359 |
| POMT1 | 10585 |
| PPIC | 5480 |
| RALB | 5899 |
| RND2 | 8153 |
| RNF19B | 127544 |
| SARM1 | 23098 |
| SEMA3C | 10512 |
| SHC2 | 25759 |
| STEAP1 | 26872 |
| TAX1BP3 | 30851 |
| TES | 26136 |
| TGIF1 | 7050 |
| TMEM49 | 81671 |
| TNFAIP8 | 25816 |
| TRAM1 | 23471 |

TC 39

| | |
|---|---|
| ABCG2 | 9429 |
| ACVRL1 | 94 |
| ADAMTS5 | 11096 |
| ADM | 133 |
| ANGPT2 | 285 |
| APOLD1 | 81575 |
| ARAP3 | 64411 |
| BTG1 | 694 |
| CCDC102B | 79839 |
| CCND1 | 595 |
| CDH13 | 1012 |
| COL21A1 | 81578 |
| CP | 1356 |
| CRIP2 | 1397 |
| CX3CL1 | 6376 |
| DPP4 | 1803 |
| EGLN3 | 112399 |
| ENPEP | 2028 |
| ESM1 | 11082 |
| FAM38B | 63895 |
| FHL5 | 9457 |
| FMO3 | 2328 |
| GALNT14 | 79623 |
| HBA1 | 3039 |
| HBB | 3043 |
| HEY2 | 23493 |
| ICAM2 | 3384 |
| INHBB | 3625 |
| KCNJ15 | 3772 |
| KDR | 3791 |
| LEPREL1 | 55214 |
| LPCAT1 | 79888 |
| LPL | 4023 |
| MOSC2 | 54996 |
| NDUFA4L2 | 56901 |
| NOL3 | 8996 |
| OLFML2A | 169611 |
| PCDH12 | 51294 |
| PCTK3 | 5129 |
| PLA1A | 51365 |
| PLVAP | 83483 |
| PRCP | 5547 |
| RASIP1 | 54922 |
| RERGL | 79785 |
| RHOBTB1 | 9886 |
| RRAD | 6236 |
| SCARF1 | 8578 |
| SLC27A3 | 11000 |
| SLC47A1 | 55244 |
| SNX29 | 92017 |
| SOX17 | 64321 |
| SOX18 | 54345 |
| STC1 | 6781 |
| TPPP3 | 51673 |
| TRIOBP | 11078 |
| TSPAN12 | 23554 |
| UNC5B | 219699 |
| VEGFA | 7422 |

TC 40

| | |
|---|---|
| A2M | 2 |
| ABCA8 | 10351 |
| ADAMTS1 | 9510 |
| ADH1B | 125 |
| AOC3 | 8639 |
| APLNR | 187 |
| AQP1 | 358 |
| ASPA | 443 |
| C10ORF10 | 11067 |
| C13ORF15 | 28984 |
| C6ORF145 | 221749 |
| CALCRL | 10203 |
| CCL14 | 6358 |
| CD34 | 947 |
| CD36 | 948 |
| CDH5 | 1003 |
| CLDN5 | 7122 |
| CLEC3B | 7123 |
| CMAH | 8418 |
| CRYAB | 1410 |
| CX3CR1 | 1524 |
| CXCL12 | 6387 |
| DARC | 2532 |
| EDN1 | 1906 |
| EDNRB | 1910 |
| EGR1 | 1958 |
| ELN | 2006 |
| ELTD1 | 64123 |
| EMCN | 51705 |
| EPAS1 | 2034 |
| ERG | 2078 |
| FBLN5 | 10516 |
| FHL1 | 2273 |
| FMO2 | 2327 |
| FOSB | 2354 |
| FRZB | 2487 |
| FXYD1 | 5348 |
| GADD45B | 4616 |
| GAS6 | 2621 |
| GJA4 | 2701 |
| GNG11 | 2791 |
| GPR116 | 221395 |
| GRK5 | 2869 |
| HSPB8 | 26353 |
| HYAL2 | 8692 |
| ITGA7 | 3679 |
| ITIH5 | 80760 |
| ITM2A | 9452 |
| JUN | 3725 |
| KIAA1462 | 57608 |
| LIMS2 | 55679 |
| LMOD1 | 25802 |
| LOH3CR2A | 29931 |
| LRRC32 | 2615 |
| LYVE1 | 10894 |
| MAOB | 4129 |
| MCAM | 4162 |
| MMRN2 | 79812 |
| NR2F1 | 7025 |
| P2RY14 | 9934 |
| PALMD | 54873 |
| PDGFD | 80310 |
| PDK4 | 5166 |
| PLN | 5350 |
| PNRC1 | 10957 |
| PPAP2A | 8611 |
| PPAP2B | 8613 |
| PPP1R12B | 4660 |
| PRELP | 5549 |
| PRKCH | 5583 |
| PTGDS | 5730 |
| PTPRB | 5787 |
| PTPRM | 5797 |
| RAMP3 | 10268 |
| RASL12 | 51285 |
| RGS5 | 8490 |
| RHOB | 388 |
| RPS6KA2 | 6196 |
| S1PR1 | 1901 |
| SDPR | 8436 |
| SELP | 6403 |
| SLCO2A1 | 6578 |
| SLIT3 | 6586 |
| SORBS1 | 10580 |
| STEAP4 | 79689 |
| SYNPO | 11346 |
| TEK | 7010 |
| TIE1 | 7075 |
| TSC22D3 | 1831 |
| VWF | 7450 |

TC 41

| | |
|---|---|
| BNC2 | 54796 |
| C7 | 730 |
| C7ORF58 | 79974 |
| CALD1 | 800 |
| CD81 | 975 |
| COL6A2 | 1292 |
| COPZ2 | 51226 |
| COX7A1 | 1346 |
| CYBRD1 | 79901 |
| DCHS1 | 8642 |
| DDR2 | 4921 |
| DPT | 1805 |
| EFEMP2 | 30008 |
| EHD2 | 30846 |
| EMILIN1 | 11117 |
| FYN | 2534 |
| GLT8D2 | 83468 |
| GPR124 | 25960 |
| GUCY1A3 | 2982 |
| GUCY1B3 | 2983 |
| GYPC | 2995 |
| HSPG2 | 3339 |
| IFFO1 | 25900 |
| IGFBP4 | 3487 |
| ILK | 3611 |
| ISLR | 3671 |
| JAM2 | 58494 |
| JAM3 | 83700 |
| KANK2 | 25959 |
| KCTD12 | 115207 |
| LAMB2 | 3913 |
| LDB2 | 9079 |
| LMO2 | 4005 |
| LRP1 | 4035 |
| MEF2C | 4208 |
| MEIS1 | 4211 |
| MFAP4 | 4239 |
| MOXD1 | 26002 |
| MRC2 | 9902 |
| MXRA8 | 54587 |
| OLFML3 | 56944 |
| PCDHGC3 | 5098 |
| PDE1A | 5136 |
| PDGFRB | 5159 |
| PGCP | 10404 |
| PLAT | 5327 |
| PLXDC1 | 57125 |
| PTGIS | 5740 |
| PTRF | 284119 |
| RBMS3 | 27303 |
| RBPMS | 11030 |
| SLIT2 | 9353 |
| SPARCL1 | 8404 |
| SPRY1 | 10252 |
| TCF4 | 6925 |
| TIMP3 | 7078 |
| TNS1 | 7145 |
| ZCCHC24 | 219654 |
| ZNF423 | 23090 |

TC 42

| | |
|---|---|
| ADCY7 | 113 |
| ARHGAP29 | 9411 |
| ARL6IP5 | 10550 |
| ASAH1 | 427 |
| BNIP3L | 665 |
| C16ORF59 | 80178 |
| C3ORF64 | 285203 |
| C9ORF45 | 81571 |
| CIB2 | 10518 |
| COQ10B | 80219 |
| CREM | 1390 |
| CRIM1 | 51232 |
| CTBS | 1486 |
| DEGS1 | 8560 |
| DPYD | 1806 |
| DSE | 29940 |
| EPS8 | 2059 |
| F2R | 2149 |
| FKBPL | 63943 |
| GNG12 | 55970 |
| GPR137B | 7107 |
| ITGAV | 3685 |
| JAG1 | 182 |
| KIAA0247 | 9766 |
| KLF10 | 7071 |
| LAMP2 | 3920 |
| LAPTM4A | 9741 |
| LIMS1 | 3987 |
| LRRC20 | 55222 |
| MARCKS | 4082 |
| MFSD1 | 64747 |
| NDEL1 | 81565 |
| NOC4L | 79050 |
| P2RY5 | 10161 |
| PATZ1 | 23598 |
| PELO | 53918 |
| PLS3 | 5358 |
| POLE | 5426 |
| PPT1 | 5538 |
| PTPRE | 5791 |
| RAB8B | 51762 |
| RAP1A | 5906 |
| RBM4 | 5936 |
| RIN2 | 54453 |
| RNF13 | 11342 |
| SDCBP | 6386 |
| SGPP1 | 81537 |
| SH2B3 | 10019 |
| SMAD7 | 4092 |
| SMYD5 | 10322 |
| SPHK2 | 56848 |
| STX12 | 23673 |
| STX7 | 8417 |
| SWAP70 | 23075 |
| TOP3A | 7156 |
| TRIM8 | 81603 |
| WRAP53 | 55135 |
| XRCC3 | 7517 |
| YAP1 | 10413 |
| ZNF408 | 79797 |

TC 43

| | |
|---|---|
| AKAP2 | 11217 |
| ATAD3A | 55210 |
| ATP10D | 57205 |
| ATXN1 | 6310 |
| BLM | 641 |
| C10ORF26 | 54838 |
| C18ORF1 | 753 |
| CCNF | 899 |
| CCPG1 | 9236 |
| CD302 | 9936 |
| CDC25A | 993 |
| CDC25C | 995 |
| CHAF1A | 10036 |
| CHAF1B | 8208 |
| CREBL2 | 1389 |
| CTSO | 1519 |
| DENND5A | 23258 |
| E2F1 | 1869 |
| EXO1 | 9156 |
| FAM114A2 | 10827 |
| FANCE | 2178 |
| FCHSD2 | 9873 |
| GTSE1 | 51512 |
| ITM2B | 9445 |
| KIF22 | 3835 |
| KIFC1 | 3833 |
| KLF9 | 687 |
| MRPS12 | 6183 |
| MYBL2 | 4605 |
| NR3C1 | 2908 |
| ORC1L | 4998 |
| PION | 54103 |
| PJA2 | 9867 |
| PKD2 | 5311 |
| PKMYT1 | 9088 |
| PLSCR4 | 57088 |
| QKI | 9444 |
| RANBP1 | 5902 |
| RCBTB2 | 1102 |
| RCC1 | 1104 |
| RQCD1 | 9125 |
| SERINC1 | 57515 |
| SH3BGRL | 6451 |
| SLC7A1 | 6541 |
| TFAM | 7019 |
| TOMM40 | 10452 |
| TXNDC15 | 79770 |
| ZEB1 | 6935 |

TC 44

| | |
|---|---|
| ADAM12 | 8038 |
| AEBP1 | 165 |
| ANGPTL2 | 23452 |
| BASP1 | 10409 |
| BGN | 633 |
| CD248 | 57124 |
| CD99 | 4267 |
| COL10A1 | 1300 |
| COL11A1 | 1301 |
| COL16A1 | 1307 |
| COL1A1 | 1277 |
| COL4A2 | 1284 |
| COL5A1 | 1289 |
| COL8A1 | 1295 |
| COL8A2 | 1296 |
| COMP | 1311 |
| CTSK | 1513 |
| CYP1B1 | 1545 |
| DACT1 | 51339 |
| DPYSL3 | 1809 |
| ECM1 | 1893 |
| FAM114A1 | 92689 |
| FAP | 2191 |
| FBLN2 | 2199 |
| FLNA | 2316 |
| FN1 | 2335 |
| GAS1 | 2619 |
| GCDH | 2639 |
| GFPT2 | 9945 |
| GGT5 | 2687 |
| GREM1 | 26585 |
| INHBA | 3624 |
| ITGA5 | 3678 |
| ITGBL1 | 9358 |
| LEPRE1 | 64175 |
| LMCD1 | 29995 |
| LOX | 4015 |
| LOXL1 | 4016 |
| LRRC15 | 131578 |
| MFAP2 | 4237 |
| MFAP5 | 8076 |
| MFGE8 | 4240 |
| MMP11 | 4320 |
| MN1 | 4330 |
| MXRA5 | 25878 |
| NTM | 50863 |
| NUAK1 | 9891 |
| NXN | 64359 |
| PCDH7 | 5099 |
| PCOLCE | 5118 |
| PCSK5 | 5125 |
| PDGFRL | 5157 |
| PDLIM2 | 64236 |
| PDLIM3 | 27295 |
| PDPN | 10630 |
| PLSCR3 | 57048 |
| PMEPA1 | 56937 |
| POSTN | 10631 |
| PRRX1 | 5396 |
| PXDN | 7837 |
| RCN3 | 57333 |
| RGS3 | 5998 |
| SERPINH1 | 871 |
| SFRP4 | 6424 |
| SFXN3 | 81855 |
| SPHK1 | 8877 |
| SPON1 | 10418 |
| SPON2 | 10417 |
| SPSB1 | 80176 |
| SRPX2 | 27286 |
| SULF1 | 23213 |
| TGFB3 | 7043 |
| THBS2 | 7058 |
| THY1 | 7070 |
| TMEM45A | 55076 |
| TNC | 3371 |
| TNFAIP6 | 7130 |
| TNFSF4 | 7292 |
| TPM2 | 7169 |
| TSHZ2 | 128553 |
| TWIST1 | 7291 |
| WISP1 | 8840 |

TC 45

| | |
|---|---|
| ABCA1 | 19 |
| ANTXR1 | 84168 |
| ANXA5 | 308 |
| ASPN | 54829 |
| BCL6 | 604 |
| C17ORF91 | 84981 |
| C4ORF18 | 51313 |
| CD93 | 22918 |
| CDH11 | 1009 |
| CLIC4 | 25932 |
| CNN3 | 1266 |
| COL15A1 | 1306 |
| COL1A2 | 1278 |
| COL3A1 | 1281 |
| COL4A1 | 1282 |
| COL5A2 | 1290 |
| COL6A3 | 1293 |
| COLEC12 | 81035 |
| CRISPLD2 | 83716 |
| CTGF | 1490 |
| DKK3 | 27122 |
| ECM2 | 1842 |
| EDNRA | 1909 |
| EFEMP1 | 2202 |
| EGR2 | 1959 |
| ELK3 | 2004 |
| EMP1 | 2012 |
| FBN1 | 2200 |
| FEZ1 | 9638 |
| FILIP1L | 11259 |
| FSTL1 | 11167 |
| GALNAC4S- | 51363 |
| 6ST | |
| GEM | 2669 |
| GJA1 | 2697 |
| HEG1 | 57493 |
| HTRA1 | 5654 |
| IGFBP7 | 3490 |
| ITGB5 | 3693 |
| KAL1 | 3730 |
| LAMB1 | 3912 |
| LAMC1 | 3915 |
| LBH | 81606 |
| LHFP | 10186 |
| LTBP1 | 4052 |
| LUM | 4060 |
| MGP | 4256 |
| MMP2 | 4313 |
| MSN | 4478 |
| MYLK | 4638 |
| NID1 | 4811 |
| NID2 | 22795 |
| NOTCH2 | 4853 |
| NRP1 | 8829 |
| OLFML1 | 283298 |
| OLFML2B | 25903 |
| PALLD | 23022 |
| PARVA | 55742 |
| PDGFC | 56034 |
| PEA15 | 8682 |
| PMP22 | 5376 |
| PROS1 | 5627 |
| PRSS23 | 11098 |
| RAB31 | 11031 |
| RBMS1 | 5937 |
| RFTN1 | 23180 |
| RGL1 | 23179 |
| RHOQ | 23433 |
| SNAI2 | 6591 |
| SPARC | 6678 |
| SRPX | 8406 |
| STON1 | 11037 |
| TGFB1I1 | 7041 |
| THBS1 | 7057 |
| TIMP2 | 7077 |
| TMEM47 | 83604 |
| TPM1 | 7168 |
| TRIB2 | 28951 |
| VCAN | 1462 |
| VGLL3 | 389136 |
| ZFPM2 | 23414 |

TC 46

| | |
|---|---|
| ARHGEF6 | 9459 |
| ARL4C | 10123 |
| C1ORF54 | 79630 |
| C1R | 715 |
| C1S | 716 |
| C3 | 718 |
| CALHM2 | 51063 |
| CCL2 | 6347 |
| CD59 | 966 |
| CFD | 1675 |
| CFH | 3075 |
| CFI | 3426 |
| CPA3 | 1359 |
| CTSL1 | 1514 |
| CXCL2 | 2920 |
| CYR61 | 3491 |
| DAB2 | 1601 |
| DCN | 1634 |
| DRAM | 55332 |
| DUSP1 | 1843 |
| ENG | 2022 |
| F13A1 | 2162 |
| FCGRT | 2217 |
| FOS | 2353 |
| GLIPR1 | 11010 |
| GPNMB | 10457 |
| IFITM2 | 10581 |
| IFITM3 | 10410 |
| IL1R1 | 3554 |
| JUNB | 3726 |
| KLF6 | 1316 |
| LITAF | 9516 |
| LTBP2 | 4053 |
| LXN | 56925 |
| MAF | 4094 |
| MYH9 | 4627 |
| MYL9 | 10398 |
| NNMT | 4837 |
| PECAM1 | 5175 |
| PLAU | 5328 |
| PSAP | 5660 |
| RARRES2 | 5919 |
| RASSF2 | 9770 |
| RGS2 | 5997 |
| RNASE1 | 6035 |
| RNF130 | 55819 |
| RRAS | 6237 |
| S100A4 | 6275 |
| SERPINE1 | 5054 |
| SERPINF1 | 5176 |
| SERPING1 | 710 |
| SGK1 | 6446 |
| SOCS3 | 9021 |
| STAB1 | 23166 |
| STOM | 2040 |
| TAGLN | 6876 |
| TGFBI | 7045 |
| TGFBR2 | 7048 |
| THBD | 7056 |
| TIMP1 | 7076 |
| TNFRSF1A | 7132 |
| TPSAB1 | 7177 |
| TPSB2 | 64499 |
| UBA7 | 7318 |
| VCAM1 | 7412 |
| VIM | 7431 |
| ZFP36 | 7538 |

TC 47

| | |
|---|---|
| ADAMDEC1 | 27299 |
| AIM2 | 9447 |
| APOBEC3G | 60489 |
| ARHGAP25 | 9938 |
| BANK1 | 55024 |
| BTN2A2 | 10385 |
| BTN3A2 | 11118 |
| CCDC69 | 26112 |
| CCL19 | 6363 |
| CCL3 | 6348 |
| CCL4 | 6351 |
| CCL8 | 6355 |
| CCR2 | 729230 |
| CCR5 | 1234 |
| CCR7 | 1236 |
| CD19 | 930 |
| CD1D | 912 |
| CD247 | 919 |
| CD27 | 939 |
| CD38 | 952 |
| CD3E | 916 |
| CD72 | 971 |
| CD83 | 9308 |
| CD8A | 925 |
| CD96 | 10225 |
| CECR1 | 51816 |
| CLEC2D | 29121 |
| CRTAM | 56253 |
| CST7 | 8530 |
| CTSW | 1521 |
| CXCL11 | 6373 |
| CXCL13 | 10563 |
| CXCL9 | 4283 |
| DEF6 | 50619 |
| DUSP2 | 1844 |
| EAF2 | 55840 |
| FAIM3 | 9214 |
| FAM65B | 9750 |
| FGR | 2268 |
| GNLY | 10578 |
| GPR171 | 29909 |
| GPR18 | 2841 |
| GVIN1 | 387751 |
| GZMA | 3001 |
| GZMB | 3002 |
| GZMK | 3003 |
| HLA-DOB | 3112 |
| HLA-DQA1 | 3117 |
| ICOS | 29851 |
| IDO1 | 3620 |
| IGHD | 3495 |
| IGHM | 3507 |
| IGKV3D- | 28875 |
| 15 | |
| IGKV4-1 | 28908 |
| IGLJ3 | 28831 |
| IGLV3-19 | 28797 |
| IKZF1 | 10320 |
| IL18RAP | 8807 |
| IL2RB | 3560 |
| ITK | 3702 |
| JAK2 | 3717 |
| KLRB1 | 3820 |
| KLRD1 | 3824 |
| KLRK1 | 22914 |
| LAG3 | 3902 |
| LAX1 | 54900 |
| LCK | 3932 |
| LRMP | 4033 |
| MARCH1 | 55016 |
| MS4A1 | 931 |
| NKG7 | 4818 |
| NOD2 | 64127 |
| P2RX5 | 5026 |
| P2RY13 | 53829 |
| PIK3CD | 5293 |
| PIM2 | 11040 |
| POU2AF1 | 5450 |
| PPP1R16B | 26051 |
| PRF1 | 5551 |
| PRKCB | 5579 |
| PTPN7 | 5778 |
| PVRIG | 79037 |
| RASGRP1 | 10125 |
| RHOH | 399 |
| RUNX3 | 864 |
| SAMHD1 | 25939 |
| SELL | 6402 |
| SIRPG | 55423 |
| SLAMF1 | 6504 |
| SP140 | 11262 |
| STAT4 | 6775 |
| STAT5A | 6776 |
| SYK | 6850 |
| TARP | 445347 |
| TCL1A | 8115 |
| TLR8 | 51311 |
| TNFRSF17 | 608 |
| TRAF1 | 7185 |
| TRAF3IP3 | 80342 |
| TRAT1 | 50852 |
| TRGC2 | 6967 |
| VNN2 | 8875 |
| XCL1 | 6375 |

TC 48

| | |
|---|---|
| AOAH | 313 |
| APOB48R | 55911 |
| ARHGAP4 | 393 |
| BTK | 695 |
| BTN3A1 | 11119 |
| C17ORF60 | 284021 |
| CARD9 | 64170 |
| CCL21 | 6366 |
| CCL23 | 6368 |
| CD180 | 4064 |
| CD40 | 958 |
| CD7 | 924 |
| CLEC10A | 10462 |
| CMKLR1 | 1240 |
| CR1 | 1378 |
| CSF3R | 1441 |
| CTLA4 | 1493 |
| CXCR6 | 10663 |
| CYTH4 | 27128 |
| DENND1C | 79958 |
| DENND3 | 22898 |
| DOK2 | 9046 |
| DPEP2 | 64174 |
| FCN1 | 2219 |
| FES | 2242 |
| FMNL1 | 752 |
| GMIP | 51291 |
| GPSM3 | 63940 |
| GZMH | 2999 |
| HK3 | 3101 |
| IGH@ | 3492 |
| IGHA1 | 3493 |
| IGHV3OR16-6 | 647187 |
| IL16 | 3603 |
| IL21R | 50615 |
| INPP5D | 3635 |
| ITGAL | 3683 |
| ITGAX | 3687 |
| LAT | 27040 |
| LILRA6 | 79168 |
| LILRB4 | 11006 |
| LSP1 | 4046 |
| LTB | 4050 |
| LY9 | 4063 |
| MAP4K1 | 11184 |
| MGC29506 | 51237 |
| PSTPIP1 | 9051 |
| PTK2B | 2185 |
| PTPRCAP | 5790 |
| SELPLG | 6404 |
| SH2D1A | 4068 |
| SIPA1 | 6494 |
| SLAMF7 | 57823 |
| SPI1 | 6688 |
| STX11 | 8676 |
| TMEM149 | 79713 |
| TRPV2 | 51393 |
| VAV1 | 7409 |
| ZAP70 | 7535 |

TC 49

| | |
|---|---|
| ACP5 | 54 |
| ADAM28 | 10863 |
| ADORA3 | 140 |
| APOC1 | 341 |
| APOL1 | 8542 |
| APOL6 | 80830 |
| ARRB2 | 409 |
| B2M | 567 |
| BST2 | 684 |
| C2 | 717 |
| CCL18 | 6362 |
| CD68 | 968 |
| CFLAR | 8837 |
| CHI3L1 | 1116 |
| CLEC5A | 23601 |
| CPVL | 54504 |
| CSTA | 1475 |
| CTSZ | 1522 |
| CXCL10 | 3627 |
| DAPP1 | 27071 |
| EMR2 | 30817 |
| FKBP15 | 23307 |
| FLVCR2 | 55640 |
| FTL | 2512 |
| GLUL | 2752 |
| GM2A | 2760 |
| GNA15 | 2769 |
| HCP5 | 10866 |
| HLA-A | 3105 |
| HMOX1 | 3162 |
| IFI35 | 3430 |
| IFI44L | 10964 |
| IFIT2 | 3433 |
| IFIT3 | 3437 |
| IFITM1 | 8519 |
| IGJ | 3512 |
| IGKC | 3514 |
| IGKV1OR15- | 339562 |
| 118 | |
| IGL@ | 3535 |
| IGLL3 | 91353 |
| IGLV2-23 | 28813 |
| IGSF6 | 10261 |
| IL15 | 3600 |
| IL15RA | 3601 |
| IRF7 | 3665 |
| ISG15 | 9636 |
| KMO | 8564 |
| LAMP3 | 27074 |
| LOC100130100 | 100130100 |
| LOC652493 | 652493 |
| MAN2B1 | 4125 |
| MAP3K8 | 1326 |
| MARCO | 8685 |
| MGAT1 | 4245 |
| MGAT4A | 11320 |
| MMP9 | 4318 |
| MX1 | 4599 |
| MX2 | 4600 |
| NAGK | 55577 |
| NFKBIA | 4792 |
| NFKBIE | 4794 |
| NINJ1 | 4814 |
| NR1H3 | 10062 |
| OAS2 | 4939 |
| OASL | 8638 |
| OLR1 | 4973 |
| PARP12 | 64761 |
| PARP8 | 79668 |
| PDE4B | 5142 |
| PLA2G7 | 7941 |
| PLEKHO1 | 51177 |
| PLTP | 5360 |
| RARRES1 | 5918 |
| RASGRP3 | 25780 |
| RASSF4 | 83937 |
| RHBDF2 | 79651 |
| RSAD2 | 91543 |
| RTP4 | 64108 |
| S100A8 | 6279 |
| S100A9 | 6280 |
| SAMD9 | 54809 |
| SECTM1 | 6398 |
| SIGLEC1 | 6614 |
| SLC1A3 | 6507 |
| SNX10 | 29887 |
| SPP1 | 6696 |
| STAT1 | 6772 |
| STK10 | 6793 |
| TAP1 | 6890 |
| TAP2 | 6891 |
| TCIRG1 | 10312 |
| TLR4 | 7099 |
| TLR7 | 51284 |
| TMEM140 | 55281 |
| TMEM176B | 28959 |
| TREM1 | 54210 |
| UBE2L6 | 9246 |
| WARS | 7453 |
| XAF1 | 54739 |

TC 50

| | |
|---|---|
| ADAP2 | 55803 |
| ALOX5 | 240 |
| ALOX5AP | 241 |
| APOE | 348 |
| APOL3 | 80833 |
| ARHGAP15 | 55843 |
| ARHGDIB | 397 |
| BCL2A1 | 597 |
| BIN2 | 51411 |
| BIRC3 | 330 |
| BTN3A3 | 10384 |
| C1ORF38 | 9473 |
| C1QA | 712 |
| C1QB | 713 |
| C5AR1 | 728 |
| CASP1 | 834 |
| CASP4 | 837 |
| CCL5 | 6352 |
| CD14 | 929 |
| CD163 | 9332 |
| CD2 | 914 |
| CD3D | 915 |
| CD4 | 920 |
| CD48 | 962 |
| CD52 | 1043 |
| CD69 | 969 |
| CD74 | 972 |
| CLEC2B | 9976 |
| CLEC4A | 50856 |
| CLIC2 | 1193 |
| CORO1A | 11151 |
| CTSB | 1508 |
| CTSC | 1075 |
| CUGBP2 | 10659 |
| CXCR4 | 7852 |
| CYSLTR1 | 10800 |
| CYTIP | 9595 |
| ENTPD1 | 953 |
| FAM49A | 81553 |
| FAS | 355 |
| FCER1G | 2207 |
| FCGR1A | 2209 |
| FCGR1B | 2210 |
| FCGR2A | 2212 |
| FCGR2B | 2213 |
| FCGR2C | 9103 |
| FCGR3A | 2214 |
| FCGR3B | 2215 |
| FGL2 | 10875 |
| FLI1 | 2313 |
| FOLR2 | 2350 |
| FYB | 2533 |
| GBP1 | 2633 |
| GBP2 | 2634 |
| GIMAP4 | 55303 |
| GIMAP5 | 55340 |
| GIMAP6 | 474344 |
| GPR183 | 1880 |
| HLA-B | 3106 |
| HLA-C | 3107 |
| HLA-DMB | 3109 |
| HLA-DPA1 | 3113 |
| HLA-DPB1 | 3115 |
| HLA-DQB1 | 3119 |
| HLA-DRA | 3122 |
| HLA-DRB1 | 3123 |
| HLA-E | 3133 |
| HLA-F | 3134 |
| HLA-G | 3135 |
| HMHA1 | 23526 |
| ICAM1 | 3383 |
| IFI16 | 3428 |
| IFI30 | 10437 |
| IL18BP | 10068 |
| IL2RG | 3561 |
| IL7R | 3575 |
| IRF1 | 3659 |
| IRF8 | 3394 |
| LAPTM5 | 7805 |
| LGALS9 | 3965 |
| LGMN | 5641 |
| LHFPL2 | 10184 |
| LIPA | 3988 |
| LOC648998 | 648998 |
| LPXN | 9404 |
| LY96 | 23643 |
| LYZ | 4069 |
| MAFB | 9935 |
| MRC1 | 4360 |
| MS4A4A | 51338 |
| MSR1 | 4481 |
| NAGA | 4668 |
| NCF2 | 4688 |
| NCKAP1L | 3071 |
| NPL | 80896 |
| PILRA | 29992 |
| PLEKHO2 | 80301 |
| PLXNC1 | 10154 |
| PRDM1 | 639 |
| PSMB10 | 5699 |
| PSMB9 | 5698 |
| PTPN22 | 26191 |
| PTPN6 | 5777 |
| RAC2 | 5880 |
| RARRES3 | 5920 |
| RGS1 | 5996 |
| RGS19 | 10287 |
| RHOG | 391 |
| RNASE6 | 6039 |
| SAMSN1 | 64092 |
| SASH3 | 54440 |
| SLC15A3 | 51296 |
| SLC31A2 | 1318 |
| SLC7A7 | 9056 |
| SLCO2B1 | 11309 |
| SP110 | 3431 |
| SRGN | 5552 |
| ST8SIA4 | 7903 |
| STK17B | 9262 |
| TBXAS1 | 6916 |
| TFEC | 22797 |
| TLR2 | 7097 |
| TM6SF1 | 53346 |
| TNFAIP3 | 7128 |
| TNFRSF1B | 7133 |
| TRAC | 28755 |
| TRBC1 | 28639 |
| TRBC2 | 28638 |
| TREM2 | 54209 |
| TRIM22 | 10346 |
| TYMP | 1890 |
| VAMP5 | 10791 |
| VSIG4 | 11326 |
| WIPF1 | 7456 |

TC 51

| | |
|---|---|
| ACSL5 | 51703 |
| AIM1 | 202 |
| AMPH | 273 |
| ANXA2 | 302 |
| ANXA2P2 | 304 |
| ANXA4 | 307 |
| ARPC1B | 10095 |
| BAI3 | 577 |
| BEX1 | 55859 |
| BHLHB9 | 80823 |
| BLNK | 29760 |
| CAND2 | 23066 |
| CAPG | 822 |
| CEBPB | 1051 |
| CLGN | 1047 |
| CLIC1 | 1192 |
| CRIP1 | 1396 |
| CTSH | 1512 |
| CXXC4 | 80319 |
| CYBA | 1535 |
| DENND2D | 79961 |
| ELOVL1 | 64834 |
| ELOVL2 | 54898 |
| FAM38A | 9780 |
| FGD1 | 2245 |
| FOSL2 | 2355 |
| FUCA1 | 2517 |
| GSTK1 | 373156 |
| HEXB | 3074 |
| IER3 | 8870 |
| IFI27 | 3429 |
| IL32 | 9235 |
| IL4R | 3566 |
| IPO9 | 55705 |
| ISG20 | 3669 |
| KCNH2 | 3757 |
| KIAA0746 | 23231 |
| KLF4 | 9314 |
| LGALS3 | 3958 |
| LRP10 | 26020 |
| LYN | 4067 |
| MAGED4B | 81557 |
| MAGEL2 | 54551 |
| MLLT11 | 10962 |
| MVP | 9961 |
| MYC | 4609 |
| NOVA1 | 4857 |
| NPC2 | 10577 |
| NUDT11 | 55190 |
| PARP4 | 143 |
| PCGF2 | 7703 |
| PDLIM1 | 9124 |
| PDZK1IP1 | 10158 |
| PEG3 | 5178 |
| PIP4K2B | 8396 |
| PLAUR | 5329 |
| PNMAL1 | 55228 |
| PPM1E | 22843 |
| PRR3 | 80742 |
| PSMB8 | 5696 |
| PTOV1 | 53635 |
| PYCARD | 29108 |
| RAB20 | 55647 |
| RBM47 | 54502 |
| RNASET2 | 8635 |
| RNFT2 | 84900 |
| S100A10 | 6281 |
| S100A11 | 6282 |
| S100A6 | 6277 |
| SALL2 | 6297 |
| SCO2 | 9997 |
| SDC4 | 6385 |
| SERPINB1 | 1992 |
| SH3BGRL3 | 83442 |
| SH3BP4 | 23677 |
| SLC22A17 | 51310 |
| SQRDL | 58472 |
| SV2A | 9900 |
| SYNGR2 | 9144 |
| TAGLN2 | 8407 |
| TM4SF1 | 4071 |
| TMBIM1 | 64114 |
| TMSB10 | 9168 |
| TMSB15A | 11013 |
| TNFSF13 | 8741 |
| TRO | 7216 |
| TSPO | 706 |
| UPP1 | 7378 |
| VAMP8 | 8673 |
| VDR | 7421 |
| ZFP36L2 | 678 |
| ZFP37 | 7539 |
| ZNF135 | 7694 |
| ZNF20 | 7568 |
| ZNF606 | 80095 |
| ZNF667 | 63934 |

Although the transcription clusters were identified by mathematical analysis, we have demonstrated that the transcription clusters have biological significance. We have found the transcription clusters to be highly enriched for a wide variety of basic biological structures or functions. Examples of associations between transcription clusters and basic biological structures or functions are listed in Table 2 below.

TABLE 2
Biological Structures and Functions Associated with Transcription Clusters

| Transcription | |
|---|---|
| Cluster No. | Associated Biological Structure and/or Function |
| 1 | Tumor Tissue-specific gene sets |
| 4 | Basiloid epithelial genes |
| 5 | Epithelial phenotype including desmosomal structure |
| 17 | RNA splicing |
| 22 | TGF-beta transcription |
| 26 | Proliferation |
| 27 | Cell cycle control |
| 29 | DNA integrity and regulation, nucleic-acid binding |
| 32 | Metabolism |
| 35 | Ribosomal proteins |
| 37 | vesicle and intracellular protein trafficking |
| 39 | Hypoxia responsive genes |
| 40 | Endothelial specific genes |
| 41 | Extracellular matrix, cell contact |
| 44 | Extracellular matrix genes |
| 45 | Extracellular matrix and cell communication |
| 46 | Endothelium and complement |
| 47 | Hematopoietic cells: CD8 Tcell enriched |
| 48 | Hematopoietic cells Bcell Tcell NK cell enriched |
| 49 | Hematopoietic cells dendritic cell, monocyte enriched |
| 50 | Myeloid cells |

For some transcription clusters, the associated biology (structure and/or function), is presumed to exist, but has not been identified yet. It is important to note, however, that the practice of the methods disclosed herein, e.g., identifying a PGS for classifying a cancerous tissue as sensitive or resistant to an anticancer drug, does not require knowledge of any biological structure or function associated with any transcription cluster. Utilization of the methods described herein depends solely on two types of correlations: (1) the correlations among transcript levels within each transcription cluster; and (2) the correlation between the mean expression score for a transcription cluster and phenotype, e.g., drug sensitivity versus drug resistance, or good prognosis versus poor prognosis. Our discovery that many different basic biological structures and functions are associated with, or represented by, the disclosed transcription clusters, is strong evidence that numerous and varied phenotypic traits can be correlated readily with one or more of the transcription clusters by a person of skill in the art, without undue experimentation.

Once a transcription cluster has been associated with a phenotype of interest (such as tumor sensitivity or resistance to a particular drug), that transcription cluster (or a subset of that transcription cluster) can be used as a multigene biomarker for that phenotype. In other words, a transcription cluster, or a subset thereof, is a PGS for the phenotype(s) associated with that transcription cluster. Any given transcription cluster can be associated with more than one phenotype.

A phenotype can be associated with more than one transcription cluster. The more than one transcription cluster, or subsets thereof, can be a PGS for the phenotype(s) associated with those transcription clusters.

In certain embodiments, one or more transcription clusters from Table 1 may be optionally excluded from the analysis. For example, TC1, TC2, TC3, TC4, TC5, TC6, TC7, TC8, TC9, TC10, TC11, TC12, TC13, TC14, TC15, TC16, TC17, TC18, TC19, TC20, TC21, TC22, TC23, TC24, TC25, TC26, TC27, TC28, TC29, TC30, TC31, TC32, TC33, TC34, TC35, TC36, TC37, TC38, TC39, TC40, TC41, TC42, TC43, TC44, TC45, TC46, TC47, TC48, TC49, TC50, or TC51 may be excluded from the analysis.

In order to practice the methods disclosed herein, the skilled person needs gene expression data, e.g., conventional microarray data or quantitative PCR data, from: (a) a population shown to be positive for the phenotype of interest, and (b) a population shown to be negative for the phenotype of interest (collectively, “response data”). Examples of populations that can be used to generate response data include populations of tissue samples (tumor samples or blood samples) that represent populations of human patients or animal models, for example, mouse models of cancer. The necessary response data can be obtained readily by the skilled person, using nothing more than conventional methods, materials and instrumentation for measuring gene expression or transcript abundance in a tissue sample. Suitable methods, materials and instrumentation are well-known and commercially available. Once the response data are in hand, the methods described herein can be performed by using the lists of genes in the transcription clusters set forth above in Table 1, and mathematical calculations that are described herein.

As described in more detail in Example 2 below, we measured the transcript levels of subsets of genes from all 51 transcription clusters in tissue samples from a population of tumor samples shown to be sensitive to tivozanib; and a population of tumor samples shown to be resistant to tivozanib. Next, we calculated a cluster score for each cluster, in each individual in each population. Then, with respect to each transcription cluster, we used a Student's t-test to calculate whether the cluster scores of the tivozanib-sensitive population was significantly different from the cluster scores of the tivozanib-resistant population. We found that with regard to TC50, there was a statistically significant difference between the cluster scores of the tivozanib-sensitive population and the cluster scores of the tivozanib-resistant population.

The transcription clusters disclosed herein resulted from a genome-wide analysis, and the transcription clusters represent widely divergent biological structures and functions that are not unique to cancer biology. The transcription cluster useful for predicting response to tivozanib, TC50, is highly enriched for genes expressed by a particular class of hematopoietic cells that infiltrate certain tumors. Hematopoietic cells are critical for many biological processes. In principle, any phenotype mediated by this class of hematopoietic cells can be identified by a test for expression of TC50.

### Phenotypically-Defined Populations

Populations.

The methods disclosed herein can be used on the basis of: (a) gene expression data (transcript abundance data) from a population of human patients, animal models or tumors, shown to be positive for the phenotypic trait of interest, e.g., response to a particular drug, or cancer prognosis; together with (b) relative gene expression data or relative transcript abundance data from populations shown to differ with respect to a phenotypic trait of interest, such as sensitivity to a particular cancer drug, and/or overall prognosis in cancer treatment. Preferably, the classified populations that differ in the phenotypic trait of interest are otherwise generally comparable. For example, if a drug sensitive population is a group of a particular strain of mice, the resistant population should be a group of the same strain of mice. In another example, if the sensitive population is a set of human kidney tumor biopsy samples, the resistant population should be a set of human kidney tumor biopsy samples.

Phenotype Definition.

Suitable criteria for phenotypic classification will depend on the phenotypes of interest. For example, if the phenotypes of interest are sensitivity and resistance of tumors to treatment with a particular anti-tumor agent, tumors can be classified on the basis of one or more parameters such as tumor growth inhibition (TGI) assessed at a single endpoint, TGI assessed over time in terms of a growth curve, or tumor histology. For a given parameter, a threshold or cut-off value can be set for distinguishing a positive phenotype from a negative phenotype. A particular percent TGI is sometimes used as a threshold or cut-off. For example, this could be clinically defined RECIST criteria (Response Evaluation Criteria In Solid Tumors) for measuring TGI in human clinical trials. In another example, the timing of an inflection point in a tumor growth curve is used. In another example, a given score in a histological assessment is used. There is considerable latitude in selection of suitable parameters and suitable thresholds for phenotype definition. For anti-tumor drug response classification, suitable phenotype definitions will depend on factors including the tumor type and the particular drug involved. Selection of suitable parameters and suitable thresholds for phenotype definition are within skill in the art.

### Gene Expression Data

Tissue Samples.

A tissue sample from a tumor in a human patient or a tumor in mouse model can be used as a source of RNA, so that an individual mean expression score for each transcription cluster, and a population mean expression score for each transcription cluster, can be determined. Examples of tumors are carcinomas, sarcomas, gliomas and lymphomas. The tissue sample can be obtained by using conventional tumor biopsy instruments and procedures. Endoscopic biopsy, excisional biopsy, incisional biopsy, fine needle biopsy, punch biopsy, shave biopsy and skin biopsy are examples of recognized medical procedures that can be used by one of skill in the art to obtain tumor samples for use in practicing the invention. The tumor tissue sample should be large enough to provide sufficient RNA for measuring individual gene expression levels.

The tumor tissue sample can be in any form that allows quantitative analysis of gene expression or transcript abundance. In some embodiments, RNA is isolated from the tissue sample prior to quantitative analysis. Some methods of RNA analysis, however, do not require RNA extraction, e.g., the gNPA™ technology commercially available from High Throughput Genomics, Inc. (Tucson, Ariz.). Accordingly, the tissue sample can be fresh, preserved through suitable cryogenic techniques, or preserved through non-cryogenic techniques. Tissue samples used in the invention can be clinical biopsy specimens, which often are fixed in formalin and then embedded in paraffin. Samples in this form are commonly known as formalin-fixed, paraffin-embedded (FFPE) tissue. Techniques of tissue preparation and tissue preservation suitable for use in the present invention are well-known to those skilled in the art.

Expression levels for a representative number of genes from a given transcription cluster are the input values used to calculate the individual mean expression score for that transcription cluster, in a given tissue sample. Each tissue sample is a member of a population, e.g., a sensitive population or a resistant population. The individual mean expression scores for all the individuals in a given population then are used to calculate the population mean expression score for a given transcription cluster, in a given population. So for each tissue sample, it is necessary to determine, i.e., measure, the expression levels of individual genes in a transcription cluster. Gene expression levels (transcript abundance) can be determined by any suitable method. Exemplary methods for measuring individual gene expression levels include DNA microarray analysis, qRT-PCR, gNPA™, the NanoString® technology, and the QuantiGene® Plex assay system, each of which is discussed below.

RNA Isolation.

DNA microarray analysis and qRT-PCR generally involve RNA isolation from a tissue sample. Methods for rapid and efficient extraction of eukaryotic mRNA, i.e., poly(a) RNA, from tissue samples are well-established and known to those of skill in the art. See, e.g., Ausubel et al., 1997, Current Protocols of Molecular Biology, John Wiley & Sons. The tissue sample can be fresh, frozen or fixed paraffin-embedded (FFPE) clinical study tumor specimens. In general, RNA isolated from fresh or frozen tissue samples tends to be less fragmented than RNA from FFPE samples. FFPE samples of tumor material, however, are more readily available, and FFPE samples are suitable sources of RNA for use in methods of the present invention. For a discussion of FFPE samples as sources of RNA for gene expression profiling by RT-PCR, see, e.g., Clark-Langone et al., 2007, BMC Genomics 8:279. Also see, De Andrés et al., 1995, Biotechniques 18:42044; and Baker et al., U.S. Patent Application Publication No. 2005/0095634. The use of commercially available kits with vendor's instructions for RNA extraction and preparation is widespread and common. Commercial vendors of various RNA isolation products and complete kits include Qiagen (Valencia, Calif.), Invitrogen (Carlsbad, Calif.), Ambion (Austin, Tex.) and Exiqon (Woburn, Mass.).

In general, RNA isolation begins with tissue/cell disruption. During tissue/cell disruption, it is desirable to minimize RNA degradation by RNases. One approach to limiting RNase activity during the RNA isolation process is to ensure that a denaturant is in contact with cellular contents as soon as the cells are disrupted. Another common practice is to include one or more proteases in the RNA isolation process. Optionally, fresh tissue samples are immersed in an RNA stabilization solution, at room temperature, as soon as they are collected. The stabilization solution rapidly permeates the cells, stabilizing the RNA for storage at 4° C., for subsequent isolation. One such stabilization solution is available commercially as RNAlater® (Ambion, Austin, Tex.).

In some protocols, total RNA is isolated from disrupted tumor material by cesium chloride density gradient centrifugation. In general, mRNA makes up approximately 1% to 5% of total cellular RNA. Immobilized oligo(dT), e.g., oligo(dT) cellulose, is commonly used to separate mRNA from ribosomal RNA and transfer RNA. If stored after isolation, RNA must be stored under RNase-free conditions. Methods for stable storage of isolated RNA are known in the art. Various commercial products for stable storage of RNA are available.

Microarray Analysis.

The mRNA expression level for multiple genes can be measured using conventional DNA microarray expression profiling technology. A DNA microarray is a collection of specific DNA segments or probes affixed to a solid surface or substrate such as glass, plastic or silicon, with each specific DNA segment occupying a known location in the array. Hybridization with a sample of labeled RNA, usually under stringent hybridization conditions, allows detection and quantitation of RNA molecules corresponding to each probe in the array. After stringent washing to remove non-specifically bound sample material, the microarray is scanned by confocal laser microscopy or other suitable detection method. Modern commercial DNA microarrays, often known as DNA chips, typically contain tens of thousands of probes, and thus can measure expression of tens of thousands of genes simultaneously. Such microarrays can be used in practicing the disclosed methods. Alternatively, custom chips containing as few probes as those needed to measure expression of the genes of the transcription clusters, plus any desired controls or standards.

To facilitate data normalization, a two-color microarray reader can be used. In a two-color (two-channel) system, samples are labeled with a first fluorophore that emits at a first wavelength, while an RNA or cDNA standard is labeled with a second fluorophore that emits at a different wavelength. For example, Cy3 (570 nm) and Cy5 (670 nm) often are employed together in two-color microarray systems.

DNA microarray technology is well-developed, commercially available, and widely employed. Therefore, in performing the methods disclosed herein, the skilled person can use microarray technology to measure expression levels of genes in the transcription cluster without undue experimentation. DNA microarray chips, reagents (such as those for RNA or cDNA preparation, RNA or cDNA labeling, hybridization and washing solutions), instruments (such as microarray readers) and protocols are well-known in the art and available from various commercial sources. Commercial vendors of microarray systems include Agilent Technologies (Santa Clara, Calif.) and Affymetrix (Santa Clara, Calif.), but other microarray systems can be used.

Quantitative RT-PCR.

The level of mRNA representing individual genes in a transcription cluster can be measured using conventional quantitative reverse transcriptase polymerase chain reaction (qRT-PCR) technology. Advantages of qRT-PCR include sensitivity, flexibility, quantitative accuracy, and ability to discriminate between closely related mRNAs. Guidance concerning the processing of tissue samples for quantitative PCR is available from various sources, including manufacturers and vendors of commercial products for qRT-PCR (e.g., Qiagen (Valencia, Calif.) and Ambion (Austin, Tex.)). Instrument systems for automated performance of qRT-PCR are commercially available and used routinely in many laboratories. An example of a well-known commercial system is the Applied Biosystems 7900HT Fast Real-Time PCR System (Applied Biosystems, Foster City, Calif.).

Once isolated mRNA is in hand, the first step in gene expression profiling by RT-PCR is the reverse transcription of the mRNA template into cDNA, which is then exponentially amplified in a PCR reaction. Two commonly used reverse transcriptases are avilo myeloblastosis virus reverse transcriptase (AMV-RT) and Moloney murine leukemia virus reverse transcriptase (MMLV-RT). The reverse transcription reaction typically is primed with specific primers, random hexamers, or oligo(dT) primers. Suitable primers are commercially available, e.g., GeneAmp® RNA PCR kit (Perkin Elmer, Waltham, Mass.). The resulting cDNA product can be used as a template in the subsequent polymerase chain reaction.

The PCR step is carried out using a thermostable DNA-dependent DNA polymerase. The polymerase most commonly used in PCR systems is a Thermus aquaticus (Taq) polymerase. The selectivity of PCR results from the use of primers that are complementary to the DNA region targeted for amplification, i.e., regions of the cDNAs reverse transcribed from the genes of the Transcription Cluster. Therefore, when qRT-PCR is employed in the present invention, primers specific to each gene in a given Transcription Cluster are based on the cDNA sequence of the gene. Commercial technologies such as SYBR® green or TaqMan® (Applied Biosystems, Foster City, Calif.) can be used in accordance with the vendor's instructions. Messenger RNA levels can be normalized for differences in loading among samples by comparing the levels of housekeeping genes such as beta-actin or GAPDH. The level of mRNA expression can be expressed relative to any single control sample such as mRNA from normal, non-tumor tissue or cells. Alternatively, it can be expressed relative to mRNA from a pool of tumor samples, or tumor cell lines, or from a commercially available set of control mRNA.

Suitable primer sets for PCR analysis of expression levels of genes in a transcription cluster can be designed and synthesized by one of skill in the art, without undue experimentation. Alternatively, complete PCR primer sets for practicing the disclosed methods can be purchased from commercial sources, e.g., Applied Biosystems, based on the identities of genes in the transcription clusters, as listed in Table 1. PCR primers preferably are about 17 to 25 nucleotides in length. Primers can be designed to have a particular melting temperature (Tm), using conventional algorithms for Tm estimation. Software for primer design and Tm estimation are available commercially, e.g., Primer Express™ (Applied Biosystems), and also are available on the internet, e.g., Primer3 (Massachusetts Institute of Technology). By applying established principles of PCR primer design, a large number of different primers can be used to measure the expression level of any given gene. Accordingly, the disclosed methods are not limited with respect to which particular primers are used for any given gene in a transcription cluster.

Quantitative Nuclease Protection Assay.

An example of a suitable method for determining expression levels of genes in a transcription cluster without performing an RNA extraction step is the quantitative nuclease protection assay (qNPAT™), which is commercially available from High Throughput Genomics, Inc. (aka “HTG”; Tucson, Ariz.). In the qNPA method, samples are treated in a 96-well plate with a proprietary Lysis Buffer (HTG), which releases total RNA into solution. Gene-specific DNA oligonucleotides, i.e., specific for each gene in a given Transcription Cluster, are added directly to the Lysis Buffer solution, and they hybridize to the RNA present in the Lysis Buffer solution. The DNA oligonucleotides are added in excess, to ensure that all RNA molecules complementary to the DNA oligonucleotides are hybridized. After the hybridization step, S1 nuclease is added to the mixture. The S1 nuclease digests the non-hybridized portion of the target RNA, all of the non-target RNA, and excess DNA oligonucleotides. Then the S1 nuclease enzyme is inactivated. The RNA::DNA heteroduplexes are treated to remove the RNA portion of the duplex, leaving only the previously protected oligonucleotide probes. The surviving DNA oligonucleotides are a stoichiometrically representative library of the original RNA sample. The qNPA oligonucleotide library can be quantified using the ArrayPlate Detection System (HTG).

NanoString® nCounter® Analysis.

Another example of a technology suitable for determining expression levels of genes in a transcription cluster is a commercially available assay system based on probes with molecular “barcodes” is the NanoString® nCounter™ Analysis system (NanoString® Technologies, Seattle, Wash.). This system is designed to detect and count hundreds of unique transcripts in a single reaction. Each color-coded barcode is attached to a single target-specific probe corresponding to a gene interest, e.g., a gene in a transcription cluster. When mixed together with controls, probes form a multiplexed “CodeSet.” The NanoString® technology employs two approximately 50-base probes per mRNA, that hybridize in solution. A “reporter probe” carries the signal, and a “capture probe” allows the complex to be immobilized for data collection. After hybridization, the excess probes are removed, and the probe/target complexes are aligned and immobilized in nCounter® cartridges, which are placed in a digital analyzer. The nCounter® analysis system is an integrated system comprising an automated sample prep station, a digital analyzer, the CodeSet (molecular barcodes), and all of the reagents and consumables needed to perform the analysis.

QuantiGene® Plex Assay.

Another example of a technology suitable for determining expression levels of genes in a transcription cluster is a commercially available assay system known as the QuantiGene® Plex Assay (Panomics, Fremont, Calif.). This technology combines branched DNA signal amplification with xMAP (multi-analyte profiling) beads, to enable simultaneous quantification of multiple RNA targets directly from fresh, frozen or FFPE tissue samples, or purified RNA preparations. For further description of this technology, see, e.g., Flagella et al., 2006, Anal. Biochem. 352:50-60.

Practice of the methods disclosed herein is not limited to the use of any particular technology for generation of gene expression data. As discussed above, various accurate and reliable systems, including protocols, reagents and instrumentation are commercially available. Selection and use of a suitable system for generating gene expression data for use in the methods described herein is a design choice, and can be accomplished by a person of skill in the art, without undue experimentation.

Cluster Scores and Statistical Differences between Populations

A cluster score for any given transcription cluster in each tissue sample can be calculated according to the following algorithm:

$$cluster . score = \frac{1}{n} * {\sum }_{i = 1}^{n} Ei$$

wherein E1, E2, . . . En are the relative expression values obtained with respect to each of the n genes representing each transcription cluster.

A cluster score can be calculated for each of the 51 transcription clusters in each tissue sample in the drug sensitive population and each member tissue sample in the drug resistant population.

Statistical significance can be calculated in various ways well-known in the art, e.g., a t-test or a Kolmogorov-Smirnov test. For example, a Student's t-test can be performed by using the cluster score of each individual and then calculating a p-value using a two sample t-test between the drug sensitive population and the drug resistant population. See Example 2 below. Another suitable method is to do a Kolmogorov-Smirnov test as in the GSEA algorithm described in Subramanian, Tamayo et al., 2005, Proc. Nat'l Acad. Sci USA 102:15545-15550). Statistical significance may also be calculated by applying Fisher's exact test (Fisher, 1922, J. Royal Statistical Soc. 85:87-94; Agresti, 1992, Statistical Science 7:131-153) to calculate p-value between the drug sensitive population and the drug resistant population.

A statistically significant difference may be based on commonly used statistical cutoffs well-known in the art. For example, a statistically significant difference may be a p-value of less than or equal to 0.05, 0.01, 0.005, 0.001. The p-value can be calculated using algorithms such as the Student's t-test, the Kolmogorov-Smimov test, or the Fisher's exact test. It is contemplated herein that determining a statistically significant difference, using a suitable algorithm, is within the skill in the art, and that the skilled person can select an appropriate statistical cutoff for determining significance, based on the drug and population (e.g., tumor sample or patient population) being tested.

### Subsets of Transcription Clusters

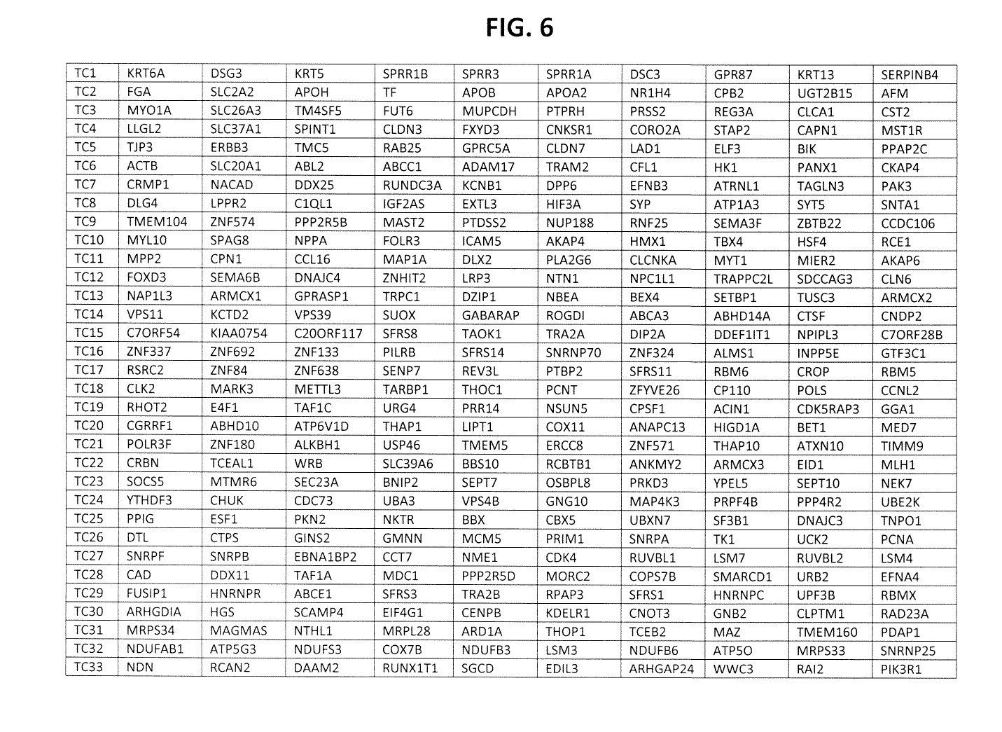

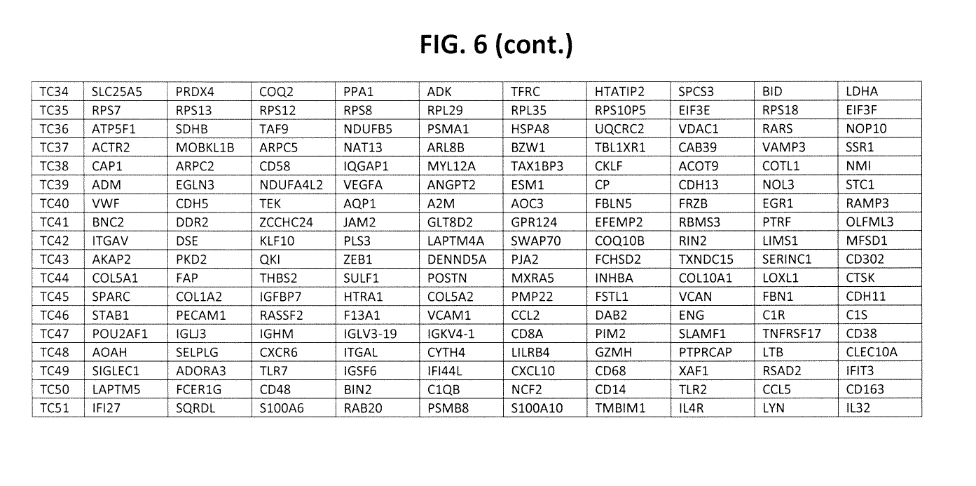

In some embodiments, the correlation between expression of a transcription cluster and a phenotype of interest, e.g., drug resistance, is established through the use of expression measurements for all the genes in a transcription cluster. However, the use of expression measurements for all the genes in a transcription cluster is optional. In some embodiments, the correlation between expression of a transcription cluster and a phenotype is established through the use of expression measurements for a subset, i.e., a representative number of genes, from the transcription cluster. Subsets of a transcription cluster can be used reliably to represent the entire transcription cluster, because within each transcription cluster, the genes are expressed coherently. By definition, gene expression levels (as represented by transcript abundance) within a given transcription cluster are correlated. In general, a larger subset generally yields a more accurate cluster score, with the marginal increase in accuracy per additional gene decreasing, as the size of the subset increases. A smaller subset provides convenience and economy. For example, if each transcription cluster is represented by 10 genes, the entire set of 51 transcription clusters can be effectively represented by only 510 probes, which can be incorporated into a single microarray chip, a single PCR kit, a single nCounter Analysis™ assay (NanoString® Technologies), or a single QuantiGene® Plex assay (Panomics, Fremont, Calif.), using technology that is currently available from commercial vendors. FIG. 6 lists 510 human genes, wherein each of the 51 transcription clusters is represented by a subset of only 10 genes.

Such a reduction in the number of probes can be advantageous in biomarker discovery projects, i.e., associating clinical phenotypes in oncology (drug response or prognosis) with specific sets of biologically relevant genes (biomarkers), and in clinical assays. Often, in clinical practice, small amounts of tissue are collected, without regard to preserving the integrity of the RNA in the sample. Consequently, the quantity and quality of RNA can be insufficient for precise measurement of the expression of large numbers of genes. By greatly reducing the number of genes to be assayed, e.g., a 100-fold reduction, the use of subsets of the transcription clusters enables robust transcription cluster analysis from small tissue amounts, yielding low quality RNA.

The optimal number of genes employed to represent each transcription cluster can be viewed as a balance between assay robustness and convenience. When a subset of a transcription cluster is used, the subset preferably contains ten or more genes. The selection of a suitable number to be the representative number can be done by a person of skill in the art, without undue experimentation.

We sought to demonstrate with mathematical rigor, that essentially any subset of at least ten genes from any one of Transcription Clusters 1-51 would be a highly effective surrogate for the entire transcription cluster from which it was taken. In other words, we sought to determine whether any randomly selected 10-gene subset would yield an individual mean expression score highly correlated with the individual mean expression score calculated from expression scores for every member of the respective transcription cluster. To accomplish this, we generated 10,000 randomly chosen 10-gene subsets from each transcription cluster. Then we calculated the correlation between each of the 10,000 individual mean expression scores and the individual mean expression score for all genes of the transcription cluster.

Table 3 shows the worst correlation p-value of the 10,000 Pearson correlation comparisons for every transcription cluster. For each of the 51 transcription clusters, every one of the 10,000 randomly selected 10-gene subsets yields an individual mean expression score that is significantly correlated with the individual mean expression score calculated from the complete transcription cluster. This is a rigorous mathematical demonstration that essentially any 10-gene subset from any of the 51 transcription clusters is sufficiently representative of the entire transcription cluster, that it can be employed as a highly effective surrogate for the entire transcription cluster, thereby greatly reducing the number of gene expression measurements (and thus, the number of probes) needed to establish an association between a transcription cluster and a phenotype of interest.

TABLE 3
Worst p-Values from 10,000 Randomly-Chosen
Subsetsfor each Transcription Cluster

| TC No. | p-value |
|---|---|
| 01 | 0 |
| 02 | 0 |
| 03 | 0 |
| 04 | 6.40E−99 |
| 05 | 0 |
| 06 | 7.81E−129 |
| 07 | 1.29E−129 |
| 08 | 2.19E−223 |
| 09 | 3.89E−202 |
| 10 | 3.71E−09 |
| 11 | 6.91E−210 |
| 12 | 2.05E−189 |
| 13 | 2.34E−177 |
| 14 | 6.38E−132 |
| 15 | 0 |
| 16 | 2.01E−150 |
| 17 | 0 |
| 18 | 0 |
| 19 | 0 |
| 20 | 8.61E−219 |
| 21 | 4.50E−161 |
| 22 | 5.68E−194 |
| 23 | 1.55E−153 |
| 24 | 1.60E−188 |
| 25 | 0 |
| 26 | 0 |
| 27 | 0 |
| 28 | 1.57E−67 |
| 29 | 3.84E−219 |
| 30 | 0 |
| 31 | 1.60E−133 |
| 32 | 0 |
| 33 | 3.61E−124 |
| 34 | 1.74E−163 |
| 35 | 0 |
| 36 | 1.34E−206 |
| 37 | 3.04E−207 |
| 38 | 1.20E−143 |
| 39 | 0 |
| 40 | 0 |
| 41 | 0 |
| 42 | 1.58E−132 |
| 43 | 4.80E−228 |
| 44 | 0 |
| 45 | 0 |
| 46 | 0 |
| 47 | 0 |
| 48 | 0 |
| 49 | 0 |
| 50 | 0 |
| 51 | 1.86E−127 |

In Table 3, 0 denotes a p-value less than 5.40E−267.

In a further example of subset-based embodiments, we demonstrated with mathematical rigor that, for any of the transcription clusters, any ten-gene subset comprising at least five genes from the subset representing that cluster in FIG. 6, and at most five different genes randomly chosen from the transcription cluster in question, yields an individual mean expression score that is significantly correlated with the individual mean expression score calculated from expression scores for every member of that transcription cluster. In other words, for each of the 51 transcription clusters represented in FIG. 6, up to five genes in the ten-gene subset can be substituted with different genes chosen from the same transcription cluster in Table 1.

In this demonstration, for each of the 51 transcription clusters, we generated 10,000 new ten-gene subsets wherein at least five genes were taken from the ten-gene subset representing that cluster in FIG. 6, and at most five additional genes were chosen randomly from the cluster. Then we calculated the correlation between each of the 10,000 individual mean expression scores and the individual mean expression score for all genes of the transcription cluster. The worst correlation p-values of the 10,000 Pearson correlation comparisons for TC1-25, TC27-36 and TC38-51 were less than 5.40E-267. The worst correlation p-value of the 10,000 Pearson correlation comparisons for TC26 was 3.7E-126 and for TC37 was 2.3E-128. For each of the 51 transcription clusters, every one of the 10,000 new 10-gene subsets yields an individual mean expression score that is significantly correlated with the individual mean expression score calculated from the complete transcription cluster. This is a rigorous mathematical demonstration that essentially any 10-gene subset containing at least five genes from a 10-gene example in FIG. 6 and up to five randomly chosen genes from the same transcription cluster is sufficiently representative of the entire transcription cluster, so that it can be employed as a highly effective surrogate for the entire transcription cluster. This is advantageous, because it greatly reduces the number of gene expression measurements (and thus, the number of probes) needed to establish an association between a transcription cluster and a phenotype of interest. One of skill in the art will recognize that this is an example within the broader demonstration above (Table 3 and associated discussion) that essentially any ten-gene subset from any transcription cluster in Table 1 can be used as a surrogate for the entire transcription cluster.

### Predictive Gene Set (PGS)

A predictive gene set (PGS) is a multigene biomarker that is useful for classifying a type of tissue, e.g., a mammalian tumor, with respect to a particular phenotype. Examples of particular phenotypes are: (a) sensitive to a particular cancer drug; (b) resistant to a particular cancer drug; (c) likely to have a good outcome upon treatment (good prognosis); and (d) likely to have a poor outcome upon treatment (poor prognosis).

Disclosed herein is a general method for identifying novel predictive gene sets by using one or more of the 51 transcription clusters set forth herein. When a transcription cluster is shown to yield cluster scores significantly correlated with a phenotype of interest, the PGS is based on, or derived from, that transcription cluster. In some embodiments, the PGS includes all the genes in the transcription cluster. In other embodiments, the PGS includes only a subset of genes from the transcription cluster, rather than the entire transcription cluster. Preferably, a PGS identified using the methods described herein will include ten or more genes, e.g., 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 44, 46, 48 or 50 genes from the transcription cluster.

In some embodiments, more than one transcription cluster is associated with a phenotype of interest. In such a situation, a PGS can be based on any one of the associated transcription clusters, or a multiplicity of the associated transcription clusters.

### PGS Score

The predictive value of a PGS is achieved by measuring (with respect to a tissue sample) the expression levels of each of at least 10 of the genes in the PGS, and calculating a PGS score for the tissue sample according to the following algorithm:

$$P G S . score = \frac{1}{n} * {\sum }_{i = 1}^{n} Ei$$

wherein E1, E2, . . . En are the expression values of the n genes in the PGS.

Optionally, expression levels of additional genes, e.g., housekeeping genes to be used as internal standards, may be measured in addition to the PGS.

It should be noted that although the algorithms for calculating cluster scores and PGS scores are essentially the same, and both calculations involve gene expression values, a cluster score is not the same as a PGS score. The difference is in the context. A cluster score is associated with a sample of known phenotype, which sample is being used in a method of identifying a PGS. In contrast, a PGS score is associated with a sample of unknown phenotype, which sample is being tested and classified as to likely phenotype.

### PGS Score Interpretation

PGS scores are interpreted with respect to a threshold PGS score. PGS scores higher than the threshold PGS score will be interpreted as indicating a tissue sample classified as likely to have a first phenotype, e.g., a tumor likely to be sensitive to treatment a particular drug. PGS scores lower than the threshold PGS score will be interpreted as indicating a tissue sample classified as likely to have a second phenotype, e.g., a tumor likely to be resistant to treatment with the drug. With respect to tumors, a given threshold PGS score may vary, depending on tumor type. In the context of the disclosed methods, the term “tumor type” takes into account (a) species (mouse or human); and (b) organ or tissue of origin. Optionally, tumor type further takes into account tumor categorization based on gene expression characteristics, e.g., HER2-positive breast tumors, or non-small cell lung tumors expressing a particular EGFR mutation.

For any given tumor type, an optimum threshold PGS score can be determined (or at least approximated) empirically by performing a threshold determination analysis. Preferably, threshold determination analysis includes receiver operator characteristic (ROC) curve analysis.

ROC curve analysis is a well-known statistical technique, the application of which is within ordinary skill in the art. For a discussion of ROC curve analysis, see generally Zweig et al., 1993, “Receiver operating characteristic (ROC) plots: a fundamental evaluation tool in clinical medicine,” Clin. Chem. 39:561-577; and Pepe, 2003, The statistical evaluation of medical tests for classification and prediction, Oxford Press, New York.

PGS scores and the optimum threshold PGS score may vary from tumor type to tumor type. Therefore, a threshold determination analysis preferably is performed on one or more datasets representing any given tumor type to be tested using the disclosed methods. The dataset used for threshold determination analysis includes: (a) actual response data (response or non-response), and (b) a PGS score for each tumor sample from a group of human tumors or mouse tumors. Once a PGS score threshold is determined with respect to a given tumor type, that threshold can be applied to interpret PGS scores from tumors of that tumor type.

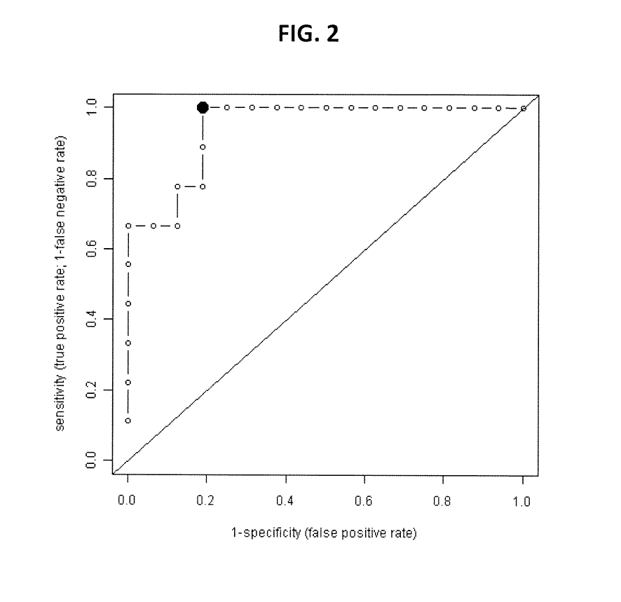

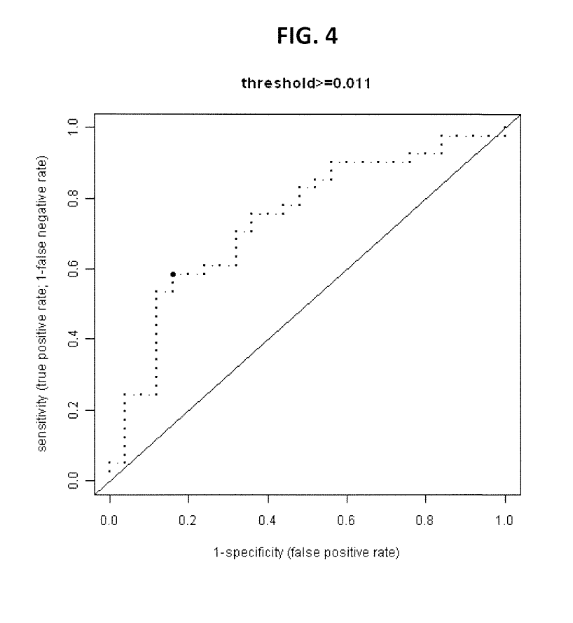

The ROC curve analysis is performed essentially as follows. Any sample with a PGS score greater than threshold is identified as a non-responder. Any sample with a PGS score less than or equal to threshold is identified as responder. For every PGS score from a tested set of samples, “responders” and “non-responders” (hypothetical calls) are classified using that PGS score as the threshold. This process enables calculation of TPR (y vector) and FPR (x vector) for each potential threshold, through comparison of hypothetical calls against the actual response data for the data set. Then an ROC curve is constructed by making a dot plot, using the TPR vector, and FPR vector. If the ROC curve is above the diagonal from (0, 0) point to (1.0, 1.0) point, it shows that the PGS test result is a better test than random (see, e.g., FIGS. 2 and 4).

The ROC curve can be used to identify the best operating point. The best operating point is the one that yields the best balance between the cost of false positives weighed against the cost of false negatives. These costs need not be equal. The average expected cost of classification at point x,y in the ROC space is denoted by the expression

C=(1−p)alpha*x+p*beta(1−y)

wherein:

alpha=cost of a false positive,

beta=cost of missing a positive (false negative), and

p=proportion of positive cases.

False positives and false negatives can be weighted differently by assigning different values for alpha and beta. For example, if the phenotypic trait of interest is drug response, and it is decided to include more patients in the responder group at the cost of treating more patients who are non-responders, one can put more weight on alpha. In this case, it is assumed that the cost of false positive and false negative is the same (alpha equals to beta). Therefore, the average expected cost of classification at point x,y in the ROC space is:

C′=(1−p)*x+p*(1−y).

The smallest C′ can be calculated after using all pairs of false positive and false negative (x, y). The optimum PGS score threshold is calculated as the PGS score of the (x, y) at C′. For example, as shown in Example 2, the optimum PGS score threshold, as determined using this approach, was found to be 1.62.

In addition to predicting whether a tumor will be sensitive or resistant to treatment with a particular drug, e.g., tivozanib, a PGS score provides an approximate, but useful, indication of how likely a tumor is to be sensitive or resistant, according to the magnitude of the PGS score.

### EXAMPLES

The invention is further illustrated by the following examples. The examples are provided for illustrative purposes only, and are not to be construed as limiting the scope or content of the invention in any way.

### Example 1

### Murine Tumors—BH Archive

A genetically diverse population of more than 100 murine breast tumors (BH archive) was used to identify tumors that are sensitive to a drug of interest (responders) and tumors that are resistant to the same drug of interest (non-responders). The BH archive was established by in vivo propagation and cryopreservation of primary tumor material from more than 100 spontaneous murine breast tumors derived from engineered chimeric mice that develop HER2-dependent, inducible spontaneous breast tumors.

The mice were produced essentially as follows. Ink4a homozygous null murine ES cells were co-transfected with the following four constructs, as separate fragments: MMTV-rtTA, TetO-HER2^{V659Eneu}, TetO-luciferase and PGK-puromycin. ES cells carrying these constructs were injected into 3-day-old C57BL/6 blastocysts, which were transplanted into pseudo-pregnant female mice for gestation leading to birth of the chimeric mice. The mouse mammary tumor virus long terminal repeat (MMTV) was used to drive breast-specific expression of the reverse tetracycline transactivator (rtTA). The rtTA provided for breast-specific expression of the HER2 activated oncogene, when doxycycline was provided to the mice in their drinking water. Following induction of the tetracycline-responsive promoter by doxycycline, the mice developed invasive mammary carcinomas with a latency of about 2 to 6 months.

The BH archive of more than 100 tumors was produced essentially as follows. Primary tumor cells were isolated from the chimeric animals by physical disruption of the tumors using cell strainers. Typically 1×105 cells were mixed with Matrigel (50:50 by vol.) and injected subcutaneously into female NCr nu/nu mice. When these tumors grew to approximately 500 mm3, which typically required 2 to 4 weeks, they were collected for one further round of in vivo propagation, after which tumor material was cryopreserved in liquid nitrogen. To characterize the propagated and archived tumors, 1×105 cells from each individual tumor line were thawed and injected subcutaneously in BALB/c nude mice. When the tumors reached a mean size of 500 to 800 mm3, animals were sacrificed and tumors were surgically removed for further analysis.

The BH tumor archive was characterized at the tissue, cellular and molecular level. Analyses included general histopathology (architecture, cytology, desmoplasia, extent of necrosis, vasculature morphology), IHC (e.g., CD31 for tumor vasculature, Ki67 for tumor cell proliferation, signaling proteins for pathway activation), and global molecular profiling (microarray for RNA expression, array CGH for DNA copy number), as well as RNA and protein expression levels for specific genes (qRT-PCR, immunoassays). Such analyses revealed a remarkable degree of molecular variation which were manifest in key phenotypic parameters such as tumor growth rate, microvasculature, and variable sensitivity to different cancer drugs.

For example, among the approximately 100 BH murine tumors, histopathologic analysis revealed subtypes each with distinct morphologic features including level of stromal cell involvement, cytokeratin staining, and cellular architecture. One subtype exhibited nested cytokeratin-positive, epithelial cells surrounded by collagen-positive, fibroblast-like stromal cells, along with slower proliferation rate, while a second subtype exhibited solid sheet, epithelioid malignant cells with little stromal involvement, and faster proliferation rates. These and other subtypes are also distinguishable by their gene expression profiles.

### Example 2

### Identification of Tivozanib PGS

Tumors in the BH murine tumor archive were tested for sensitivity to treatment with tivozanib. Evaluation of tumor response to this drug treatment was performed essentially as follows. Subcutaneously transplanted tumors were established by injecting physically disrupted tumor cells (mixed with Matrigel) into 6 week-old female BALB/c nude mice. When the tumors reached approximately 100-200 mm3, 20 tumor-bearing mice were randomized into two groups. Group 1 received vehicle. Group 2 received tivozanib at 5 mg/kg daily by oral gavage. Tumors were measured twice per week by a caliper, and tumor volume was calculated.

These studies revealed significant tumor-to-tumor variation in growth inhibition in response to tivozanib. The variation in response was expected, because the mouse model tumors had been propagated from spontaneously arising tumors, and were therefore expected to contain differing sets of secondary de novo mutations that contributed to tumorogenesis. The variation in drug response was useful and desirable, because it modeled the tumor-to-tumor variation drug response displayed by naturally occurring human tumors. Tivozanib-sensitive tumors and tivozanib-resistant tumors were identified (classified) on the basis of tumor growth inhibition, histopathology and IHC (CD31). Typically, tivozanib-sensitive tumors exhibited no tumor progression (by caliper measurement), and close to complete tumor killing, except for the peripheries, when the tumor-bearing mice were treated with 5 mg/kg tivozanib.

Messenger RNA (approx. 6 μg) from each tumor in the BH archive was amplified and hybridized, using a custom Agilent microarray (Agilent mouse 40K chip). Conventional microarray technology was used to measure the expression of approximately 40,000 genes in tissue samples from each of the 66 tumors. Comparison of the gene expression profile of a mouse tumor sample to control sample (universal mouse reference RNA from Stratagene, cat. #740100-41) was performed, and commercially available feature extraction software (Agilent Technologies, Santa Clara, Calif.) was used for feature extraction and data normalization.

Differences between tivozanib-sensitive tumors and tivozanib-resistant tumors, with respect to average (aggregate) expression of genes in different transcription clusters, were evaluated using a Student's t-test. The t-test was performed essentially as follows. Gene expression values from the microarray analysis described above were used to calculate a cluster score for each transcription cluster in each tumor. Then a p-value for each transcription cluster was calculated by applying a two-sample t-test comparing tivozanib-sensitive tumors and tivozanib-resistant tumors. False discovery rates (FDR) also were calculated. The p-values and false discovery rates for the ten highest-scoring transcription clusters are shown in Table 4.

TABLE 4
Student's t-Test Results for Transcription Cluster Expression in
Tivozanib-Sensitive Tumors and Tivozanib-Resistant Tumors

| TC No. | Structure/Function | p-value | FDR |
|---|---|---|---|
| TC50 | Myeloid cells | 4E−04 | 0.003 |
| TC48 | Hematopoietic cell; dendritic cell; | 0.001 | 0.004 |
| | monocyte enriched | | |
| TC46 | Hematopoietic cells; CD68 cell enriched | 0.003 | 0.005 |
| TC4 | Basiloid epithelial genes | 0.004 | 0.005 |
| TC5 | Epithelial phenotype, desmosomal structure | 0.004 | 0.005 |
| TC42 | | 0.004 | 0.005 |
| TC9 | | 0.009 | 0.009 |
| TC6 | | 0.012 | 0.011 |
| TC38 | | 0.015 | 0.011 |
| TC8 | | 0.017 | 0.011 |

Transcription clusters with a false discovery rate greater than 0.005 were eliminated from further consideration. Two transcription clusters, i.e., TC50 and TC48 were identified as having a false discovery rate lower than 0.005. TC50 was identified as having the lowest false discovery rate, i.e., 0.003. High expression of TC50 correlates with tivozanib resistance.

This example demonstrates the power of the disclosed method. In this example, mathematical analysis of conventional microarray expression profiling led to TC50, which is associated with certain subsets of myeloid cells that can mediate non-VEGF-dependent angiogenesis, thereby providing a mechanism of tivozanib resistance.

### Example 3

### Predicting Murine Response to Tivozanib

The predictive power of the tivozanib PGS (TC50) identified in Example 2 was evaluated in an experiment involving a population of 25 tumors previously classified as tivozanib-sensitive or tivozanib-resistant, based on actual drug response testing with tivozanib, as described in Examples 1 and 2. These 25 tumors were from a proprietary archive of primary mouse tumors in which the driving oncogene is HER2. In this example, the PGS employed was the following 10-gene subset from TC50:

MRC1

ALOX5AP

TM6SF1

CTSB

FCGR2B

TBXAS1

MS4A4A

MSR1

NCKAP1L

FLI1

A PGS score for each of the tumors was calculated from gene expression data obtained by conventional microarray analysis. We calculated the tivozanib PGS score according to the following algorithm:

$$P G S . score = \frac{1}{n} * {\sum }_{i = 1}^{n} Ei$$

wherein E1, E2, . . . En are the expression values of the n genes in the PGS.

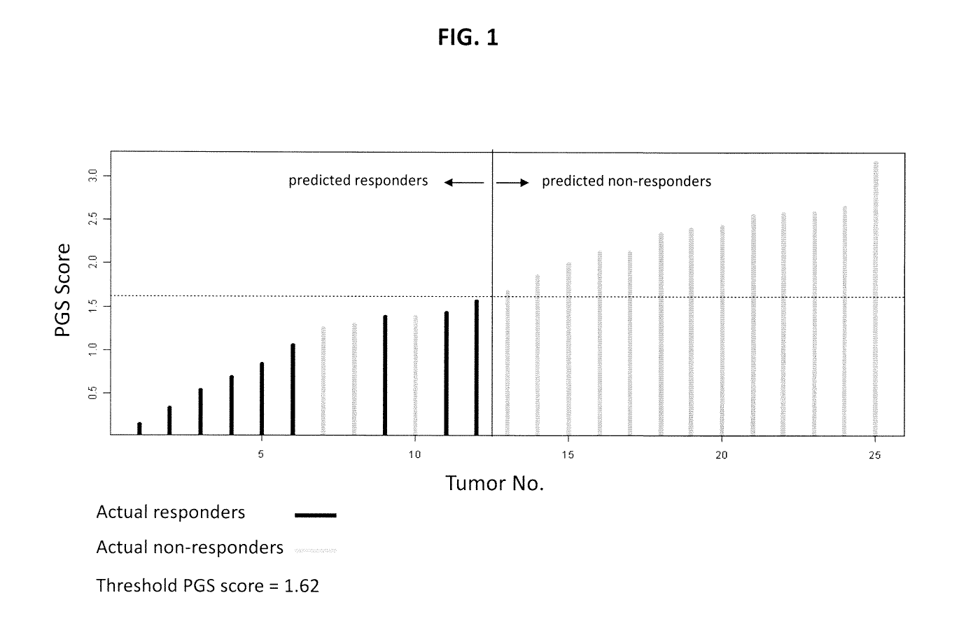

The data from this experiment are summarized as a waterfall plot shown in FIG. 1. The optimum threshold PGS score was empirically determined to be 1.62 in a threshold determination analysis, using ROC curve analysis. The results from the ROC curve analysis are summarized in FIG. 2.

When this threshold was applied, the test yielded a correct prediction of tivozanib-sensitivity (response) or tivozanib-resistance (non-response) for 22 out of the 25 tumors (FIG. 1). In predicting tivozanib resistance, the false positive rate was 25% and the false negative rate was 0%. The statistical significance of this result was assessed by applying Fisher's exact test (Fisher, 1922, J. Royal Statistical Soc. 85:87-94; Agresti, 1992, Statistical Science 7:131-153) to estimate p-value of the enrichment for responders. The contingency table for the Fisher's exact test in this case is shown in Table 5 (below):

TABLE 5
Contingency Table for Tivozanib Response Predictions

| | Actually | Actually | |
|---|---|---|---|
| | Sensitive | Resistant | Total |
| Called Sensitive | 9 | 3 | 12 |
| Called Resistant | 0 | 13 | 13 |
| Total | 9 | 16 | 25 |

In this example, the Fisher's exact test p-value was 0.00722, which is the probability of observing this test result due to chance alone. This p-value is 6.9-fold better than the conventional cut-off for statistical significance, i.e., p=0.05.

### Example 4

### Identification of Rapamycin PGS

Tumors from the BH murine tumor archive were tested for sensitivity to treatment with rapamycin (also known as sirolimus, or RAPAMUNE®). Evaluation of tumor response to rapamycin treatment was performed essentially as follows. Subcutaneously transplanted tumors were established by injecting physically disrupted tumor cells (primary tumor material), mixed with Matrigel, into 6 week-old female BALB/c nude mice. When the tumors reached approximately 100-200 mm3, 20 tumor-bearing mice were randomized into two groups. Group 1 received vehicle. Group 2 received rapamycin at 0.1 mg/kg daily, by intraperitoneal injection. Tumors were measured twice per week by a caliper, and tumor volume was calculated. These studies revealed significant tumor-to-tumor variation in growth inhibition in response to rapamycin. Rapamycin-resistant tumors were defined as those exhibiting 50% tumor growth inhibition or less. Rapamycin-sensitive tumors were defined as those exhibiting more than 50% tumor growth inhibition. Out of 66 tumors tested, 41 were found to be rapamycin-sensitive, and 25 were found to be rapamycin-resistant.

Preparation of mRNA from the tumors, and microarray analysis, were as described above in Example 2. To identify differences between rapamycin-sensitive and rapamycin-resistant tumors with respect to enrichment of expression of the 51 transcription clusters, we applied Gene Set Enrichment Analysis (GSEA) to the RNA expression data from the 41 rapamycin-sensitive tumors, and the 25 rapamycin-resistant tumors. (For a discussion of GSEA, see Subramanian et al., 2005, “Gene set enrichment analysis: A knowledge-based approach for interpreting genome-wide expression profiles,” Proc. Natl. Acad. Sci. USA 102: 15545-15550.)

Application of GSEA to the RNA expression data revealed significant differences between the rapamycin-sensitive group and the rapamycin-resistant group, with respect to expression of the 51 transcription clusters. Table 6 (below) shows GSEA results for the sensitive group of tumors. When ranked by false discovery rate q-value, the transcription cluster most enriched for high expression was found to be TC33.

TABLE 6
GSEA Results for Rapamycin-Sensitive Tumors

| TC | TC | Enrichment | Normal- | NOM | FDR | FWER |
|---|---|---|---|---|---|---|
| No. | Size | Score (ES) | ized ES | p-val | q-val | p-val |
| TC33 | 55 | 0.457 | 1.84 | 0 | 0.01228 | 0.024 |
| TC4 | 61 | 0.429 | 1.78 | 0.0020921 | 0.014881 | 0.044 |
| TC46 | 56 | 0.428 | 1.73 | 0 | 0.014995 | 0.06 |
| TC5 | 76 | 0.436 | 1.89 | 0 | 0.016654 | 0.017 |
| TC45 | 66 | 0.403 | 1.69 | 0 | 0.019452 | 0.096 |
| TC20 | 39 | 0.413 | 1.56 | 0.0081466 | 0.049047 | 0.261 |
| TC49 | 71 | 0.357 | 1.54 | 0.0201794 | 0.051305 | 0.312 |
| TC44 | 73 | 0.349 | 1.49 | 0.0064378 | 0.066288 | 0.413 |
| TC32 | 105 | 0.311 | 1.46 | 0.0200445 | 0.073882 | 0.483 |

Table 7 (below) shows GSEA results for the resistant group of tumors. When ranked by false discovery rate q-value, the transcription cluster most enriched for high expression was found to be TC26.

TABLE 7
GSEA Results for Rapamycin-Resistant Tumors

| TC | TC | Enrichment | Normal- | NOM | FDR | FWER |
|---|---|---|---|---|---|---|
| No. | Size | Score (ES) | ized ES | p-val | q-val | p-val |
| TC26 | 457 | −0.58124 | −3.16945 | 0 | 0 | 0 |
| TC29 | 136 | −0.61456 | −2.89823 | 0 | 0 | 0 |
| TC43 | 35 | −0.65415 | −2.41135 | 0 | 0 | 0 |
| TC27 | 176 | −0.44451 | −2.14628 | 0 | 2.16E−04 | 0.001 |
| TC24 | 207 | −0.4032 | −1.9709 | 0 | 0.001706 | 0.008 |
| TC25 | 36 | −0.5086 | −1.88151 | 0 | 0.004086 | 0.025 |
| TC18 | 19 | −0.5331 | −1.645 | 0.019724 | 0.027531 | 0.169 |
| TC8 | 48 | −0.37772 | −1.47427 | 0.037838 | 0.095698 | 0.536 |
| TC28 | 58 | −0.35814 | −1.45585 | 0.033808 | 0.098756 | 0.587 |
| TC17 | 32 | −0.34812 | −1.23563 | 0.182149 | 0.351789 | 0.97 |

Top enriched transcription cluster for rapamycin-sensitive tumors (TC33), and the top enriched transcription cluster for rapamycin-resistant tumors (TC26) were used to generate a 20-gene rapamycin PGS, which consists of 10 genes from TC33 and 10 genes from TC26. This particular rapamycin PGS contains the following 20 genes:

| TC33 | TC26 |
|---|---|
| FRY | DTL |
| HLF | CTPS |
| HMBS | GINS2 |
| RCAN2 | GMNN |
| HMGA1 | MCM5 |
| ITPR1 | PRIM1 |
| ENPP2 | SNRPA |
| SLC16A4 | TK1 |
| ANK2 | UCK2 |
| PIK3R1 | PCNA |

Since the PGS contains 10 genes that are up-regulated in sensitive tumors and 10 genes that are up-regulated in resistant tumors, the following algorithm was used to calculate the rapamcin PGS score:

$$P G S . score = ( \frac{1}{m} * {\sum }_{i = 1}^{m} Ei - \frac{1}{n} * {\sum }_{j = 1}^{n} Fj ) / 2$$

wherein E1, E2, . . . Em are the expression values of the m-gene signature up-regulated in sensitive tumors (TC33); and wherein F1, F2, . . . Fn are the expression values of the n-gene signature upregulated in resistant tumors (TC26). In the example above, m is 10, and n is 10.

### Example 5

### Predicting Murine Response to Rapamycin

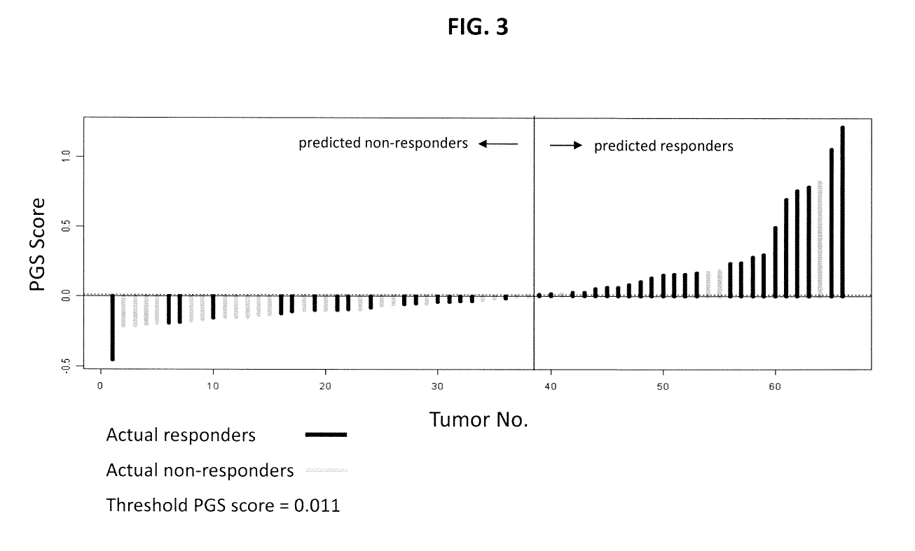

The predictive power of the rapamycin PGS identified in Example 4 was evaluated in an experiment involving a population of 66 tumors previously classified as rapamycin-sensitive or rapamycin-resistant, based on actual drug response testing with rapamycin, as described in Examples 4. These 66 tumors were from a proprietary archive of primary mouse tumors in which the driving oncogene is HER2. A rapamycin PGS score for each tumor was calculated from gene expression data obtained by conventional microarray analysis. The data from this experiment are summarized as a waterfall plot shown in FIG. 3. The optimum threshold PGS score was empirically determined to be 0.011, in a threshold determination analysis, using ROC curve analysis. The results from the ROC curve analysis are summarized in FIG. 4.

When this threshold was applied, the test yielded a correct prediction of rapamycin-sensitivity (response) or rapamycin-resistance (non-response) with regard to 45 out of the 66 tumors (FIG. 3), i.e., 68.2%. In predicting rapamycin resistance, the false positive rate was 16% and the false negative rate was 41%. The statistical significance of this result was assessed by applying Fisher's exact test (Fisher, supra; Agresti, supra) to estimate p-value of the enrichment for responders. The contingency table for the Fisher's exact test in this case is shown in Table 8.

TABLE 8
Contingency Table for Rapamycin Response Predictions

| | Actually | Actually | |
|---|---|---|---|
| | Sensitive | Resistant | Total |
| Called Sensitive | 24 | 4 | 28 |
| Called Resistant | 17 | 21 | 38 |
| Total | 41 | 25 | 66 |

In this example, the Fisher's exact test p-value was 0.000815. This means the probability of observing this test due to chance alone was 0.000815, which is the probability of observing this test result due to chance alone. This p-value is 61.4-fold better than the conventional cut-off for statistical significance, i.e., p=0.05.

### Example 6

### Identification of Breast Cancer Prognosis PGS

A population of 295 breast tumors (NKI breast cancer dataset) was used to separate tumors that have a short interval to distant metastases (poor prognosis, metastasis within 5 years) from tumors that have a long interval to distant metastases (good prognosis, no metastasis within 5 years). Among the 295 NKI breast tumors, 196 samples were good prognostic and 78 samples were bad prognostic.

Differentially expressed gene sets representing biological pathways were identified when 196 good prognosis tumors from the NKI breast dataset were compared against 78 poor prognosis tumors from the NKI breast dataset. Differences in enrichment of pathway gene lists between good prognosis and poor prognosis tumors were evaluated by employing Gene Set Enrichment Analysis (GSEA) with respect to the 51 transcription clusters. Our analysis in comparing good prognosis tumors to poor prognosis tumors demonstrated that of the transcription clusters whose member genes exhibited a significant difference in expression, TC35 (associated with ribosomes), is the top over-expressed transcription cluster in the good prognosis group (Table 9).

TABLE 9
GSEA Results for Good Prognosis Tumors

| TC | TC | Enrichment | Normal- | NOM | FDR | FWER |
|---|---|---|---|---|---|---|
| No. | Size | Score (ES) | ized ES | p-val | q-val | p-val |
| TC35 | 64 | 0.82 | 3.63 | 0 | 0 | 0 |
| TC41 | 36 | 0.66 | 2.53 | 0 | 0 | 0 |
| TC45 | 51 | 0.57 | 2.37 | 0 | 0 | 0 |
| TC40 | 56 | 0.51 | 2.18 | 0 | 0.0010633 | 0.003 |
| TC17 | 19 | 0.57 | 1.85 | 0.005848 | 0.0105018 | 0.033 |
| TC16 | 25 | 0.52 | 1.81 | 0.0059524 | 0.0108616 | 0.041 |
| TC44 | 52 | 0.42 | 1.74 | 0.0039841 | 0.0162979 | 0.072 |
| TC22 | 24 | 0.47 | 1.64 | 0.0143678 | 0.0310619 | 0.15 |
| TC46 | 45 | 0.39 | 1.61 | 0.0067568 | 0.0330688 | 0.179 |
| TC42 | 25 | 0.46 | 1.58 | 0.042623 | 0.0344636 | 0.205 |

TC26 (associated with proliferation) is the top over-expressed cluster in the poor prognosis group, as shown in the GSEA results presented in Table 10.

TABLE 10
GSEA Results for Poor Prognosis Tumors

| TC | TC | Enrichment | Normal- | NOM | FDR | FWER |
|---|---|---|---|---|---|---|
| No. | Size | Score (ES) | ized ES | p-val | q-val | p-val |
| TC26 | 301 | −0.62945 | −2.85486 | 0 | 0 | 0 |
| TC27 | 111 | −0.61451 | −2.50536 | 0 | 0 | 0 |
| TC30 | 37 | −0.62567 | −2.08285 | 0 | 0 | 0 |
| TC34 | 33 | −0.62657 | −2.07428 | 0 | 0 | 0 |
| TC43 | 25 | −0.6238 | −1.91291 | 0 | 9.62E−04 | 0.006 |
| TC49 | 62 | −0.4897 | −1.82795 | 0 | 0.003755 | 0.028 |
| TC32 | 76 | −0.47135 | −1.81733 | 0 | 0.003933 | 0.034 |

The most enriched transcription cluster for the good prognosis tumors (TC35), and the most enriched transcription cluster for the poor prognosis tumors (TC26) were used to generate a 20-gene breast cancer prognosis PGS, which consists of ten genes from TC35 and ten genes from TC26. This particular breast cancer PGS contains the following 20 genes:

| TC35 | TC26 |
|---|---|
| RPL29 | DTL |
| RPL36A | CTPS |
| RPS8 | GINS2 |
| RPS9 | GMNN |
| EEF1B2 | MCM5 |
| RPS10P5 | PRIM1 |
| RPL13A | SNRPA |
| RPL36 | TK1 |
| RPL18 | UCK2 |
| RPL14 | PCNA |

Since the breast cancer prognosis PGS contains 10 genes that are up-regulated in good prognosis tumors and 10 genes that are up-regulated in poor prognosis tumors, the following algorithm was used to calculate the breast cancer prognosis PGS scores:

$$P G S . score = ( \frac{1}{m ,} * {\sum }_{i = 1}^{m} Ei - \frac{1}{n} * {\sum }_{j = 1}^{n} Fj ) / 2$$

wherein E1, E2, . . . Em are the expression values of the m-gene signature up-regulated in good prognosis tumors (TC35); and wherein F1, F2, . . . Fn are the expression values of the n-gene signature upregulated in poor prognosis tumors (TC26). In the example above, m is 10, and n is 10.

### Example 7

### Validation of Breast Cancer Prognosis PGS

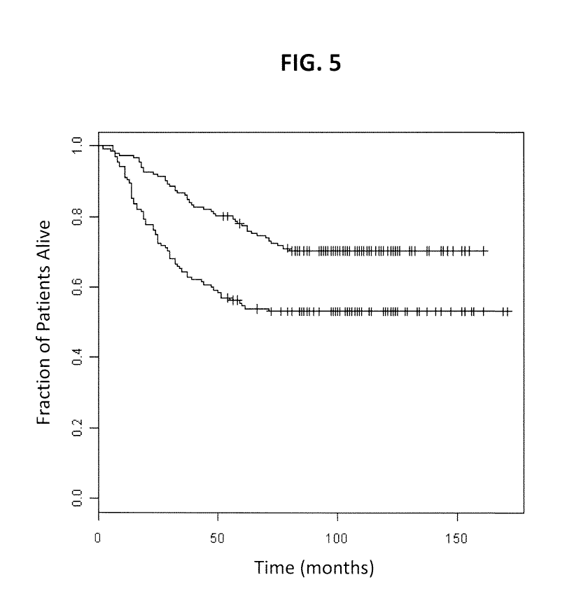

The prognostic PGS identified in Example 6 (above) was validated in an independent breast cancer dataset, i.e., the Wang breast cancer dataset (Wang et al., 2005, Lancet 365:671-679). A population of 286 breast tumors from the Wang breast cancer dataset was used as an independent validation dataset. The samples in Wang datasets had clinical annotation including Overall Survival Time and Event (dead or not). The 20-gene breast cancer prognostic PGS identified in Example 6 was an effective predictor of patient outcome. This is shown in FIG. 5, which is a comparison of Kaplan-Meier survivor curves. This Kaplan-Meier plot shows the percentage of patients surviving versus time (in months). The upper curve represents patients with high PGS scores (scores above the threshold), which patients achieved relatively longer actual survival. The lower curve, represents patients with low PGS scores (scores below the threshold), which patients achieved relatively shorter actual survival. Cox proportional hazards regression model analysis showed that the PGS generated from TC35 and TC26 is an effective prognostic biomarker, with a p-value of 4.5e-4, and a hazard ratio of 0.505.

### Example 8

### Predicting Human Response

The following prophetic example illustrates in detail how the skilled person could use the disclosed methods to predict human response to tivozanib, using TaqMan® data.

With regard to a given tumor type (e.g., renal cell carcinoma), tumor samples (archival FFPE blocks, fresh samples or frozen samples) are obtained from human patients (indirectly through a hospital or clinical laboratory) prior to treatment of the patients with tivozanib. Fresh or frozen tumor samples are placed in 10% neutral-buffered formalin for 5-10 hours before being alcohol dehydrated and embedded in paraffin, according to standard histology procedures.

RNA is extracted from 10 μm FFPE sections. Paraffin is removed by xylene extraction followed by ethanol washing. RNA is isolated using a commercial RNA preparation kit. RNA is quantitated using a suitable commercial kit, e.g., the RiboGreen® fluorescence method (Molecular Probes, Eugene, Oreg.). RNA size is analyzed by conventional methods.

Reverse transcription is carried out using the SuperScript™ First-Strand Synthesis Kit for qRT-PCR (Invitrogen). Total RNA and pooled gene-specific primers are present at 10-50 ng/μl and 100 nM (each), respectively.

For each gene in the PGS, qRT-PCR primers are designed using commercial software, e.g., Primer Express® software (Applied Biosystems, Foster City, Calif.). The oligonucleotide primers are synthesized using a commercial synthesizer instrument and appropriate reagents, as recommended by the instrument manufacturer or vendor. Probes are labeled using a suitable commercial labeling kit.

TaqMan® reactions are performed in 384-well plates, using an Applied Biosystems 7900HT instrument according to the manufacturer's instructions. Expression of each gene in the PGS is measured in duplicate 5 μl reactions, using cDNA synthesized from 1 ng of total RNA per reaction well. Final primer and probe concentrations are 0.9 μM (each primer) and 0.2 μM, respectively. PCR cycling is carried out according to a standard operating procedure. To verify that the qRT-PCR signal is due to RNA rather than contaminating DNA, for each gene tested, a no RT control is run in parallel. The threshold cycle for a given amplification curve during qRT-PCR occurs at the point the fluorescent signal from probe cleavage grows beyond a specified fluorescence threshold setting. Test samples with greater initial template exceed the threshold value at earlier amplification cycles.

To compare gene expression levels across all the samples, normalization based on five reference genes (housekeeping genes whose expression level is similar across all samples of the evaluated tumor type) is used to correct for differences arising from variation in RNA quality, and total quantity of RNA, in each assay well. A reference C_{T} (threshold cycle) for each sample is defined as the average measured C_{T} of the reference genes. Normalized mRNA levels of test genes are defined as ΔC_{T}, where ΔC_{T} reference gene C_{T} minus test gene C_{T}.

The PGS score for each tumor sample is calculated from the gene expression levels, according to the algorithm set forth above. The actual response data associated with tested tumor samples are obtained from the hospital or clinical laboratory supplying the tumor samples. Clinical response is typically defined in terms of tumor shrinkage, e.g., 30% shrinkage, as determined by suitable imaging technique, e.g., CT scan. In some cases, human clinical response is defined in terms of time, e.g., progression free survival time. The optimal threshold PGS score for the given tumor type is calculated, as described above. Subsequently, this optimal threshold PGS score is used to predict whether newly-tested human tumors of the same tumor type will be responsive or non-responsive to treatment with tivozanib.

### INCORPORATION BY REFERENCE

The entire disclosure of each of the patent documents and scientific articles cited herein is incorporated by reference for all purposes.

### EQUIVALENTS

The invention can be embodied in other specific forms with departing from the essential characteristics thereof. The foregoing embodiments therefore are to be considered illustrative rather than limiting on the invention described herein. The scope of the invention is indicated by the appended claims rather than by the foregoing description, and all changes that come within the meaning and range of equivalency of the claims are intended to be embraced therein.

We claim:

1. A method of identifying a human tumor as likely to be sensitive or resistant to treatment with tivozanib, the method comprising:
(i) measuring, in a sample from the tumor, the relative expression level of each gene in a predictive gene set (PGS), wherein the PGS comprises at least 10 of the genes from TC50; and
(ii) calculating a PGS score according to the algorithm
$$P G S . score = \frac{1}{n} * {\sum }_{i = 1}^{n} Ei$$
wherein E1, E2, . . . En are the expression values of the n genes in the PGS, and
wherein a PGS score below a defined threshold indicates that the tumor is likely to be sensitive to tivozanib, and a PGS score above the defined threshold indicates that the tumor is likely to be resistant to tivozanib.

2. The method of claim 1, wherein the PGS comprises a 10-gene subset of TC50 selected from the group consisting of:
(a) MRC1, ALOX5AP, TM6SF1, CTSB, FCGR2B, TBXAS1, MS4A4A, MSR1, NCKAP1L, and FLI1; and
(b) LAPTM5, FCER1G, CD48, BIN2, C1QB, NCF2, CD14, TLR2, CCL5, and CD163.

3. The method of claim 1, further comprising the step of performing a threshold determination analysis, thereby generating a defined threshold, wherein the threshold determination analysis comprises a receiver operator characteristic curve analysis.

4. The method of claim 1, wherein the relative expression level of each gene in the PGS is measured by a method selected from the group consisting of: (a) DNA microarray analysis, (b) qRT-PCR analysis, (c) qNPA analysis, (d) a molecular barcode-based assay, and (e) a multiplex bead-based assay.
